(19) United States
(12) Patent Application Publication
(10) Pub. No.: US 2022/0008141 A1
(43) Pub. Date: Jan. 13, 2022

(54) ENHANCED REALITY MEDICAL GUIDANCE SYSTEMS AND METHODS OF USE

(71) Applicant: Petal Surgical, Inc., Foster City, CA (US)

(72) Inventors: Prashant CHOPRA, Foster City, CA (US); Salil S. JOSHI, Germantown, TN (US)

(21) Appl. No.: 17/448,859

(22) Filed: Sep. 24, 2021

Related U.S. Application Data
(63) Continuation of application No. 16/336,388, filed on Mar. 25, 2019.
Application No. 16/336,388 is a continuation-in-part of application No. 15/493,075, filed on Apr. 20, 2017, now abandoned.
(60) Provisional application No. 62/404,002, filed on Oct. 4, 2016.
(60) Provisional application No. 62/404,002, filed on Oct. 4, 2016.

(51) Int. Cl. A61B 34/20; A61B 90/00; G06F 1/16; A61B 5/00; A61B 6/00; A61B 8/00; G06F 3/01

(52) U.S. Cl. CPC A61B 34/20; A61B 90/39; G06F 1/163; A61B 90/36; A61B 90/37; A61B 5/489; A61B 2017/00221; A61B 6/5247; A61B 8/4245; G06F 3/011; A61B 2034/2048; A61B 2090/365; A61B 5/742

(57) ABSTRACT

Apparatus, system and methods are described for providing a health care provider (HCP) with an enhanced reality perceptual experience for surgical, interventional, therapeutic, and diagnostic use. The apparatus, system and methods make use of a combination of sensors and audio visual data to cross-correlate information, and present the correlated information to the HCP on to one or more platforms for use during a diagnostic, interventional, therapeutic, or surgical procedure.

### CROSS REFERENCE TO RELATED APPLICATIONS

This application is a continuation of U.S. patent application Ser. No. 16/336,388, filed on May 22, 2019, titled “ENHANCED REALITY MEDICAL GUIDANCE SYSTEMS AND METHODS OF USE,” which is a U.S. National Phase Application Under 35 U.S.C. § 371 of International Application No. PCT/US17/54868, filed on Oct. 3, 2017, titled “ENHANCED REALITY MEDICAL GUIDANCE SYSTEMS AND METHODS OF USE,” now PCT Publication No. WO 2018/067515, which claims the benefit of U.S. Provisional Patent Application No. 62/404,002, filed on Oct. 4, 2016, titled “ENHANCED REALITY MEDICAL GUIDANCE SYSTEMS AND METHODS OF USE,” and is a continuation-in-part of U.S. patent application Ser. No. 15/493,075, filed on Apr. 20, 2017, titled “ENHANCED REALITY MEDICAL GUIDANCE SYSTEMS AND METHODS OF USE,” now abandoned, which claims the benefit of U.S. Provisional Patent Application No. 62/404,002, filed on Oct. 4, 2016, each of which is herein incorporated by reference in its entirety.

### INCORPORATION BY REFERENCE

All publications and patent applications mentioned in this specification are incorporated herein by reference in their entirety to the same extent as if each individual publication or patent application was specifically and individually indicated to be incorporated by reference.

### BACKGROUND

Augmented reality (AR) technology is finding more and more widespread use for entertainment and industrial applications. Healthcare applications are also starting to see a rise in the interest in use of AR technologies to improve medical procedures, clinical outcomes, and long term patient care. Augmented reality technologies may also be useful for enhancing the real environments in the patient care setting with content specific information to improve patient outcomes. However, due to certain fundamental challenges that limit the accuracy and usability of AR in life critical situations, the use of AR is yet to realize its complete potential in healthcare space. AR can generally be thought of as computer images overlaid on top of real images with the computer-generated overlay images being clearly and easily distinguishable from the real-world image. An example of AR use is the video game Pokémon Go™ which has an AR mode when players try to catch Pokémon virtually placed in the real world, anchored to real geographical co-ordinates or features. Virtual Reality (VR) can generally be thought of as a fully computer simulated environment where the user does not view anything from the real world, but only sees the virtual environment created by a computer. VR requires the use of goggles or headsets that prohibit a user from seeing the real world while the user is in the virtual reality.

### SUMMARY OF THE DISCLOSURE

Described herein are various devices, systems and methods for combining various kinds of medical data to produce a new visual reality for a surgeon or health care provider. The new visual reality provides a user with the normal vision of the user's immediate surroundings accurately combined with a virtual three-dimensional model of the operative space and tools, enabling a user to ‘see’ through the opaque parts of a patient body, and into the patient to see a virtual representation of the operative space and clinical tools, without cutting open the patient.

In some embodiments, there is a method of producing visual image data set from a visual image sensor containing at least one visual marker. The method comprises identifying one or more fiducial marker(s) in at least one two-dimensional image, determining a depth and an orientation of the fiducial marker from the point of view of at least one visual sensor taking an image, establishing a three dimensional (3D) coordinate system for the visual marker(s) using at least one two-dimensional image, and creating a three-dimensional image data set.

In some embodiments, there is a method of producing visual image data set from a sensor image. The method comprises establishing a three dimensional coordinate system for a three dimensional volume that is sensed by a position and an orientation sensor, sensing a position and/or an orientation of at least one of a sensor detectable device within the three dimensional volume, assigning the sensor detectable device a volume, and an orientation in the three dimensional volume and creating one or more visual image data set indicating the position, orientation and volume of the sensor detectable device in the three dimensional volume.

In some embodiments, there is a method of combining data types to create a three-dimensional image for a medical procedure. The method comprises receiving at least one data set from a medical image scanner, receiving at least one data set from a position and orientation sensor, receiving at least one data set from a visual information sensor and integrating the data sets from the medical image scan, the data set from the position and orientation sensor and the visual information sensor into a combined image.

In some embodiments, there is a fiducial marker for use in a medical procedure. The fiducial marker comprises a body, visually detectable feature visible on the surface of the body, the visually detectable feature having at least one visually distinct edge, and a plurality of sensor detectable devices, the sensor detectable devices positioned in the body wherein at least one sensor detectable device is lined up with one visually distinct edge of the visually detectable feature.

In some embodiments, at least one sensor detectable device is lined up with one visually distinct edge of the visually detectable feature. In some embodiments, the orientation and position of at least one sensor detectable device (SDD) is known relative to at least one visually detectable feature. In some embodiments, there is a wearable display device comprising a semi-transparent electronic display layer for receiving a combined image; and a structure support layer attached to the semi-transparent electronic display layer. The structure support layer may provide vision correction to a user while the semi-transparent electronic display layer provides a computer-generated image of at least one internal detail of the object the user is looking at.

In some embodiments, there is a flexible display for placement on a patient body, the flexible display comprises a flexible body able to be draped onto a patient body, the flexible body having an upper surface and a lower surface, a display screen incorporated into the upper surface, and display electronics incorporated into the flexible body. In some embodiments, a position and orientation sensor detector may be integrated with the flexible display.

In some embodiments, there is a wearable projection apparatus comprising a body having a body conforming contour, a projector incorporated into the body, the projector able to project an image onto a surface, and a position sensor able to discriminate between an acceptable image display area and a non-image display area.

In some embodiments there is a multicomponent system for producing an enhanced reality image for overlaying virtual images constructed from at least two different image sources. The system has one or more fiducial markers placed on a patient body, a wearable display device and a computer system in electronic communication with the wearable display device and a visual imaging system, the computer being capable of correlating the data from at least two different image sources, and integrating them into an enhanced reality image for display on the wearable display device.

In some embodiments there is a system for producing an enhanced reality image for overlaying virtual images constructed from at least two different image sources. The system has a sensor garment with at least one imaging device, a first detector device for recording image information of the imaging device and a computer able to correlate data from the imaging device and the first detector and match the correlated data with a scan data set. The system also has a display device.

Described herein are various devices, systems and methods for creating an enhanced reality (ER) image for use in patient treatment. Several devices are used in combination to produce an enhanced reality image. The enhanced reality image is distinguished from a virtual reality (VR) or an augmented reality (AR) in that the user of the system will still be fully present in the real world, with the ability to see their local environment through their own eyes, unassisted by any external audio/video technology. It is also distinguished from an augmented or a mixed reality in that the information presented enhances the user's perception of reality in depth, texture, focus, and/or other contextual information to assist in a critical task at hand. The enhanced reality system has a control unit, one or more sensor platforms, and a wearable display. The system may additionally include a sensor garment, a display (either a tablet or computer screen or glasses) and/or a variety of sensor platforms. The sensor platforms may be tools, guidewires, catheters or other minimally invasive tools used singly, or in combinations. The control unit may be a single computer located physically where the health care provider is (possibly also as a wearable or portable computer), or it may be a computer in a remote location. The computer may be in the cloud for wireless interaction with the system, or it may be linked by hard wire. The control unit can access medical records for a patient, similar to how doctors in medical organizations retrieve patient data in other electronically linked systems and databases.

Medical procedures may be visually intensive. Doctors and other health care providers generally need to see what they are doing in order to achieve a clinically desirable outcome. Doctors may see directly (line of sight into or onto the patient body) or indirectly using a scope. Indirect observation may include image translation of imaging tools like X-ray, Ultrasound, NMR scans, just to name a few. Direct visualization can be achieved through open surgery, or a direct imaging device inserted in the body. The systems, tools and methods described herein can provide an enhanced reality medical guidance system, that can enable an enhanced perception of medical reality and may make certain kinds of medical procedures easier for health care providers to perform without the need for expensive, large footprint, and sometimes harmful (needing radiation and contrast) imaging or diagnostic systems. The system collects one or more of image data, position data and dimensional data from various sources, and combines the image/position/dimensional (IPD) data to form the enhanced reality image. In a simplified and non-limiting example, the system can correlate IPD data from the interior of a patient, with an image from the exterior surface of the patient, and real time information about the interior of the patient. This process can be repeated using multiple sensors and views, and then the multiple views are combined and formed into a three dimensional image of the patient's internal anatomy. This combined enhanced image may also display correctly positioned tools or objects that would otherwise not be visible to the HCP unless the patient goes through harmful radiation based imaging, or invasive surgery. The image presented to the user may be depth, focus, lighting, and texture corrected (to show the enhancements out of focus when needed to match the user's point of focus and the visual context around it) and/or stereoscopic if the display allows it. The three-dimensional image can be projected into one or more video display devices, allowing the health care provider to navigate the enhanced reality image with confidence, knowing where the surgical instruments are and where the boundaries of the patient organs are. The image may build in movement like breathing, heart beats, and other bodily functions so the health care provider can see those movements accurately represented in the enhanced reality image. In this way, minimally invasive medical procedures, and other indirect procedures may be accurately visualized.

Current systems use fluoroscopy (a kind of x-ray device) to see into the patient during minimally invasive interventions. Fluoroscopy inherently is a projection based modality which combines multiple layers of varying and changing soft and hard structures into a single image. This leaves a lot of visual inference and uncertainty about the imaged structure to the observer, making procedural decisions hard during an intervention. Furthermore, fluoroscopy is not a precise soft tissue diagnostic modality since it is difficult to see soft tissue on x-ray images. Fluoroscopy is thus very frequently used with chemical markers that highlight internal soft structures, increasing the amount of radiation exposure to the patient and the clinical staff, and in many cases causing contrast induced organ malfunctions (nephropathy or kidney failure is an example for patients suffering from cardiovascular conditions typically have compromised kidney function anyways), skin burns (when used for extended periods in Cath Lab procedures), in turn leading to a reduced quality of life and increased cost of care for adverse secondary conditions, and in certain cases: an eventual loss of life.

In a non-limiting example analogy, using an enhanced reality guidance system may be thought of as like acquiring a supernatural power to see through otherwise opaque objects in a natural, safe, and accurate way to enable the user to accomplish complicated tasks (like clinical procedures) without relying on remote visual technology, or imprecise visual tools.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A shows an example of a system with various components according to an embodiment.

FIG. 1B illustrates a User Input Device (UID) and wireless interface according to an embodiment.

FIG. 1C illustrates data sources for integration according to an embodiment.

FIG. 1D illustrates individual elements in the procedural suite according to an embodiment.

FIG. 2A-2N illustrates various fiducial markers according to several embodiments.

FIGS. 3A-3H illustrate various sensor garments according to several embodiments.

FIG. 4 illustrates an energy emission seed and sensor according to an embodiment.

FIG. 5A illustrates an enhanced reality wearable display according to an embodiment.

FIG. 5B illustrates the lens elements of a wearable display according to an embodiment.

FIGS. 5C-5D show alternate image displays according to several embodiments.

FIG. 6A illustrates a cornea wearable display according to an embodiment.

FIG. 6B through 6G show some details of various displays according to several embodiments.

FIG. 7 illustrates a projector for presenting enhanced reality images onto a cornea according to an embodiment.

FIG. 8 shows a flow chart for extraction of anatomical information and integrating it with a patient data according to an embodiment.

FIG. 9 illustrates a flow chart for mixing images from various sources according to an embodiment and displaying them.

FIG. 10 illustrates a flow chart for morphing the pre-operative patient images by using live patient sensor data according to an embodiment.

FIGS. 11A-B provides an example of a patient visiting a health care provider (HCP) according to an embodiment.

FIG. 12A illustrates an example of a patient examination according to an embodiment.

FIG. 12B illustrates a pre-intervention examination according to an embodiment.

FIG. 13 provides a flow chart showing an example of data gathering for an interventional procedure according to an embodiment.

FIG. 14 provides a flow chart for an alternative embodiment of a interventional procedure according to an embodiment.

FIG. 15 provides another sample method to generate an enhanced reality image set and send it to a wearable display according to an embodiment.

FIG. 16 illustrates a process for producing an enhanced reality image according to an embodiment.

FIG. 17 illustrates a method of marker detection according to an embodiment.

FIG. 18 illustrates a method of deformable model extraction according to an embodiment.

FIG. 19 illustrates a method of pre-operative correlation of markers according to an embodiment.

FIG. 20A illustrates a method of electromagnetic position and orientation sensor data and scan image data registration according to an embodiment.

FIG. 20B illustrates an example of a system using electromagnetic position and orientation sensor data and scan image data registration according to an embodiment.

FIGS. 21A-B illustrate a method and match score display according to an embodiment.

FIGS. 22A-C illustrate a method and system for generating and displaying an enhanced reality image according to an embodiment.

FIGS. 23A-B illustrate a method of tool tracking for an enhanced reality image according to an embodiment.

FIG. 24 illustrate a method of displaying an enhanced reality image according to an embodiment.

FIGS. 25A-D illustrate devices for displaying an enhanced reality image according to several embodiments.

FIG. 26A illustrates a method of determining the position and orientation of a marker patch in a wearable's space according to an embodiment.

FIG. 26B-C illustrates an enhanced reality tool with a sensor according to an embodiment.

FIG. 27 illustrates an enhanced reality tool approaching a treatment site in a body lumen according to an embodiment.

FIGS. 28 and 29 illustrate a minimally invasive device for crossing a body lumen occlusion according to an embodiment.

FIG. 30 illustrates a steerable tool according to an embodiment.

FIG. 31 illustrates a variety of steerable guiding tubes according to several embodiments.

FIGS. 32 and 33 illustrate several guidewire locking mechanisms according to several embodiments.

FIG. 34 illustrates a guidewire having fiducial markers according to an embodiment.

FIG. 35 illustrates a use situation of the enhanced reality system according to an embodiment.

FIG. 36 illustrates a benchtop image of the current device and methods according to an embodiment.

FIG. 37 illustrates an animal image of an internal anatomy display of the systems and methods according to an embodiment.

FIG. 38A illustrates a sensor garment with a display device according to an embodiment.

FIG. 38B provides a possible use scenario for the sensor garment of FIG. 38A according to an embodiment.

FIG. 39 illustrates a glove or hand attachment for use with the enhanced reality system according to an embodiment.

FIG. 40 provides a cut-away view of a sensor garment according to an embodiment.

FIGS. 41A-B illustrate an assembly and assembled version of the sensor garment according to an embodiment.

FIGS. 42A-B illustrate another sensor garment according to an embodiment.

FIG. 43 illustrates methods of performing a safety check according to an embodiment.

FIG. 44 illustrates methods of performing a safety check according to an embodiment.

FIG. 45 illustrates an attachable viewer and camera to a pair of glasses according to an embodiment.

FIGS. 46A-B provides an illustration of a removable chamber style sensor dome according to an embodiment.

FIG. 47 illustrates a hand held device for performing live imaging for use with an enhanced reality system according to an embodiment.

### DETAILED DESCRIPTION

In the following detailed description, reference is made to the accompanying drawings, which form a part thereof. In the drawings, similar symbols typically identify similar components, unless context dictates otherwise. The illustrative embodiments described below, along with the drawings, description and claims are not meant to be limiting. Other embodiments may be utilized, and other changes may be made without departing from the spirit or scope of the subject matter presented here.

Referring to the figures generally, various embodiments disclosed herein relate to providing devices, systems and methods for improving the treatment of patients in the hands of health care providers. Some embodiments described herein relate to improving the coordination of patient data. Some embodiments described herein relate to providing an enhanced sensory environment for a health care provider when treating or working with a patient. Some embodiments described herein relate to providing care givers with near real time treatment options from analyzed data. Other embodiments described herein relate to enhanced visualization techniques combining two or more imaging and sensing technologies and presenting a combination in a way that may enhance the contextual reality. Still other embodiments relate to an interactive guidance procedure utilizing patient and procedure data, combined with treatment tools. These and other embodiments are detailed herein.

In discussing the various embodiments and drawings, several references may assist the reader in understanding the description. Generally herein, reference to a medical device may include a distal and proximal end. The distal end refers to the end that is farther away from the user or health care provider (HCP). For a minimally invasive device, the distal end generally is inserted into the patient body, while the proximal end is held by the user. Additionally, references are made herein to the “wearable” view. Several components, devices and systems described herein have a wearable device. Some are wearable by a user or HCP or the supporting clinical staff, and others are wearable by a patient before, during, or after a medical intervention. The wearable view may be context driven, as there are wearable elements for the user and the patient.

References to a display device include any device capable of rendering an image (such as a computer monitor, light engine, holographic assembly, or an optical implant in or around the human eyes) or a device that can receive a projected image (like a ‘silver’ screen).

In discussing the various embodiments herein, some notation is used to facilitate the understanding of the disclosure. The following legend is provided for some of these abbreviations and notations:

TABLE 1

| Letter | General Usage |
|---|---|
| I | Image or image data |
| MR | Magnetic Resonance Image |
| CTA | Contrast Enhanced Computed Tomography images |
| i | Denotes a ‘sample’ in space, time, or another |
| | dimension |
| Dᵢ | Data instance, ith sample |
| P | Patient |
| W | Wearable Display device |
| T | Tracker (electromagnetic or another similar position |
| | and orientation sensor equipped device) |
| E | Enhanced Reality |
| Pose | Position and Orientation, together |
| ERHM | Enhanced Reality Holographic Medium, a holographic |
| | display that floats in between the user and the object |
| | being enhanced. |

TABLE 2

| Example usage | Example meaning |
|---|---|
| Iᵢ^{CTA} | CTA scan image set, the ith sample in time. |
| Pᵢ^{T} | Patient sensor marker data in sensor world, ith |
| | sample. |
| Dᵢ^{CTA} | Data from archives in CTA space, ith sample. |
| Pᵢ^{W} | Patient visual sensor marker (fiducial markers) data in |
| | wearable's world |
| Wᵢ^{POSE} | Pose (orientation and position) of wearable display in |
| | global space, ith sample |
| Vᵢ^{W} | Virtual image, ith sample, in wearable's world from |
| | wearable point of view |
| Eᵢ^{W} | Enhanced image, ith sample, in wearable's space |
| | from wearable's point of view. |
| Iᵢ^{W} | Camera image, ith sample from Wearable's |
| | camera(s), from wearable point of view. |
| M′_{s-new} | New Transformed Marker Sensor co-ordinates, |
| | intermediate only, during optimization. |
| ₛT^{CT}_{new} | New Sensor space to CT space transform, |
| | intermediate only, during optimization |
| ₛS^{CT}_{new} | New Correlation Score between a Marker's Sensor |
| | space co-ordinates and CT space co-ordinates, |
| | intermediate only, during optimization |
| M″ₛ | Final transformed Marker Sensor co-ordinates in CT |
| | space |
| M′_{I} | Enhanced Reality Marker co-ordinates in wearable |
| | camera's image (I) space |
| _{I}S^{CT} | Correlation Score between a Marker's Camera Image |
| | space co-ordinates and CT space co-ordinates |
| I_{c} | Wearable camera's image |
| T_{CT} | Tool Sensor co-ordinates in CT space |
| D_{md} | Depth of model from wearable display or camera (in |
| | a tablet's case they are in the same plane) |
| d_{f} | Sensed depth of User's focus, where the eyes are |
| | focused, and left and right lines of sight intersect. |
| Pᵢ^{T} | Marker data in Patient space, ith instance |
| Dᵢ^{T} | Marker data in pre-operative Data space, ith instance |
| Mᵢ^{W} | Mixed reality images in Wearable Space, ith instance. |
| Iᵢ^{W} | Wearable camera image, ith instance |
| Dₘ | Depth of Marker in camera space |

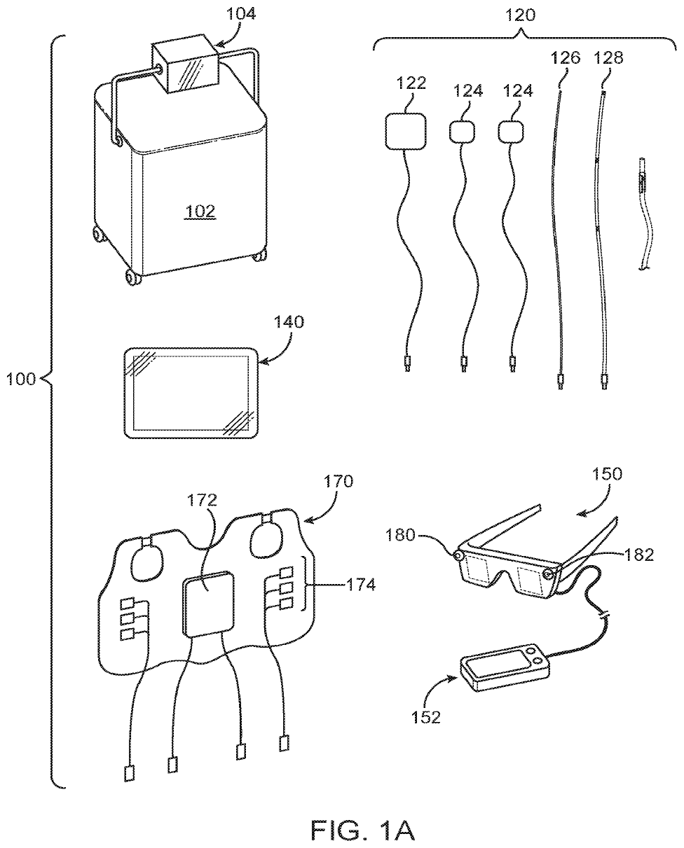

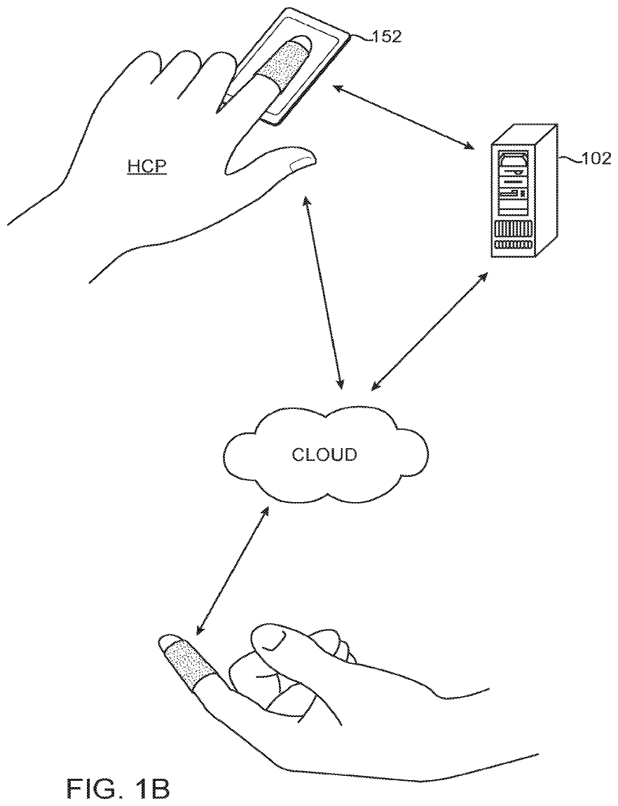

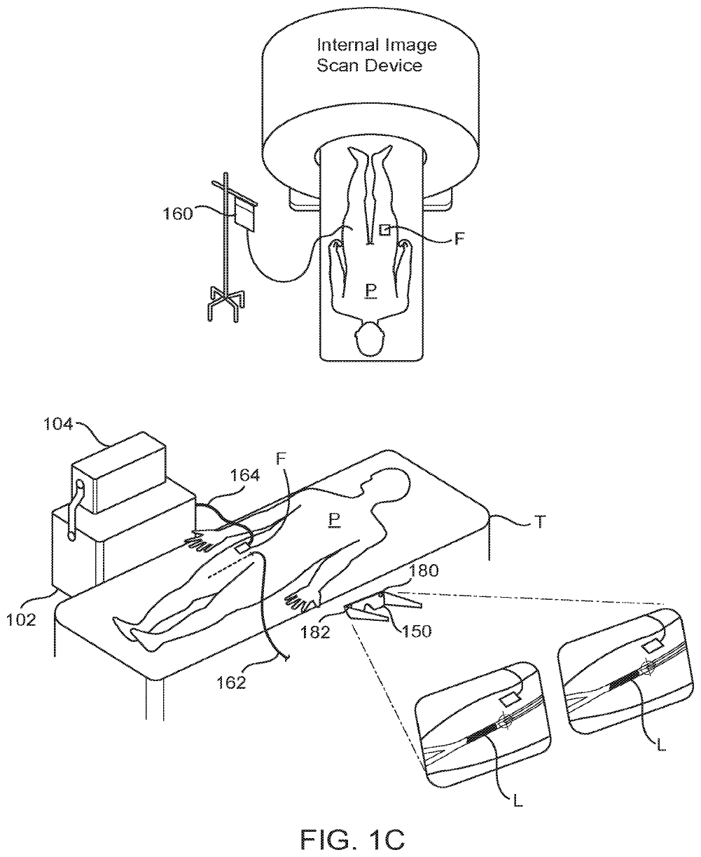

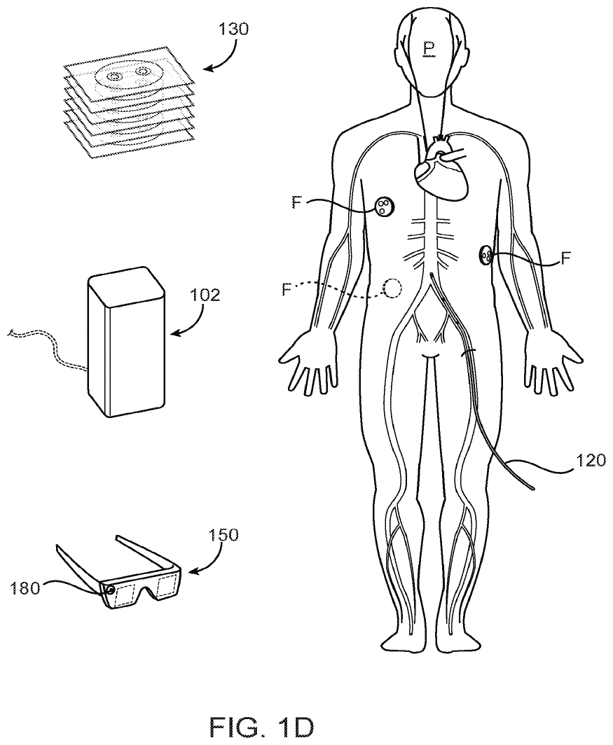

In an embodiment, there may be a visualization system for enhancing localized view of a body space. The system 100 may have a control unit 102 with an electromagnetic field sensor 104 (FIG. 1A). The electromagnetic field sensor may be a point of origin or reference for a 3D/4D coordinate system within the health care provider (HCP) service room or interventional suite. A variety of sensing devices 120 may be used with the system in any combination. In some embodiments, there may be one or more of: a large electromagnetic patient sensor 122, a small electromagnetic patient sensor 124, a guidewire 126 having a built-in sensor, and/or some other form of minimally invasive device with a sensor 128. In some embodiments, the sensor element may be a detector element. In still other embodiments, the devices with sensors may also have detectors. In various embodiments, the term “probe” can mean a probe with sensors, energy emitters, detectors, radiopaque markers or other elements that can be detected by a sensor, or detect data or energy emissions, can perform a scanning operation (e.g. ultrasound imaging, micro x-ray detection, micro x-ray emission, or other modalities) and export detected signals to a control unit. The system may have an optional tablet 140 or computer screen for viewing information, video, pictures and/or computer generated images. In some embodiments, the system may use enhanced reality goggles 150 in conjunction with, or in place of, the tablet or computer screen 140. In some embodiments there may be a screen incorporated to a sensor garment placed on the patient. In some embodiments, a user may opt for imaging devices in more than one location (goggles, display screen, on patient, on physician's hands or body, remote display, or any surface in the natural field of view of the physician etcetera.) A user input device (UID) 152 may be used with the system so the user can enter commands into the system and control some or all the operating features of the visualization system. The UID 152 may be a wired or wireless device held in one hand, or a larger device presented in a usable work space in reach of the HCP. In one aspect, the UID may be a wearable device connected to the goggles, so the user may engage the UID to change the view or options presented on the goggles or computer screen. In another aspect, the UID 152 may be incorporated into the goggles 150 so the user may interact with the goggles to change views or options of the audio/visual information presented in the goggles or on computer screen 140. The goggles may have a wireless or wired interface to get audio signal to the HCP wearing the goggles. The goggles 150 may use wireless signals to communicate data to the control unit 102. In some embodiments, the goggles 150 may communicate to the control unit via a hard wire. In some embodiments, the goggles may also have a tracking unit or other device so that the goggles may be tracked in space relative to the patient, the control unit or some other defined point of origin. In some aspects, the position of the goggles can be accurately measured relative to the origin. The various sensor units may have a data connection to the control unit that is wireless, or hard wired. In embodiments where they are wirelessly connected, the sensor units may operate on internal power (i.e. a battery). In embodiments where the sensor elements are physically connected to the control unit, the sensor elements can draw power from the control unit. In some embodiments, there may be an intermediate unit between the control unit and the sensor elements. The intermediate unit may provide power and data relay between the control unit and the senor units. In embodiments where the sensor elements are physically connected to the control unit, or intermediate unit, the sensor elements may plug in via any established connection type (e.g. universal serial bus (USB), small computer system interface (SCSI), parallel connection, Thunderbolt™, high-definition multimedia interface (HDMI) or other connections yet to be created) or a novel connection type established in particular for the intended use. In some embodiments the user may opt to see any image data available used to construct the enhanced reality image.

In some embodiments, a wearable sensor garment 170 may be used. The sensor garment 170 may take many forms. It could be a vest for use on the chest, or a wrap-around sleeve that may be fitted to a patient's arm or leg. The garment 170 might be fitted to a hat or helmet for use on the head, or adapted to fit over or around any part of the body. The wearable sensor garment may be designed as loose fitted clothing to fit over a patient's anatomy, and pulled taut using straps, belts or draw strings for tightening the garment over the patient body. It may also be adapted for non-human anatomy for use with veterinary medicine, or with other general objects. The garment 170 may possess an electronic x-ray source, and/or one or more x-ray detectors.

In some embodiments, the garment may be used to view and/or treat the interior of a patient (human or animal). In another embodiment, the garment may also be used on a parcel, bag, luggage or other object to view its contents non-destructively, for example, in conjunction with the devices, systems and methods described herein.

In some embodiments, the UID 152 may be wirelessly connected to the control unit 102, or a backend computer system, or connected to the cloud (FIG. 1B). User interaction information (e.g. touch controls, gestures, sensation, the ‘feel’ of traction when manually handling the proximal end of a medical device) to the UID can be relayed to a control unit or computer or other electronic device wirelessly using any medically acceptable wireless protocol.

In some embodiments, there may be three sources of image data for the system and methods to generate the enhanced reality image (FIG. 1C). In an embodiment, the patient may begin with a scan of internal anatomy using an internal image scan device, such as a computerized tomography (CT) scanner, magnetic resonance imaging (MM), ultrasound (US) or other imaging system. CT scans are frequently referenced herein, however the systems, devices and methods described are intended for use with any internal imaging system. The use of “CT scan” or “CT scan data” is therefore not limiting only to CT scans, but inclusive of all imaging technologies currently used or to be used in the future. CTA may refer to computer tomography angiography. The internal image scan device while not part of the system described herein, can be a first step in the treatment of a patient. The patient P may lay in a position to be scanned. The patient may have a contrast agent as part of an IV or intra-arterial or intra-muscular or endo bronchial or any other solution 160 that is currently used or may be used in future to highlight targeted anatomy during imaging. The patient may wear a radio-visible (opaque, semi-opaque, or air filled) marker, such as a fiducial marker F. Once the CT scan is completed, the patient has a sensed tool 162 inserted into their body P. The sensed tool can be tracked using the systems and methods described further herein. The sensed tool position data can be mixed with the patient images from the CT scan, and visual images from one or more cameras 180, 182. In this process, there may be an electromagnetic signal cable 164, and EM transmitter 104, a sensed tool 162, a wearable display 150 having one or more cameras 180, 182 for the HCP, and one or more EM markers in the sense tool and/or fiducial marker. The tool tip can be inserted into the patient and used to cross a lesion L while the visual representation can be provided to the HCP through the glasses 150. In various embodiments, traditional fluoroscopy may be used at any time. Fluoroscopy may be used to enhance or further define the position of blood vessels or organs and may be used by the system to provide additional 3D/4D mapping data. Users may also use fluoroscopy during a medical procedure where the enhanced reality map is well established, to lend additional confidence to the user or help the user orient themselves to the enhanced reality image.

In another embodiment, data from a pre-operative computed tomography (CT) angiography (CTA) scan 130 may be combined with visual image scans of a patient P using one or more fiducial markers F on or in the patient (FIG. 1D). The fiducial markers F can be used to provide location reference points to correlate the visual scan data of the patient, whether that visual scan data is of the exterior of the patient P body, or aspects of the patient P interior (e.g. arterial system, venous system, heart, kidneys, etc.). Visual scan data may be captured using one or more video camera(s), X-ray devices (i.e. fluoroscope), ultrasound imaging, positron emission topography (PET) or other imaging modalities. In an embodiment, a minimally invasive device, such as a sensing probe 120 may be inserted into a patient P and used to provide image data of a particular region of the patient body. The image data from the minimally invasive sensing probe 120 can be correlated with other available image or topography data to provide a computer-generated image to a user. The computer-generated image combining two or more available data types can be used to create a virtual reality (VR), augmented reality (AR) or enhanced reality (ER) of the volume of space the health care provider is interested. This targeted volume of space may be a disease area, injury area, or simply an area the system generates an image for as the sensor moves through the body.

In one non-limiting embodiment, a minimally invasive sensor probe 120 may be advanced into a patient through the groin. The device may be advanced through the arterial system following the natural path of blood vessels to the aortic arch. The sensor probe may be an electromagnetic sensor, a micro x-ray emission device, a nuclear imaging probe, an infrared imaging probe, or a non-invasive imaging or sensing device. In another embodiment, a sensor can be a micro x-ray emission device, an x-ray detection film (or electronic x-ray detector) can be positioned outside the patient body and a desired location. The micro x-ray device may be remotely activated so a small dose of radiation will illuminate the detection plate and produce a controlled, targeted and lower radiation exposure than traditional x-ray imaging. The image produced can be used as a still, or a series of images can be taken continuously or at some interval of time, to produce a series of images. These images may be used alone for x-ray images of the targeted area, or in combination with other image or sensor data in an integrated image modality.

In some embodiments, the data analysis and integration of multiple imaging modalities may be done in a control unit 102. In other embodiments analysis and integration may be done in a backend system that can be located remotely from the area where the patient procedure can be carried out. In still other embodiments, the analysis and integration may be done by cloud computing. In some embodiments, the control unit may gather data that may be cloud based or remotely located. Data may be collected and utilized in the planning of current or future diagnosis, medical procedures and treatments. Images and data may be displayed on goggles 150 at any time. The goggles or glasses 150 may also have at least one camera 180 for capturing visual images of whatever the wearer may be looking at. In some embodiments, image and/or data may be displayed on goggles when a care giver first meets with a patient. The care giver may see the patient naturally through the goggles. The goggles may be made of a transparent material having a portion of the goggle lens adapted for displaying virtual reality material. In some embodiments, the goggles may be made from a material that is partially transparent to visible light (i.e. organic light emitting diode (OLED) display) so virtual images (optionally including data) can be displayed on the goggles while a user can still see through the material at whatever might be in front of them. In various embodiments combinations of materials may be used for the goggles including OLED, light emitting diode (LED), liquid crystal display (LCD), polarized glass (or other polarized transparent materials). Further, in some embodiments, the goggles may be made of more than one kind of optical and/or display material. In some embodiments, the goggles may have an audio, and/or a tactile sensing and feedback component as well. In yet another embodiment, the goggles may have electronics that communicate with one or more devices implanted in/on the patient or the HCP. This communication may be completely wireless, asynchronous (without prompt) or synchronous (on demand) during a physician visit or a procedure or a post procedure visit.

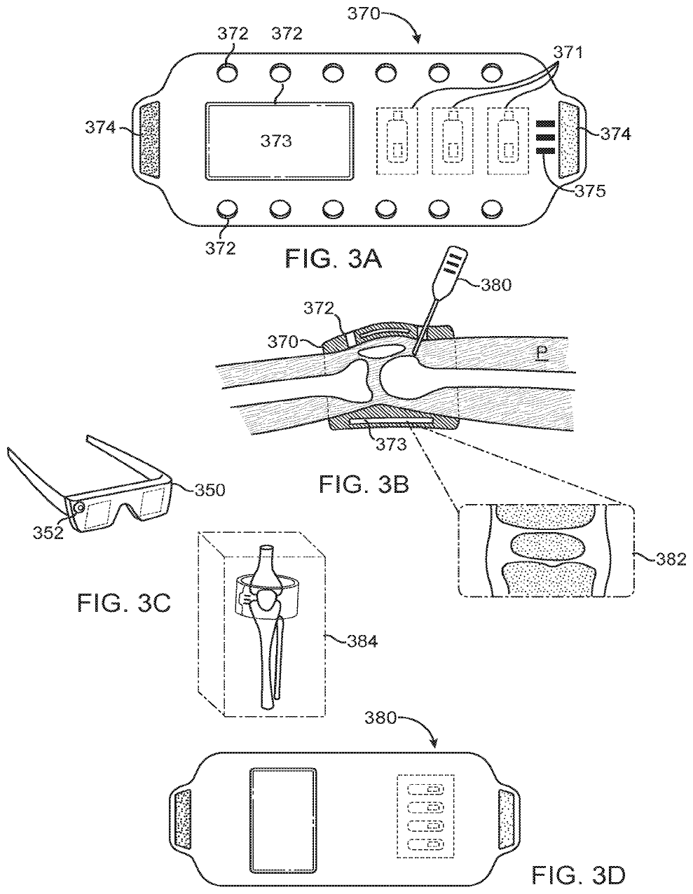

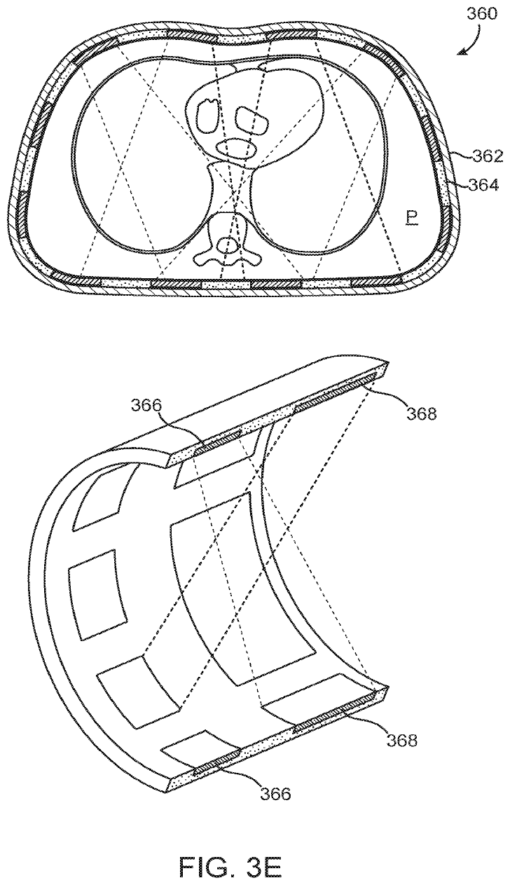

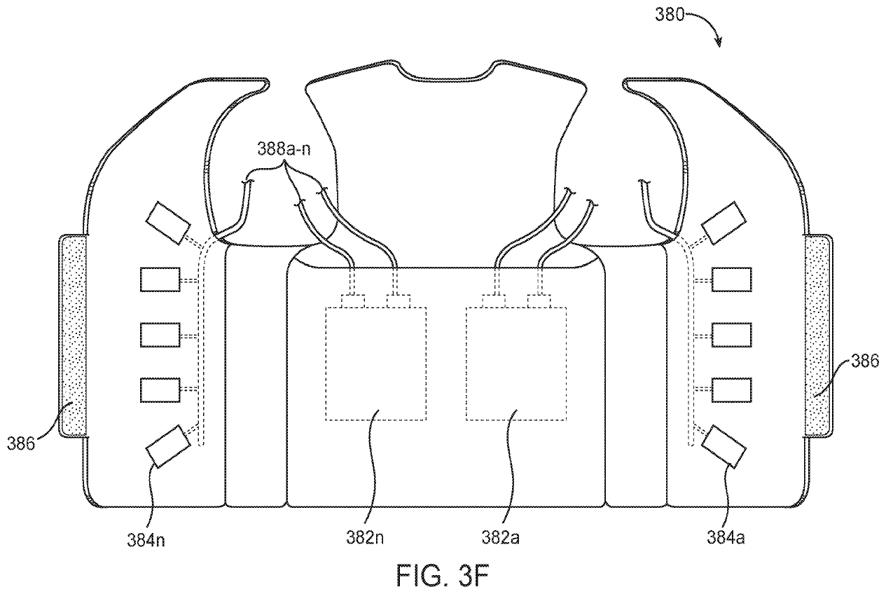

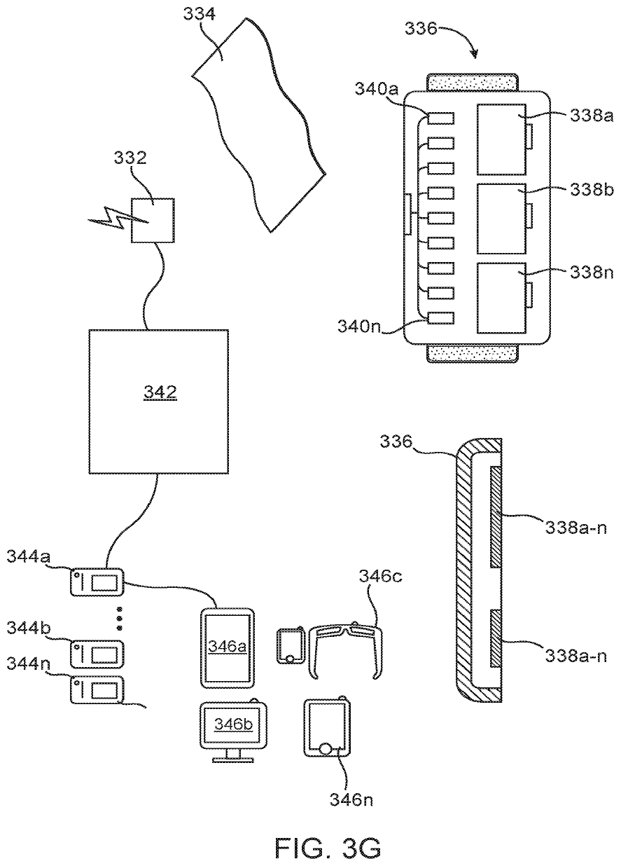

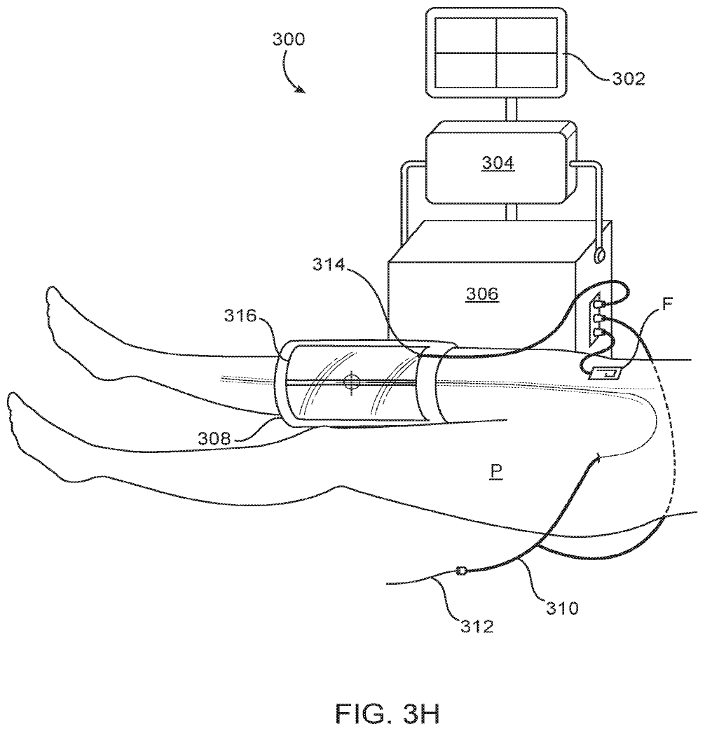

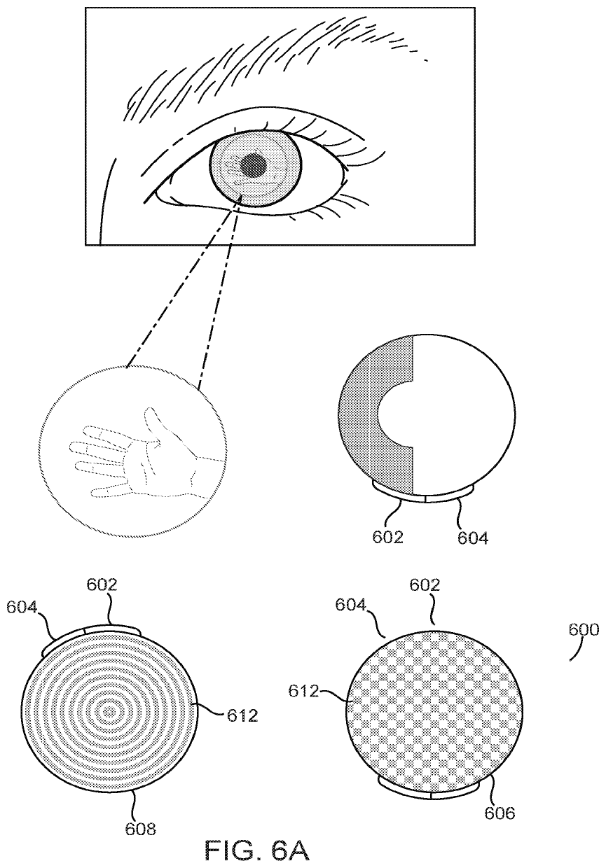

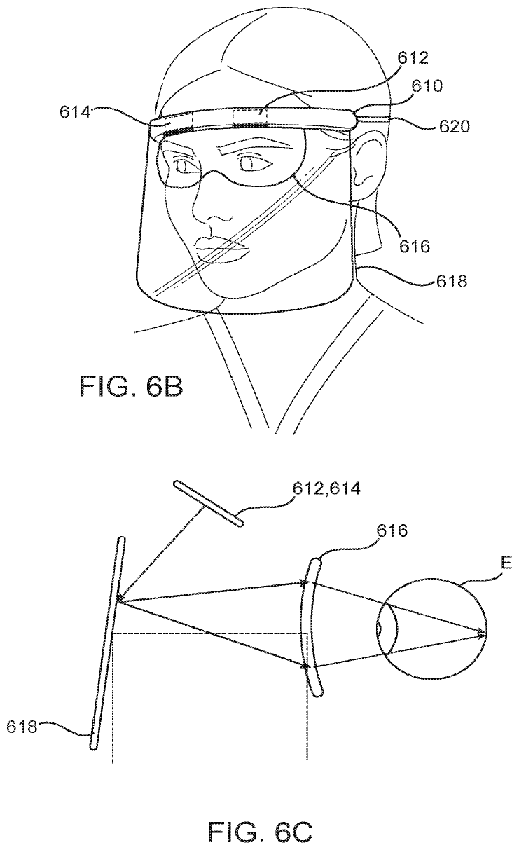

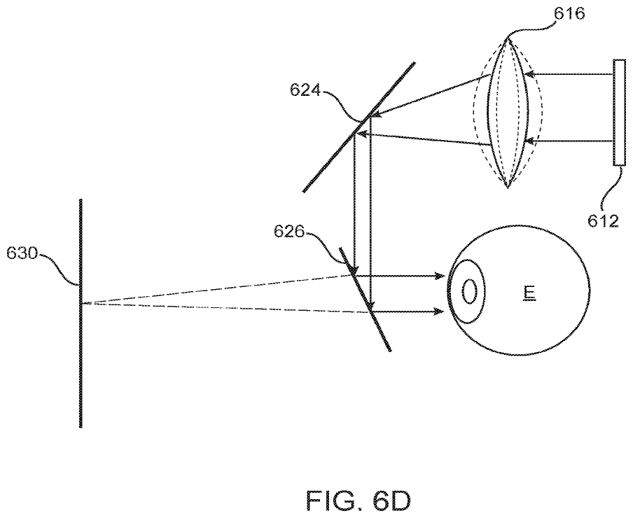

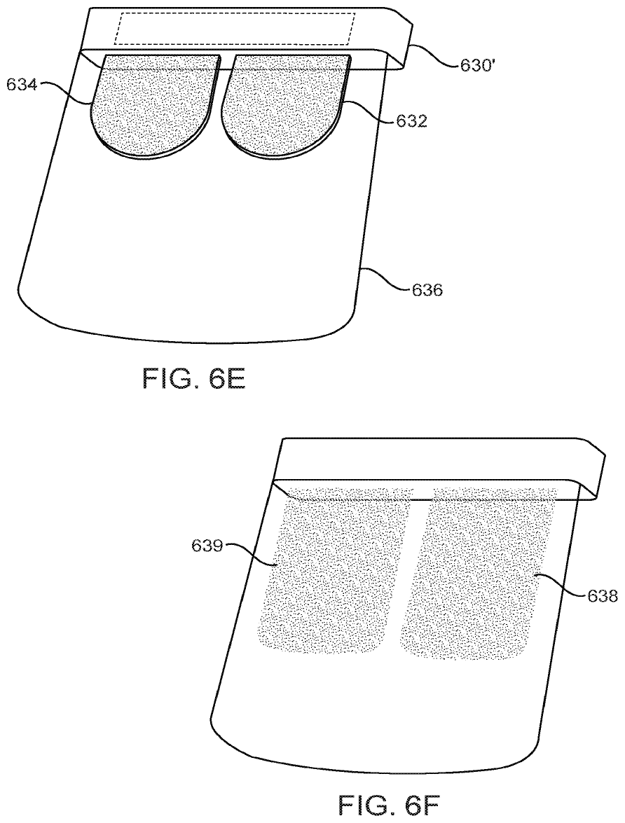

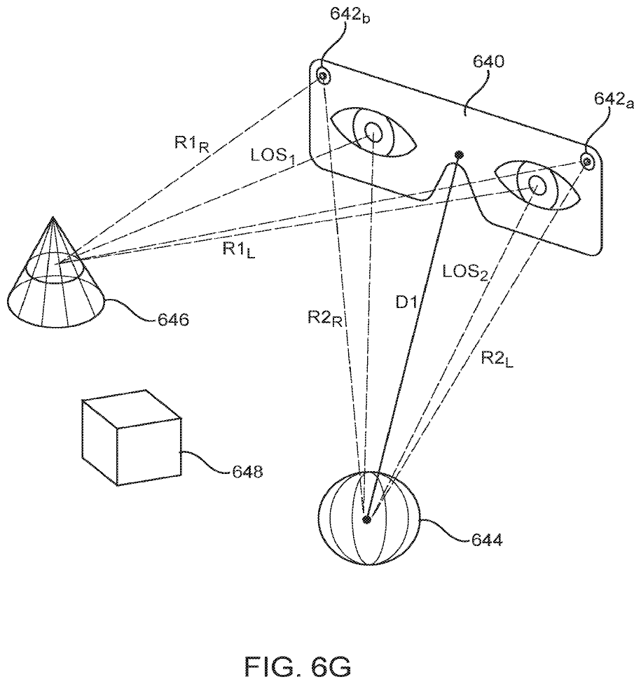

In another embodiment, the Enhanced Reality Display of the goggles 150 may be a true enhanced reality holographic medium (ERHM), disjoint from the goggles themselves. This ERHM may be a physical 2 or 3 dimensional active or passive display of enhanced reality images in a way that the images accurately superimpose on the object(s) behind ERHM. In an embodiment, an ERHM comprises a (semi) transparent film that is otherwise not visible, unless enhanced reality images are projected right on it. In another embodiment, an ERHM may compose of a semi-transparent mesh of programmable display elements. In yet another embodiment, an ERHM may be composed of a virtual floating region signaled or held by a user's gesture. In yet another embodiment, an ERHM may be a temporary physical dome or enclosure or a flat display (FIG. 3E) that appears between the user and the object(s) on demand to display enhanced reality images and then moves away. In yet another embodiment, an ERHM may comprise of a transient nebulous (cloudy) material (FIG. 6F, 638) that lets normal light through but partially blocks (and thus displays) a special kind of light projected from goggles 180, or another projection medium.

In various embodiments, the correlation of the various data images as described herein may rely on at least one frame of reference for all the image data, wearable display orientation and other position references required. In some embodiments, the frame of reference may be made to one or more origin points. In some embodiments, the origin point(s) may be the position of the fiducial markers placed on the patient. The position of the fiducial markers can be the same for all the image scans taken of the patient regardless of the modality of image sensing. If the fiducial positions are the same for each image sampling, then the function of correlating the various image data may be simplified. The origin reference may be a position triangulated from the fiducial positions, or the system may use a point of origin that can be fixed in space. In some embodiments, the room where the patient rests may have a fixed origin generated by a localized position tracking network. In some embodiments, the reference frame for each image may be different from the reference frame of each other image. In such an embodiment, each image may be independently correlated from each previous and each successive image. In still other embodiments, each image may use a base averaging correlation routine where the correlation of each correlated image can be guiding in the correlation of position and image date for each successive image, but the algorithm may ignore the averaging of previous data correlations to derive a new correlation for any particular image and position set. A position tracking network may use visual, wireless or audio signals to determine the location of various other objects in the room. The position tracking network may operate like a room sized global positioning system (GPS) where the room (or area of patient treatment) is the globe.

In one non-limiting example, the pre-scan data 130 and the fiducial position markers F may be correlated using a gating capture technique. As the internal organs are scanned, the patient may be asked to hold his or her breath at a regular interval. For example, the patient may be asked to hold their breath right after a long breath or a sensed heart beat and a single layer of imaging be done. In this way, the imaging introduces the least artifacts due to the patient voluntary and involuntary movements. The fiducials help correlate the external structures with the position and orientation of the internal organs since they are present during the entire scan. Later, when other imaging may be done, a similar gating process can be used so the margin of error in the second and subsequent scans shares, as much as possible, the same artifacts as the first scan.

In some embodiments, the fiducials may be registered with the control unit using an optical system. In some embodiments, the fiducials may be electromagnetic markers and registered using RF or other wireless energy. In some embodiments, the fiducials may each emit a different frequency of sound that can be picked up and registered with the system. The system can use the EM field generator for registration of the fiducials. In some embodiments, the goggles may be used to register the fiducials. In some embodiments, an additional component (not shown) may be used to register the fiducials.

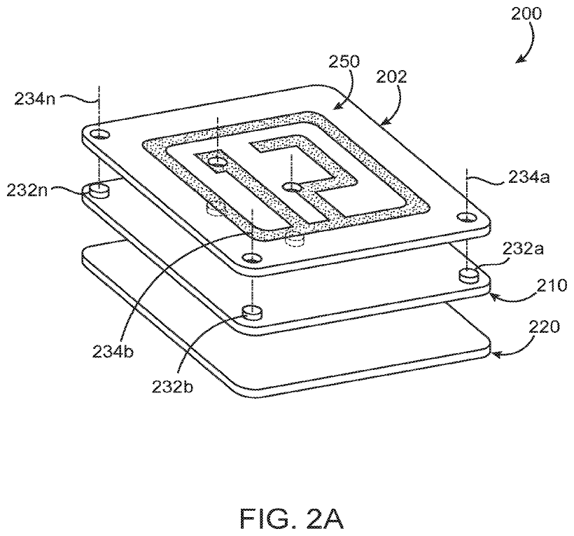

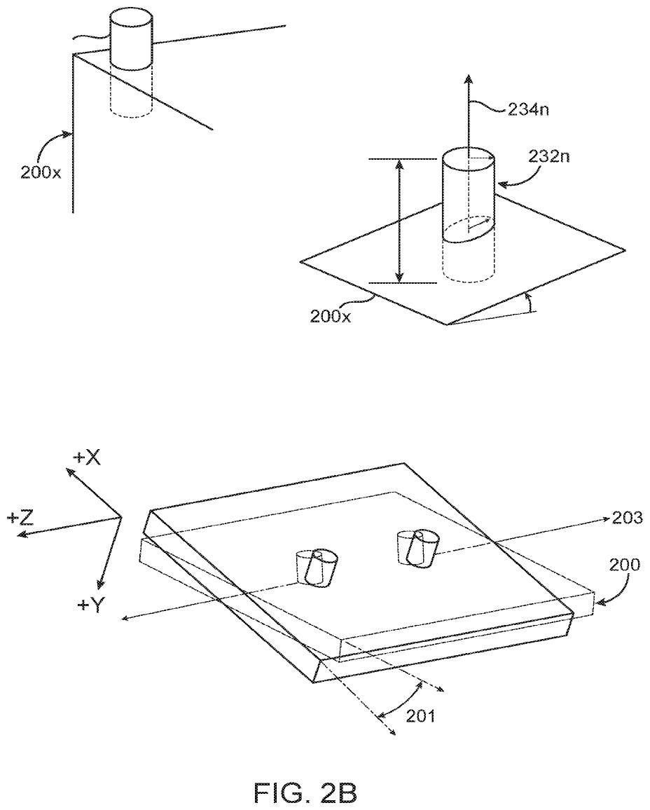

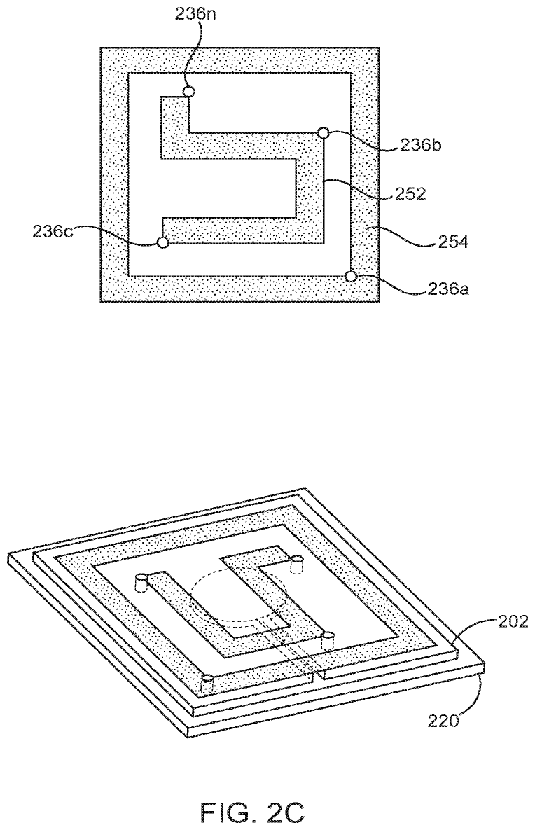

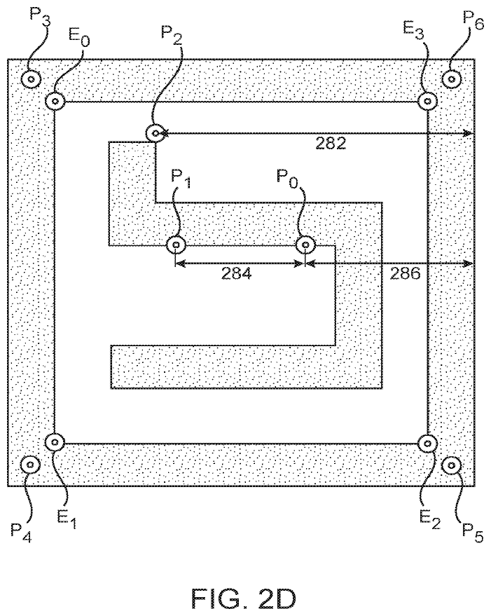

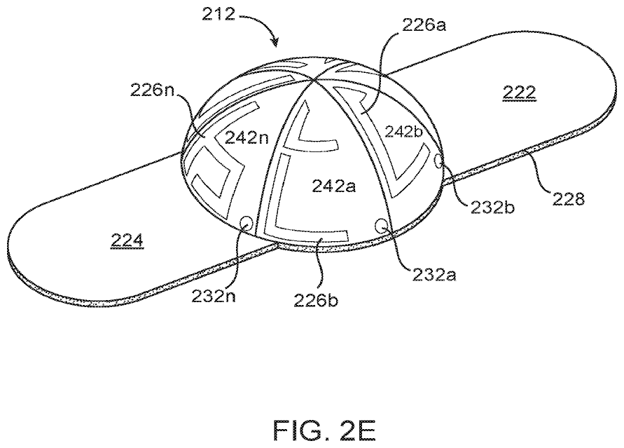

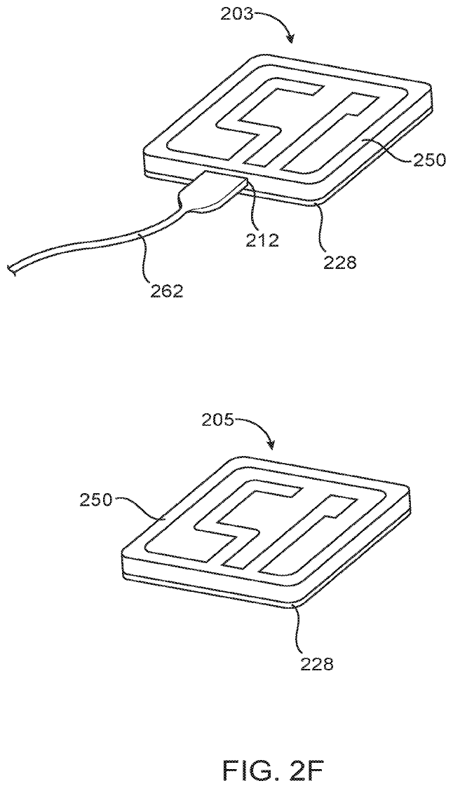

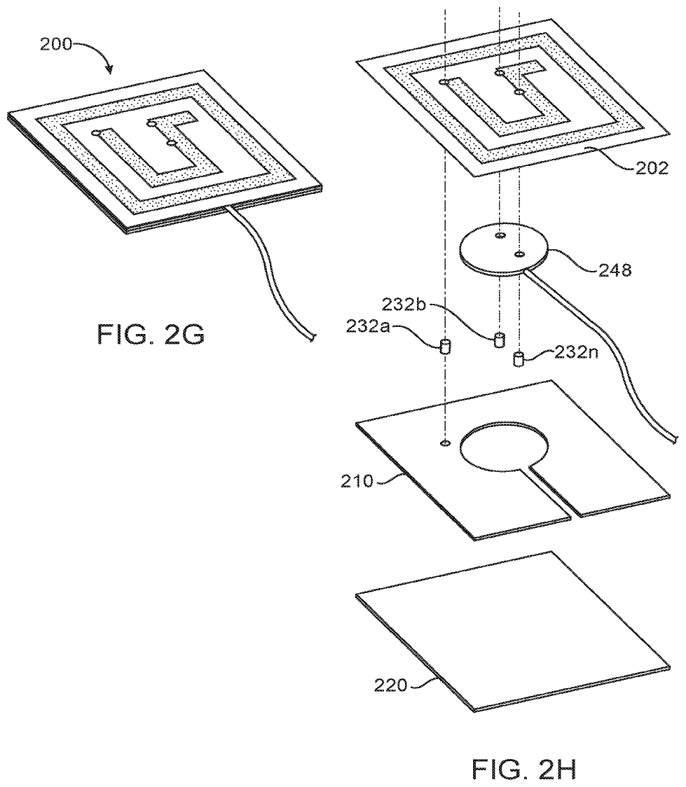

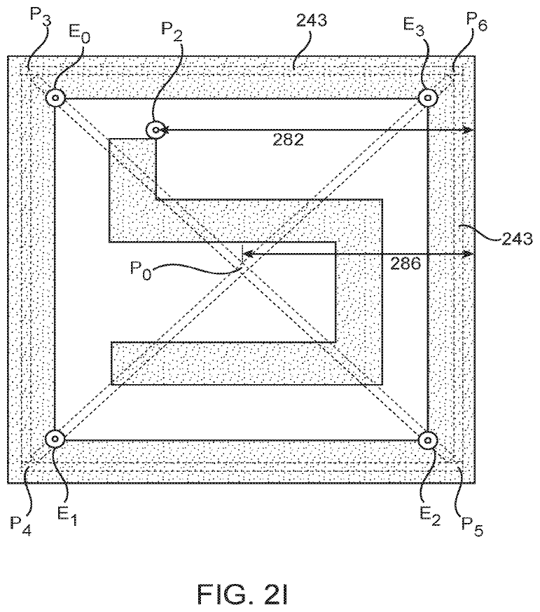

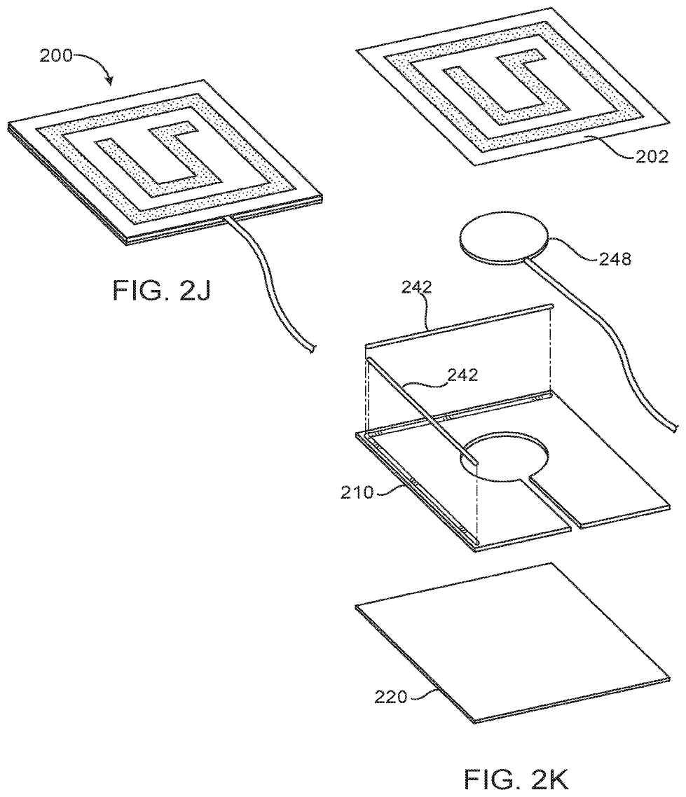

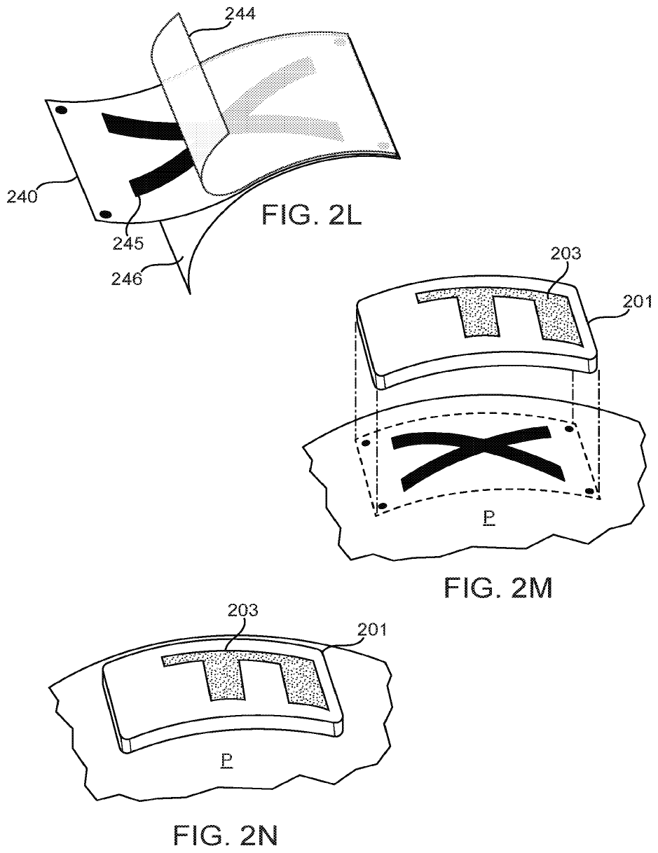

In some embodiments, there may be a fiducial marker 200 (FIG. 2A). The fiducial marker may have several layers, such as a top layer 202, middle layer 210, and bottom layer 220. Note the assignment of top and bottom may be completely arbitrary. The side facing up (alternatively the side visible to a user) is generally referred to as the “top.” Fiducial prints may be made on any and all visible surfaces so any visible surface may be the “top.” This includes a narrow edge surface, which one can image would be facing up and be the top, if the fiducial marker was placed on a patient's side so the larger surface area side was facing a generally horizontal plane. The fiducial marker 200 may have one or more visual fiducial prints 250 on its top face. The fiducial marker may also have one or more sensor detectable devices 232—embedded in the fiducial marker. Each sensor detectable device has an axis 234—of alignment. Note the reference to a part with the subscript “n” refers to a part that may be repeating any number of times so the determination of an exact number of the part is difficult to precisely state. Here the sensor detectable device can be any material or electronic device that can be detected by an electromagnetic sensor(s). The sensor detectable devices can be in various shapes and sizes, and can either broadcast their own signal, or respond with a signal when pinged. In some embodiments, the sensor detectable devices may be completely passive, and are simply registered in time and space when an electromagnetic sensor sweeps the volume of space the sensor detectable devices are in. The sensor detectable devices (SDD) may provide information to the electromagnetic sensor in the form of the SDD's position, orientation, size, composition, shape, volume, mass, batter state, or any other information desired. Multiple SDDs may be positioned at various places in the fiducial marker, providing a greater number of SDDs for an electromagnetic sensor to detect, and get higher fidelity than from tracking a single SDD.

In some embodiments, the SDDs 232n may be positioned in the fiducial marker 200x, or protruding from the fiducial marker or affixed to the surface of the fiducial marker 200x (FIG. 2B). In some embodiments, the alignment of the SDD may be normal to the plane of the fiducial marker 200, and in some embodiments the SDD 232n may be at an angle 234n to the plane of the fiducial marker 200x. The fiducial marker 200, 200x may move in three dimensions during the course of a medical procedure, and the movement of the fiducial print 250 and SSDs 232n can move in various ways. In one non-limiting example, the fiducial marker 200 can rotate on an axis 203 defined by a pair of SDDs, and the outer edge can move by an angle 201. It should be appreciated that as a patient breaths, or moves for any reason, the fiducial marker 200, 200x will also move by an amount corresponding to its placement on the patient body. X, Y and Z axis are illustrated simply for reference. The presentation of the three standard axes is not meant to indicate the arbitrary coordinate origin of a three-dimensional space. In some embodiments, the fiducial marker may have a port or aperture for the insertion of a medical device or instrument through the marker. Allowing a user to use the fiducial marker as an access point to the patient allows for enhanced tracking of the entry point of the instrument into the patient, since the surface and position of the fiducial marker can be accurately determined.

In some embodiments, there can be a multilayer fiducial marker (FIG. 2C). One side of the fiducial marker may have a visual print 252 and a visual border 254 that can be detected by an optic scanner (camera, pattern recognition device, laser scanner/barcode reader or other system). The visual print or optical image may have a particular shape to designate a direction (such as “up” or “inward” or “outward” relative to a patient body). The optical image can have one or more points 236ₐ, 236_{b}, 236_{c}, 236ₙ anywhere along the image or surface that are encoded to provide additional information. The point information 236ₙ on the surface may have known distances between them, so when read by an optical reader or scanner, the distance between the points in the image can be compared to the planar distance between the points on the marker. A calculation can be used to determine if the marker is at an angle to the camera/optical reader and determine the angle of the marker. The points may also contain additional material, such as radiopaque markers (i.e. a lead bead), so the marker can be scanned with an image transmission scanning device (like an x-ray machine). The marker may have layers of material. Embedded within the layers (or on one of the surfaces) may be a cutout designed to seat an additional sensor in a fixed position and orientation to provide additional sensing data during a procedure, registered with the marker's frame of reference. The marker may have a modular design that will allow for a marker without an extra embedded sensor to be imaged (CT, MRI, Ultrasound, or a similar modality), and the extra sensor inserted in only one allowable way in the marker prior to an actual procedure (This may allow for extra sensor elements potentially with cables to be inserted when needed without causing inconvenience to the patient). One of the marker layers may be adhesive, or have an adhesive component, to allow fixing the marker onto the patient's skin or body. In an aspect, the marker may be square, between 50 and 80 mm on each side and between 5 to 10 mm thick. The marker may have a channel for receiving an insert for a scanner or detector. In another example marker may be 100 mm on a side and 10 mm thick. In still another embodiment, the marker may be any shape and size so long as the visual print can be read. The distance to the fiducial marker may be measure using an infrared sensor, laser range finder or other technique. An electromagnetic sensor may also measure the distance from the sensor to the fiducial marker, and correlate a known distance between an observation camera to determine the distance of the fiducial marker to the camera. Some of visually discernible features on the marker's surface may be made of special material that can be readily identifiable by a camera device at a specific wavelength. The special material may also be an active fabric that displays programmable features unique to the patient or procedure, and may change detail depending upon the specific needs of the procedure (e.g. less or more accuracy). Further, the marker may have one or more miniature camera embedded in it. Such a camera may assist to capture the operating field from the patient's point of view, track the position and orientation of HCP, or help provide better estimation of it's distance from the HCP, accuracy of correlation. This marker embedded camera can also be used to sense the focus and direction of the HCP's gaze by directly observing him/her from the marker's vantage point.

In another embodiment, the marker may serve as a display for cues or patient vital information at certain points in the procedure. The marker's boundary may have a strip that changes color based on the level of accuracy of correlation during the procedure. In one non-limiting example, the marker strip may change from normal to green for less than 1.0 mm average error, or yellow for 1.0-2.5 mm error, or red for error margin greater than 2.5 mm. The marker may have simple indications to guide the HCP in driving the interventional device in a certain direction, such as turn left, or turn right, or advance slow, or advance fast; all as non-limiting examples.

In another embodiment, miniature carbon nanotube based x-ray imaging sources may be embedded in the marker, with a detector on the other side of the patient (on the procedure table). The captured image of the interiors of the patient's body may be sent to the data processing component to be merged with the combined Enhanced Reality Image for live guidance.

In another embodiment, a variety of defined sensor positions are identified throughout the fiducial marker (FIG. 2D). The fiducial marker may be defined with X and Y coordinates and the position of various types of sense-able elements (elements that can be sensed by various sensor devices, or they may be SDDs) are positioned around the face of the marker. The chart below provides position data for one non-limiting example of placement of sensor detectable devices.

| Order | Identifier | Coordinates |
|---|---|---|
| 3 | P₀ | 0, 0, 0 |
| — | P₁ | −14, 0, 0 |
| 1 | P₂ | −20, −17.5, 0 |
| — | P₃ | −43, −52.5, 0 |
| 2 | P₄ | +45, −52.5, 0 |
| 4 | P₅ | +45, +37.5, 0 |
| 6 | P₆ | −43, +37.5, 0 |
| 7 | E₀ | −39, −47.5, 0 |
| 10 | E₁ | +41, −47.5, 0 |
| 9 | E₂ | +41, +32.5, 0 |
| 8 | E₃ | −39, +32.5, 0 |

In some embodiments, P (patient) markers may have position sensors (like SDD) embedded at their locations. They may also be seen in patient internal image scans and are used to correlate internal image scan data with actual patient marker positions using position sensor readings. P markers are not required to be visible to camera and can be embedded within the fiducial marker layers.

In some embodiments, E (Enhanced reality) markers can be feature points that can be visible to the visual image camera (tablet, fixed camera, glasses/goggle mounted camera, etc.) and connect visual image with the scan image data. E markers may be visible to the visual image camera. The relative position of the E and P markers are used to determine the various positions of objects relative to the markers, thus the position of the P and E markers relative to each other is known. While the E and P markers are shown here as discrete points, there is no requirement that the E and P markers have a specific shape, orientation or position. The E and P markers may be dots, short lines, small shapes or any other geometry so long as the shape, position and size of each E and P marker are known to the system, and the system can accurate determine the relative position of each E and P marker relative to enough of the other E and P markers to make the system work.

In some embodiments, the system may utilize all the E and P markers in the fiducial marker. In some embodiments, the system may use only a portion of the E or a portion of the P markers.

In addition to the coordinate position of the various P and E markers, there can be a fixed linear distance between various elements, such as the distance between the center of P₁ and P₀ 284, the distance between P₀ and the edge of the fiducial marker 286, or the distance between P₂ and the edge of the fiducial marker 282. It can be appreciated that any distance between any two points can be used.

In still another embodiment, there may be a marker design for collaborative enhanced reality experience (FIG. 2E). This marker may allow multiple users to experience the same enhanced reality sense as the operating physician. The marker has a circular or dome center section with two tabs extending outward, the tabs being generally opposite each other. In an embodiment, one tab may extend toward the medial side 224 of the patient while the other tab extends toward the lateral side 222 of the patient. The marker may also have an adhesive backing 228 for firm placement on the skin of a patient. The center circular area may be divided into wedges or sectors 242ₐ, 242_{b}, 242ₙ. Each wedge may have a distinct visual print or marker 226ₐ, 226_{b}, 226ₙ, and a SDD 232ₐ, 232_{b}, 232ₙ. In operation, the dome shape of the fiducial marker allows users standing around the room to use their individual goggles or glasses with a video camera. Each camera will see the fiducial marker facing them on the dome and allow the system to track their distance from the dome, the direction they are from the dome (by viewing the distinct visual print 226ₙ they can see, and do an independent correlation of user position to patient position, and correlating all relevant data for each individual user so each user is provided with a proper perspective of the procedure. Each sector may correlate to the same planning images through geometrical constraints. In some embodiments, the collaborative enhanced reality experience marker 212 may have an embedded microphone and camera to take audio-visual commands from HCP, example: “focus 1 mm deeper” (or an associated pre-programmed visual gesture) or “show me a close-up of lesion” (or an associated pre-programmed visual gesture). These commands may then be relayed to the control unit and the enhanced reality display adjusted accordingly.

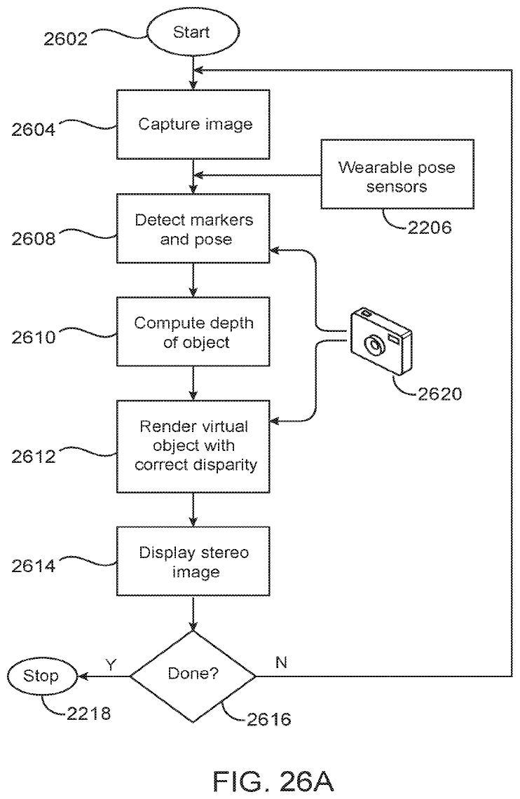

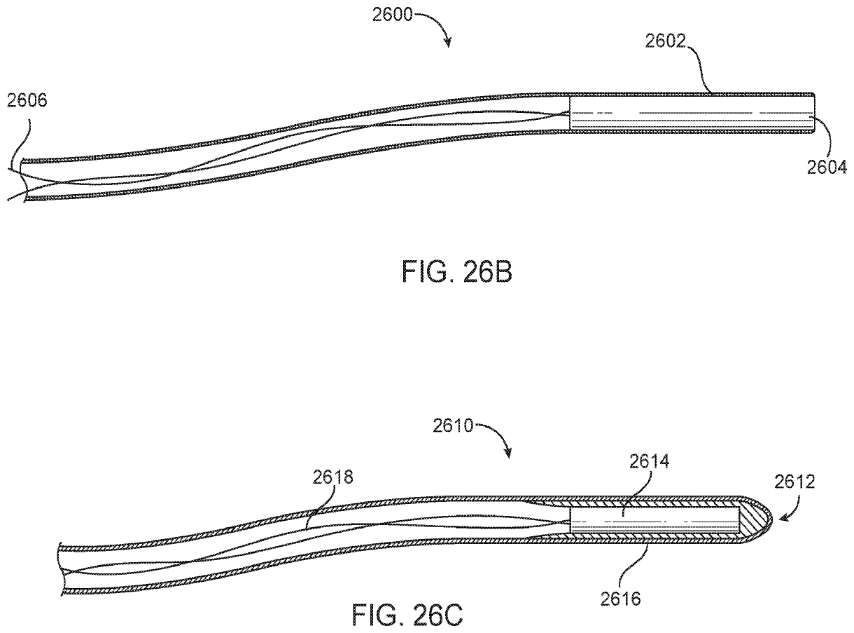

In some embodiments, the fiducial marker 203 may have an access port 212 (FIG. 2F). The access port 212 may connect a medical device through a cable 262. The fiducial marker 203 may have some electronics so it can receive and process signals from the medical device cable 262. The medical device may be any kind of medical instrument, device or tool having one or more SDD that can communicate information to the electronics on board the fiducial marker. The fiducial marker with electronics has a visual print 250 that may be seen by a camera. In an alternative aspect, the medical device may communicate with a fiducial marker 205 via a wireless communication protocol. In some embodiments, the medical instrument may be a guidewire 2600 having a SDD 2604 placed at the distal end of the guidewire 2600 (FIG. 26A). The guidewire 2600 may have a sheath 2602 and electronic communication wires 2606 which may connect to a computer controller, or a fiducial marker.

In another aspect of the fiducial marker, an exploded view is provided showing the fiducial marker 200 (FIG. 2G) with a top layer 202, middle layer 210 having a shaped aperture for receiving a disk-shaped sensor 248, and a bottom layer 220 (FIG. 2H). A group of SDD can be placed within the fiducial marker, and as can be seen, one SDD is seated within an aperture in middle layer 210 while two SSDs are position to sit on middle layer 210. This allows one SDD 232ₐ to be seated at a different depth from the others 232_{b}, 232ₙ so the three SDD form a three-dimensional pattern in the placement within the fiducial marker. Using a three-dimensional placement can improve fidelity of identifying the position of the SDD, and produce a higher resolution image, or higher resolution image data file. In an embodiment, the disk shaped sensor 248 may assume any other general shape, and may have holes in it in a different configuration that shown in FIG. 2G. In yet another embodiment, 248 may have visual imprint features directly on it, to allow its use in conjunction with 200 or by itself, depending on the level of accuracy desired by a medical procedure.

In some embodiments, the top layer, or the side having the visual print may be removable, and substituted with a different visual print. The replacement of the visual print may allow for higher resolution of the visual image, and higher resolution of the various image maps and coordinates derived from the higher resolution visual print. Any replacement of the visual print can be done with knowledge of the resolution and possible changes in position data relative to the visual print compared to the internal SDD elements. In yet other embodiments, different parts of the visual imprint may have different optical properties to improve the accuracy and robustness in detecting them with a sensing or detection system. The differing optical properties may include, but may not be limited to: reflectivity, frequency response, refractive index, specularity, and emissivity.

In some embodiments, the SDD may be a strip or rod placed in a pattern under the visible print of the fiducial marker (FIG. 2I). The SSD material may form a pattern of a known geometry, and the system may have dimension information of each piece 243. In this embodiment the entire rod or strip can form the P position, and instead of a discrete point, the P position can be a line, bar cylinder or other shape. The relative position between the P reference and E reference markers are known to the system, regardless of the shape of the P and E markers (the E markers may also be various shapes and sizes (not shown)). The system may use the known length, width, thickness or other values of the SDD pieces 243 to calculate the position of elements in the internal image scan. In addition to the dimensions and/or characteristics of each SDD piece 243, the system may track the angle between the SDD pieces, angles between the SDD pieces and edges or positions of the visual print, or between the SDD pieces and the edges or other features of the fiducial marker as a whole.

In some embodiments, the fiducial marker may use a continuous rod or strip of material that can function like a SDD (be detectable to a sensor or imaging device) instead of discrete bullets or pellets (FIG. 2J). An exploded view is provided in FIG. 2K. In such an embodiment, the dimensions of each rod or strip are known. There may be 2 or more such continuous rods placed at an angle to each other. The length of each rod and the angle of connection can be known, so the geometric position of each rod relative to the visual aspect of the marker can be used to help calibrate and determine the position of internal elements from the sensed image data.

In another embodiment, the fiducial marker may be a two component device. In one aspect, the fiducial marker with the SDD component may be a flexible stick on sheet or a temporary tattoo (FIG. 2L). The temporary tattoo can have a SDD marker in the form of an “X” or as a series of discrete dots, mimicking the pattern of the SDD markers described herein. The stick-on or temporary tattoo can be placed on the patient skin by a user. A sterile barrier 244, 246 can be removed prior to placement. If the sheet 240 holds a temporary tattoo, the image is transferred to the patient. If the sheet 240 is a stick on, then the sheet simply adheres to the patient skin or body surface. Once the sticker/tattoo is in place, the patient can be scanned using an imaging modality (x-ray, CT. MM, or the like) and the scan image data with the fiducial markers are recorded. After the image data is acquired, the patient may be prepped for a minimally invasive medical procedure, which may be the same day, or a day or more after the image scan in taken (so long as the sticker/tattoo is still in place when the medical procedure is to take place). When the patient is prepped for the medical procedure, the visual print aspect of the fiducial marker is lined up to the sticker/tattoo on the patient body, and placed on top of the sticker/tattoo (FIG. 2M). The use of the visual cues (dots) in the corner of the sticker/tattoo can be used to align the visual print on top of the SDD marker. Once the visual detectable feature is in place, the procedure may continue as described herein (FIG. 2N).

In various embodiments, any fiducial described herein may have a communications port for direct physical access to an electronic cable. Such electronic cable may be connected to a medical device, a computer, a sensor or a wearable device.

In another embodiment, an example sensor garment 370 is shown (FIG. 3A). The example sensor garment 370 shown is a band that can be wrapped around a body part such as an arm or leg. A larger band may be used around the chest or head. Alternatively, the garment 370 may be a vest for use on the chest. The sensor garment has a detector 373 for receiving x-rays or other electromagnetic energy. In some embodiments, the electromagnetic energy may be nuclear imaging signals. In still other embodiments, the sensor garment may have detectors for chemicals, bio-molecular materials or mechanical energy. The detector may also be a transducer for receiving electromechanical energy such as ultrasound waves. The detector 373 can be set up on the interior side of the sensor garment 370 so the detector 373 is adjacent and/or touching the skin when the garment is placed on or around the patient body. In some aspects, the sensor garment may need a coupling agent, such as an ultrasound coupling gel, water or other material. The sensor garment 370 may have one or more optional energy emitters 371, such as x-ray emitters. These x-ray emitters may be micro sized x-ray seeds, or electrically powered x-ray emitters. The sensor garment also has one or more openings or apertures for exposing the patient body through the sensor garment. These openings may be used to deploy medicine or other medical instruments to the patient body beneath or enclosed by the sensor garment. The sensor garment may be secured in place by using a fastener 374, such as a clip, buckle, a removable sticker, or Velcro® strap. The sensor garment may also be just left hanging on the patient body using gravity or an external support in cases of trauma or emergency imaging where contact with the patient is not advised. The sensor garment may have one or more optional fiducial markers 375 with visually or indirectly detectable features.

In some embodiments the detectors are able to detect and properly determine the angle of the energy emitter, determine if the energy received is a primary intensity (received directly from the emitter) or a secondary intensity, such as if the energy is reflected or refracted from something in the volume being imaged. In some embodiments, the image detection may be from any angle.

In an aspect, the sensor garment 370 may be wrapped around a patient knee (FIG. 3B) and a point source x-ray device 380 may be inserted into the patient through one of the openings 372 in the sensor garment. The point source 380 may be placed adjacent the area of interest and aimed so its radiation will project toward the detector 373. In this fashion, a specific location can be imaged using the desired imaging modality with a minimum exposure to health care workers or the patient to excess or stray radiation. In another aspect, the point source x-ray device can be a part of the sensor garment, located so it may allow imaging of the anatomy the garment wraps around, onto one or more detectors on the other side of the anatomy. In some embodiments, the emitter and detector may not be on opposite “sides” of the body. In some embodiments, the emitter may be placed in close proximity to the detector and the path through the body between the emitter and detector can be a chord (joining any two points along the circumference of the body outline). A specific target image 382 may be produced that can be incorporated into other patient data to provide an enhanced reality view of the work site. In other embodiments, the sensor garment may also serve as a ‘patient stabilization device’ to hold the patient site in a specific pose during imaging, as determined by the medical treatment plan; and also be able to reproduce the same pose during treatment or intervention to minimize correlation errors. In an embodiment, the enhanced reality images generated from the pre-operative scan (CT, MRI or similar) may also include the silhouette of important large body parts, to assist in ‘recreating’ the pose the patient was in during the imaging. This view may show the scanned pose and the real pose as body silhouettes overlaid on top of each other, and guide an HCP or the clinical personnel to match the two to an acceptable clinical accuracy level before starting the procedure. A score of gross body silhouette match may also be displayed to the HCP or clinical personnel to guide them with patient positioning. In some embodiments, any part of the body may be imaged using either a wraparound sensor garment or the sensor garment may lay on top of, or be affixed to a soft tissue area for scanning. Some non-limiting examples of soft tissue areas include the nose, breast, ear, penis, or areas of enlarged adipose tissue (gut, thigh, buttocks). In various embodiments, the enhanced reality imaging capabilities may be used for diagnostic purposes.

In another aspect, the image data 382 may be used as part of an integrated image modality to produce a three dimensional (3D) or four dimensional (4D) scan of the desired work site (FIG. 3C). The integrated image 384 may be viewed on a tablet, computer screen, an Enhanced Reality Holographic Medium or displayed on goggles/glasses 350 having computer image projection capabilities. The goggles/glasses 350 may also have a camera 352 for capturing the user's perspective video image. The camera may be on one side or another of the glasses, or in the center (on the nose bridge or above it). In some embodiments, the camera 352 may be a strip of micro cameras, running over the top edge of the glasses 350. In another embodiment, there may be multiple tiny semi translucent image capturing cells embedded right in the middle of the glasses' display material. In yet other embodiments, the camera may be connected to the human visual system's optical path directly, through a corneal implant, or an intra-ocular implant (FIG. 6A). The general position of the camera is not critical so long as it does not interrupt the line of sight for the user to the patient. The x-ray image 382 may be derived from using either an x-ray source on the sensor garment or an x-ray source inserted into the patient through the garment. The choice of x-ray source and imaging parameters will depend on the health care provider and the type of image the provider desires. In some embodiments, the x-ray image 382 can be combined with the pre-operative CTA scan to form an integrate image modality 384. While x-ray and pre-operative CT scans are mentioned here, the integrated image modality is not limited to these image types. Image information (data) can come from radiography, ultrasound (external and internal), magnetic resonance imaging, nuclear medicine imaging, optical coherence tomography, gamma probe imaging and any other form of imaging technology. The integrated images may be used in various methods as described herein.

In some embodiments, the sensor or detector garment 380 may be large enough to wrap around the chest of a patient (FIG. 3D). The configuration of detectors and x-ray emitters may be varied for individuals of different shapes and sizes, from small children to very large adults. The garment may have fasteners for securing it around the chest. The garment may further have fiducial markers for coordinating the location of the garment and its various elements in a virtual or enhanced reality. The fiducials may be useful in orienting the garment and images produced with it, and then correlating those images with an integrated image modality.

In another embodiment, the sensor garment 360 may have a more rigid frame and have a solid structure like a casing or shell 362 (FIG. 3E). The shell may have lead or other lining to prevent x-rays or other forms of radiation from irradiating anything other than the patient. In this way, the amount of radiation needed to scan the patient is reduced, and the need for other radiation protection gear on HCP staff can be reduced. The sensor garment may have an inner layer 364 having one or more x-ray emitters 366 and x-ray detectors 368. The emitters 366 and detectors 368 may be spaced apart on the inner layer 364 to provide maximum coverage of the patient body. In an alternative embodiment, the shell 362 may be designed to focus on a particular part of the body, such as the heart, lungs or other organs. In still another embodiment, the casing may be custom made, with a cast made of a particular part of a patient, and the casing made from the cast mold to better fit the patient. In some embodiments, the emitter and detector may be one in the same, as in if the sensor used is an ultrasound transducer.

In some embodiments the sensor garment may use alternative sensor or imaging modalities besides x-ray emitters. The sensor garment may have an adaptor for receiving a variety of imaging devices so the emitter and detector may be swappable or interchangeable with another kind of imaging device. These other imaging modalities may include ultrasound imaging or NMR imaging. By swapping out different kinds of imaging sensors in a fixed body design, the framework and position of the imaging and/or sensor elements are known, and mapping data becomes easier to obtain.

In other embodiments, the shell may be made of a somewhat flexible material, or a soft wrap material that can fit to the contours of a patient body or clothes over the body. The sensor garment may include a display screen that allows a user to have the enhanced reality image displayed on the sensor garment. In some embodiments the sensors, detectors and other position markers may contain or use a local position or coordinate system so one or more of the detectors or sensors are able to know the location of one or more of any other element used to establish a three dimensional coordinate system for the sensor garment. In this fashion the sensor garment may be put onto a patient body and the sensor garment may go through a start-up procedure where an element of the 3D sensing system can identify another element and form the 3D map of the body part the sensor garment is wrapped around. As used herein, 3D map refers to the output video image of the system, which includes any visual presentation the system is capable of creating or rendering, from any available data or processing resource the system has access to. Thus reference to any 3D map, 4D map, 2D image, or image that can be presented on a display includes any type of image the system can create, import, render or access, even if the data to be displayed exceeds the physical/electrical limitations of the display device.

In some embodiments the sensor garment may have integrated intelligence. In some embodiments the 3D map can be displayed on an integrated display device. In some embodiments, the sensor garment may be able to connect to a readily available intelligent device, such as a nearby smart phone, tablet or laptop using a wireless communication protocol. The remote display device may have a software application (App) enabling maximum coordination with the sensor garment. In some embodiments the remote display may use internal positioning capability to register itself with the 3D Map of the sensor garment, so if the display moves, the image on screen will adjust accordingly and still provide the user with a usable 3d map, corrected for angle, position data or distance. In some embodiments, the soft body design may have a swappable imaging sensor/detector.

In still other embodiments the sensor garment may possess tissue imaging capabilities by using one of the imaging modalities described herein, or any future tissue imaging modality that becomes commercially available. The sensor garment may have imaging emitters and receivers that are able to automatically aim at each other, that is the imaging elements are able to detect each other and aim at their designated receiving elements to produce an image of the patient tissue.

In another embodiment, there can be a vest garment 380 for a patient to wear during a procedure (FIG. 3F). The vest may have a shielded lining to protect other users and the patient from unnecessary x-ray exposure. The vest garment 380 may have one or more x-ray emitters 384ₐ₋ₙ, and one or more x-ray detectors 382ₐ₋ₙ. The vest garment may have a fastener 386 for holding the garment in place on the patient body. Each x-ray source and detector may have an electrical cable 388ₐ₋ₙ leading out to a computer or other device. In some embodiments, the garment may include a display device incorporated into the garment, so a user can see the enhanced reality image directly on the patient body. The display device can be used like a “window” to see the enhanced reality of the patient interior. An appropriately sized display device may be incorporated to any garment device described herein.

In some embodiments, there may be a wearable sensor device 342 connected to a power source 332 and multiple other devices (FIG. 3G). In some embodiments, there may be one or more x-ray emission devices 344ₐ₋ₙ and display screens 346ₐ₋ₙ. The wearable sensor may have a removable flexible screen 334. The wearable 342 may have multiple built in detectors 338ₐ₋ₙ, and multiple built in x-ray sources 340ₐ₋ₙ. The wearable 342 may also have a fastener 336. A cross section view is also shown.

In another embodiment, the system 300 may include a big picture display 302 connected to a computer system 306 (FIG. 3H). The computer system 306 is in electronic communication with a fiducial marker F used for an anatomical tracker, a tracked tool 310, a wearable tracker 314 and a wearable reusable device 308. The system can include one or more electromagnetic sensor(s) 304, and one or more cameras which may be incorporated into the electromagnetic sensor 304, or may be separate. The wearable reusable device 308 may be a display (mono or stereoscopic), made of flexible fabric like material that drapes on the patient to take the body's natural shape. The flexible material may be a polymer, or weaved fabric or blend. The wearable reusable device 308 may also include shape sensing elements that are used as an input to enhanced reality (ER) image generation sub system, to generate ER images that when displayed on the wearable reusable device's display, look correctly aligned with the underlying and surrounding anatomy, and provide an undistorted, virtual see-through view of the internal clinical context right there on the patient site. A disposable sleeve 316 may be placed over the area of operation containing the wearable tracker 314, tracked sheath 310 and wearable reusable 308.

In an embodiment, the wearable device 308 may contain electronics and sensors capable of replacing or augmenting the function of the computer system 306 and the sensor device 304. The wearable device may contain one or more visualization devices (such as a micro x-ray emitter and x-ray detector or other imaging device, electromagnetic sensor, ultrasound transducer or light diffraction sensor.

In another embodiment, the wearable device 308 may have a passive screen, similar to a projector screen in function, the screen reflects an image presented on it by a projector. The wearable device may have boundaries associated with it that a projector can access, so the projector will only shine the image on the passive screen and not elsewhere.

In some embodiments, the wearable device may be integrated into a chair or bed. The wearable device can be incorporated into a hospital bed, examination table, operating table, gurney, reclining chair, or other furniture with one of the detector or emitter of an imaging modality incorporated into the furniture, and the other in the wearable portion of the device. As one non-limiting example, one can visualize a blood pressure reader attached to an arm chair rest. Similarly a wearable device could be attached to a table or other furniture and be available for a patient to insert a limb or body into the wearable attachment. Alternatively the wearable may have two or more elements and can be used to envelop the patient/body part by overlaying the elements, or having the elements cooperate to form a single wrap or covering. In embodiments where a detector and emitter are used in combination, one may be in the furniture, and one in the wrap elements. Alternatively both may be in the furniture, or both may be in the wrap elements.

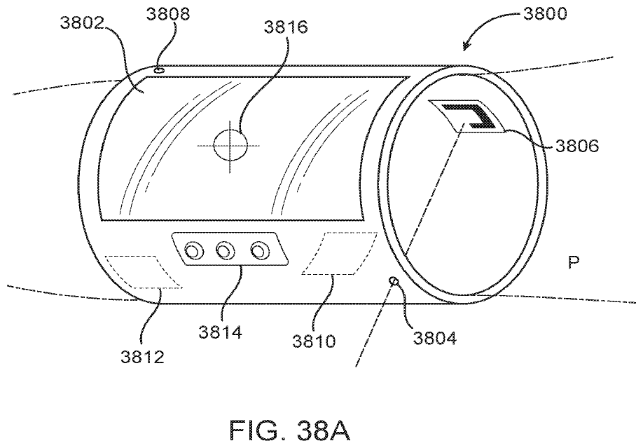

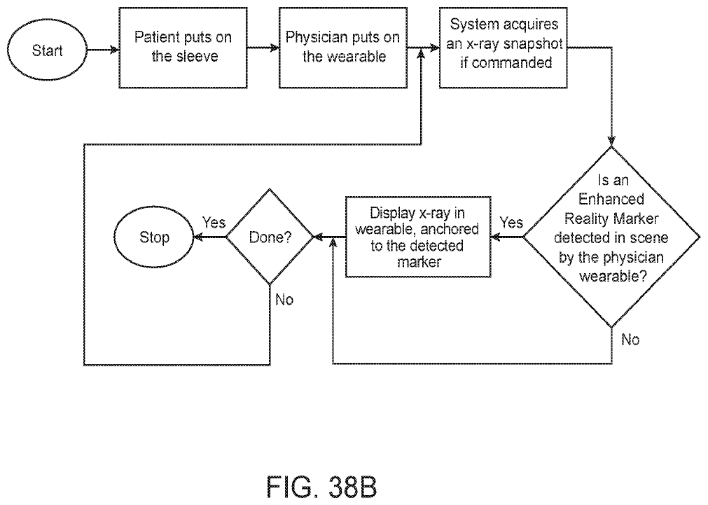

In some embodiments, the wearable sensor device, or wrap around imager device may include one or more enhanced reality markers in fixed positions with respect to the detector(s) for guidance purposes (FIG. 38A). In addition to enhanced reality markers, the device may contain additional elements of the system to allow a self-contained, or nearly self-contained, one piece medical system enabling one or more of the advantages of the entire enhanced reality imaging system. In an embodiment, the device may have a body 3800 that can be wrapped around a patient. The body may have one or more fiducial markers 3806, 3810 or 3812, along with one or more detectors 3808, 3804 for localizing the fiducial markers. The self-contained device my export video image to a user worn device such as glasses or goggle, a portable display device (tablet, laptop, computer screen or smart phone), or have a display device 3802 incorporated into the body 3800. The body may have additional detectors as previously described herein for use in various embodiments of a sensor garment. The self-contained device may have on board electronics 3814 which may include user controls, computer electronics and a power supply (or receptacle for outside power). A computer generated image marker 3816 may be displayed on screen to assist the user in identifying where the area of treatment is, or where the user tool is during a procedure. Other elements of the enhanced reality image may be displayed as desired.

In another embodiment, the self-contained or nearly self-contained enhanced reality imaging garment may be used in a straight forward and simple manner (FIG. 38B). In one aspect, the patient may simply put on the wearable sensor device as directed by the physician or user. The user then puts on a headset or accesses some other display device. The device and display can access each other and set up a virtual reality environment. The device then acquires one or more snap shots using whatever imaging system is onboard the device. The device then integrates the snap shots with the virtual reality to produce the enhanced reality image, and export the enhanced reality image to the display device.

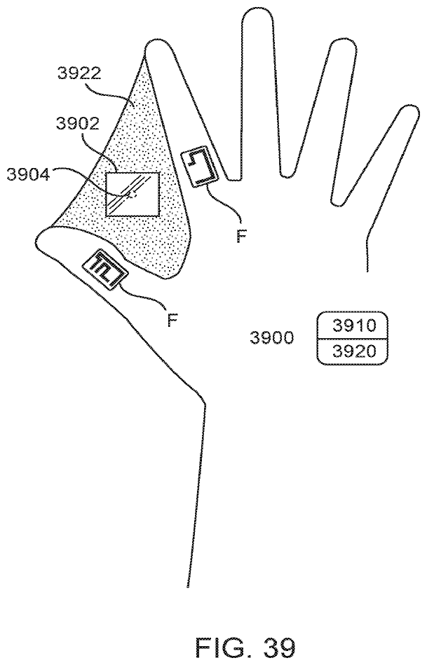

In still another embodiment, the enhanced reality image may be displayed on a glove or hand/wrist attachment (collectively “glove”) to a user's hand (FIG. 39). In this embodiment, a user may wear a glove 3900 having one or more of fiducial markers F, display screen 3902, optional aperture 3904 for tool insertion, optional on board power supply 3910, optional on board electronic intelligence or other elements of the enhanced reality imaging system 3920. The elements on the glove allow the user to visualize the enhanced reality image using the glove during a medical procedure. In one aspect, the image display may be placed on webbing 3922 between the thumb and index finger. In another aspect the glove may be incorporated into the enhanced reality image system so as the glove is moved with respect to the patient, the system can properly track the glove 3900 and move the image and change the view and perspective appropriately. In another aspect, the glove may have a fiducial marker so the glove's position can easily be tracked by the enhanced reality position sensing system. In still another embodiment, the image may be displayed on a third party device having image capabilities (such as a smart watch, fitness tracker or similar device). In still another embodiment, the image may be shown with an aperture in the image, the aperture being useful for the placement of a medical instrument.

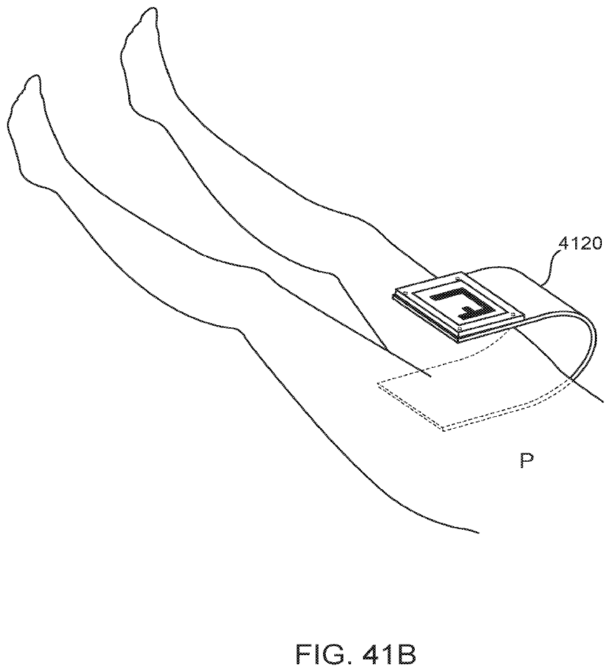

In another embodiment, the fiducial marker and the imaging sensor may be combined into a single unit (FIG. 41A-B). These embodiments may include a series of layers for placement on opposing sides of a patient body. In an aspect, the device may be a U shaped body 4104 for placement on opposing sides of a patient P, such as the torso, limb or extremity. In an aspect, one side of the U shaped device contains the imager (such as an x-ray source material, ultrasound transducer or other imager) 4106. It may be an actual x-ray source seed encased in a material with a window, or it may be an electronically activated material that produces x-rays in response to electrical current. The other side of the U shaped device has a skin tag 4102, imager frame 4108, detector 4110 and enhanced reality marker 4112. The elements are presented in an exploded view in FIG. 41A, and fully assembled 4120 in a possible use view in FIG. 41B. In another embodiment, the entire assembly 4120 may be handheld, with a handle for the user to position it over a body part.

In still another embodiment, the imager 4106 may be positioned on the same side as a detector 4110. In this embodiment, the detector may detect reflected imaging radiation or mechanical energy from the imager. In still another embodiment, there may be multiple modes of imagers and detectors on each side, such as an x-ray source on each side with detectors on each side, and each able to discern direct radiation as well as reflected radiation. In another aspect the imager may be an ultrasound transducer that both transmits and receives ultrasound energy. Other imagers are possible and the unit may have a mix of imagers and detectors in any relation to each other on one side or both sides of the U shaped unit.

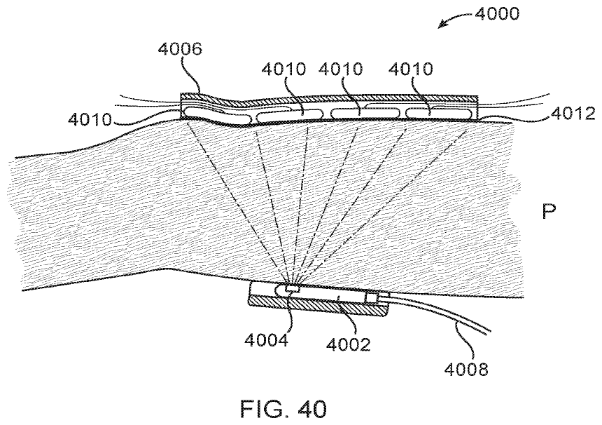

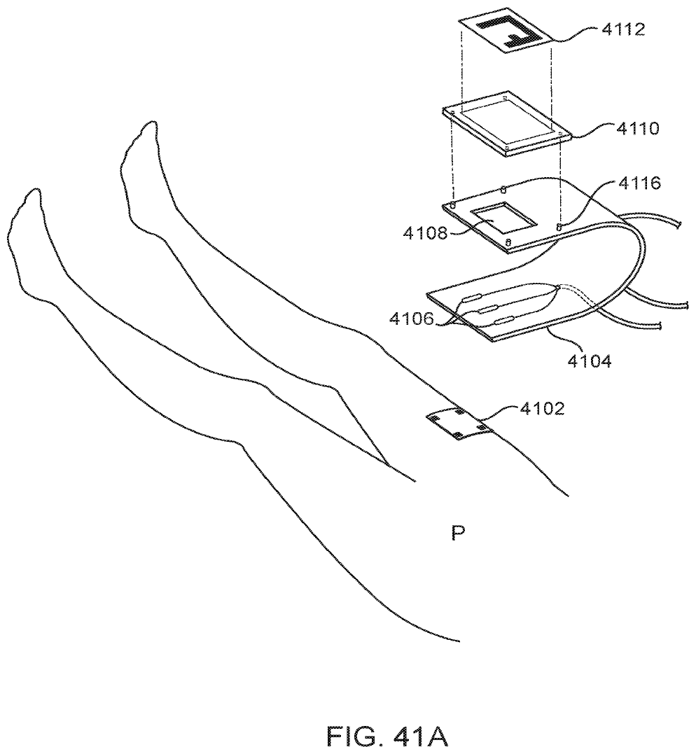

In still another embodiment, there may be one or more detectors 4010 layered between a sheath side 4012 facing a patient body, and a radiation shield backing 4006 opposite the sheath (FIG. 40). One or more micro x-ray device(s) 4002 with a radioactive seed or electrically powered x-ray generator 4004 may be placed on a patient, and project x-rays toward the detectors, so an x-ray image may be taken of a particular body area with minimal radiation leakage or exposure to other persons. In an embodiment, there may be a series of film or electronic detectors 4010, that can form an x-ray image after exposure to x-rays from an x-ray source, such as a micro x-ray source or electronically generated x-ray source. The x-ray detectors can relay the image to a processor and the processor can correlated the multiple images into an enhanced reality image for use in a medical procedure. The radiation shield may be used on any form of sensor garment 4000 or fiducial marker disclosed herein. In an aspect, the radiation shielding may be a draping material over hanging the assembly to reduce the risk of any radiation leaks out of the region of interest.

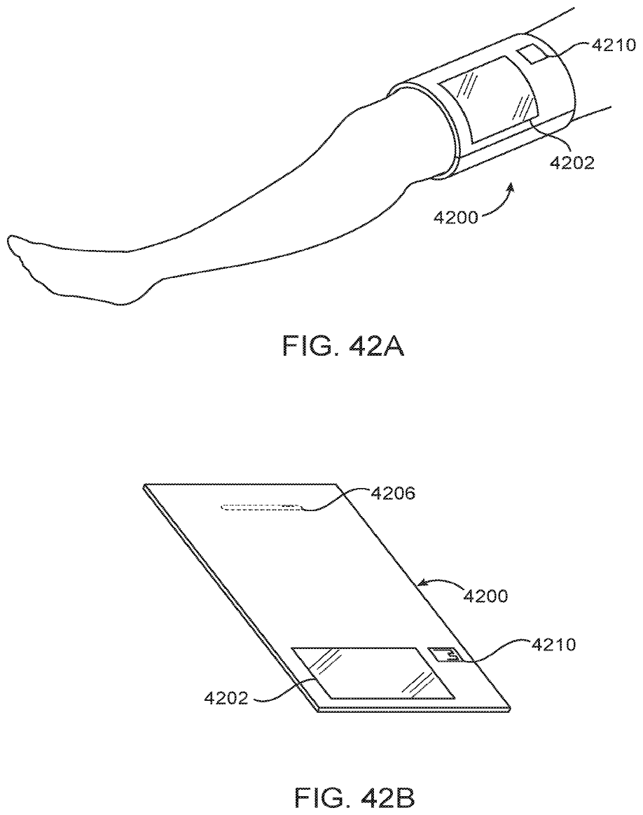

In another embodiment, a micro x-ray source 4206 may be embedded in a sensor garment 4200 with a detector panel 4202 (FIG. 42A-B). The sensor garment may be any kind described herein. The sensor garment 4200 can be wrapped around the patient body, or a limb such as a leg, shown as a non-limiting example. The sensor garment 4200 has an enhanced reality marker 4210 and a detector panel 4202. The position and distance between the enhanced reality marker 4210 and the detector panel 4202 are known for each garment 4200. Also known is the relative size of each and the relative placement of each. The sensor garment 4200 can also be size for patient body, limb or body parts of various sizes and shapes, so when the appropriate garment is placed properly on a patient, the x-ray source is placed in the enhanced reality workspace, and allows for accurate placement of all elements when producing the enhanced reality image. The garment may have an incorporated display, or the user may use a third party display device, goggles, glasses or other display device. In one aspect, the sensor garment is shown wrapped around a leg (FIG. 42A) and also displayed flat with the components revealed (FIG. 42B).

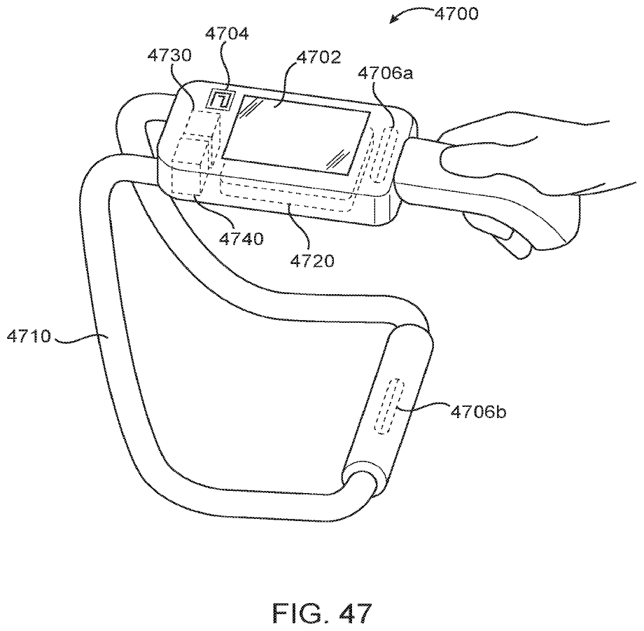

In still another embodiment, there may be a hand held scanner 4700 able to capture image information from an imaging subject's interaction with either a directly transmitted radiation between the source 4706b and detector 4720, or indirectly reflected radiation between the source 4706a information on the detector 4720 (FIG. 47). The hand held scanner has a handle, and an optional attachment 4710 for holding one or more additional imagers like 4706b for use with direct transmission detection imaging. The hand held scanner 4700 may have a fiducial marker 4704, computer electronics 4730 and a power supply 4740. The on board computer electronics may also provide image date to a display 4702.

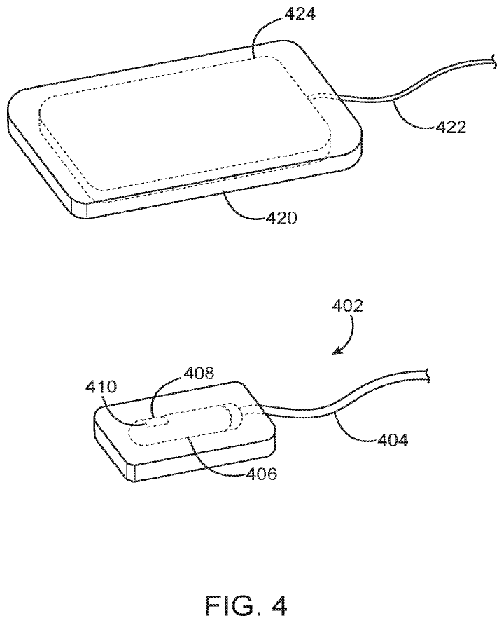

Various devices may be used to produce an x-ray image. In an embodiment, there may be a micro x-ray source 402 having a radiation source 408 contained within a container 406 (FIG. 4). The x-ray source 408 may be a radioactive seed (small mass of radioactive material) or an electronic device able to emit x-rays when energized. The radioactive material or strip is housed within a container 406 to ensure radiation is emitted only in the intended direction, and stray radiation does not irradiate surrounding tissue or people. The container 406 may have a window 410 that can be opened and closed on demand. In one aspect, where the x-ray source is an electronic device that produces x-rays when energized, the window may be a permanent opening in the housing 406, since the x-ray emissions can be controlled electronically, and there is no need to shield the source when it is not energized. In some embodiments, a closable window may be useful to ensure the patient is not accidently exposed to radiation in the event of an unintended energization of the x-ray emitting electronic. The x-ray producing material and housing may be connected to the control unit or intermediate unit via a wire 404, or connected wirelessly.

Images may be produced or captures on an x-ray film 424. The x-ray film may be a traditional film, or a reusable electronic sensor able to capture x-ray images. The film 424 may be contained within a housing 420 and connected to the control unit or intermediate unit via a cable 422, or wirelessly.

In some embodiments, there may be a sensed guidewire 2610 having a SDD 2614 near the distal tip 2612. The sensed guidewire may have electronic leads 2618 connecting the SDD 2614 to a computer, Fiducial Marker or other electronic component. The guidewire 2610 may have a wire braided exterior 2616 similar to other minimally invasive devices, to promote axial flexibility while still providing pushability. The distal tip 2612 can be atraumatic so as to reduce the likelihood of injury to a patient during use. The SDD 2614 may be passive, active or pingable. The SDD can be detected by an electromagnetic field sensor so the tip can be detected in the electromagnetic scan field.

In some embodiments, the guidewire may be dimensionally closer to a small catheter than an actual guidewire. The guidewire may have more than one SDD on it.

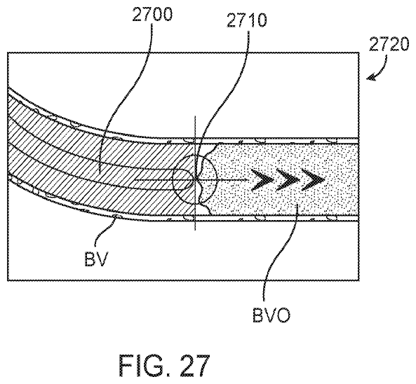

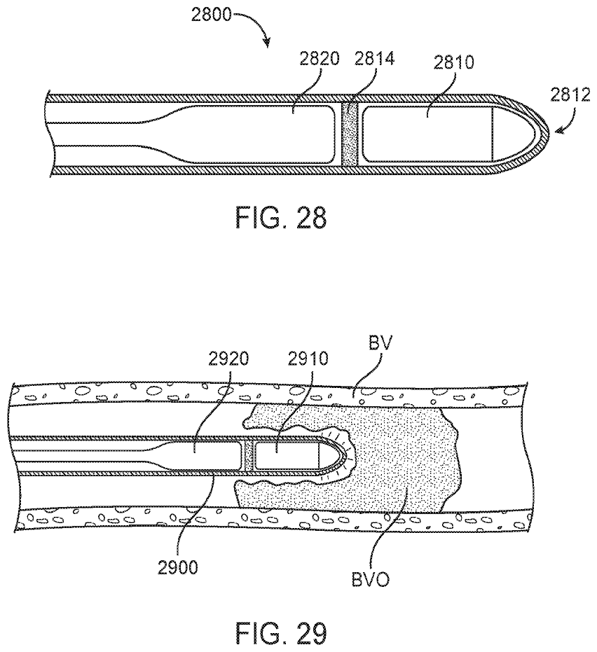

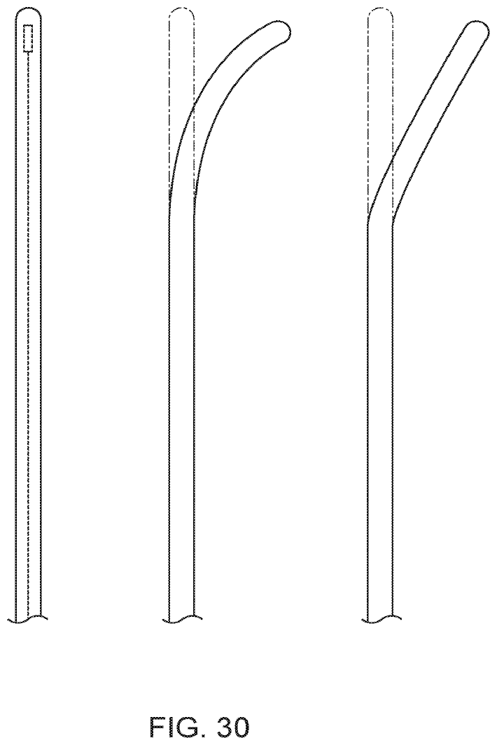

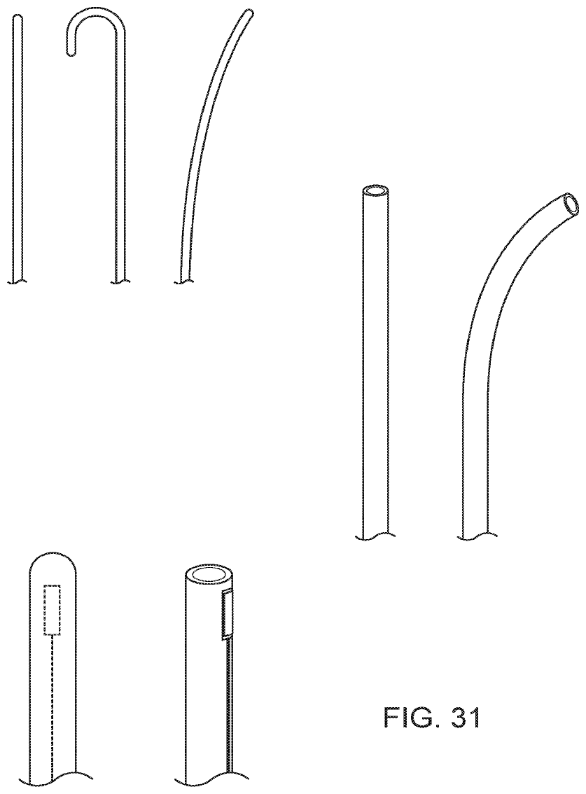

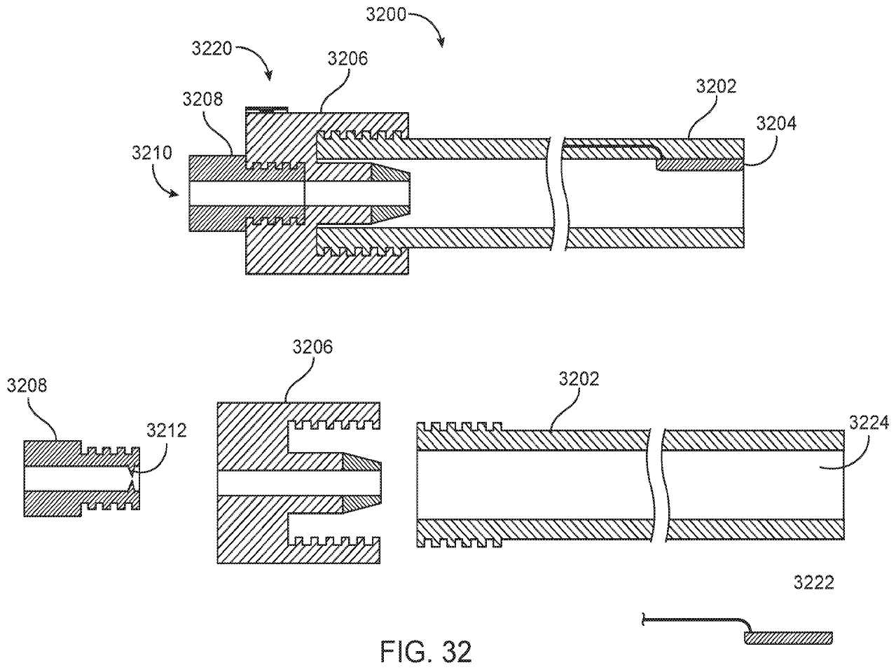

In an embodiment, the guidewire may be tracked within a blood vessel BV and advanced toward a blood vessel occlusion BVO. The guidewire can be advanced through the occlusion to gain the other side. The procedure may be imaged and displayed 2720 on a device or headset/glasses so the physician sees the volume of space the occlusion is in without having to open the patient up (surgery) (FIG. 27). In one aspect, a minimally invasive catheter 2800 may have a SDD 2820 positioned proximal to a heating element 2810. The device can have an atraumatic tip 2812. The SDD 2820 and the heating element 2810 may be separated by a thermal insulation barrier 2814. In another aspect, the catheter with heating element 2900 may be deployed into a blood vessel BV with an occlusion BVO. The heating element 2910 can be used to melt or burn through the occlusion BVO. The catheter 2900 has a SDD 2920 so that the catheter may be tracked by an electromagnetic sensor when the catheter tip is within an electromagnetic field produced by the sensor. The guidewire or catheter with a SSD may be flexible and/or steerable as are other devices well known in the art (FIG. 30). In various embodiments, the SDD may be incorporated in a large number of catheters or guidewires. In some embodiments, the SDD may be embedded into the distal end of the guidewire or catheter. In other embodiments, it may be incorporated into the exterior surface (FIG. 31).

In still other embodiments of catheters and guidewires, there may be a guide catheter 3202 with a SDD 3204 at the distal end, and another SDD 3220 at the proximal end. The two SDDs 3204, 3220 can be used to track the position of the distal tip and proximal end of the guide catheter. In an aspect, there may be a guidewire locking mechanism 3208 that can attach to the proximal end of the guide catheter 3202 via an adaptor 3206. The guidewire locking mechanism 3208 may have a physical or magnetic aperture 3212 for engaging a guidewire and preventing it from axial motion within the guide catheter 3202. In another aspect, a probe sensor 3222 may be attached to the distal end of the guide catheter, the probe sensor designed to read data on a guidewire or other tool passed through the central lumen of the guide catheter.

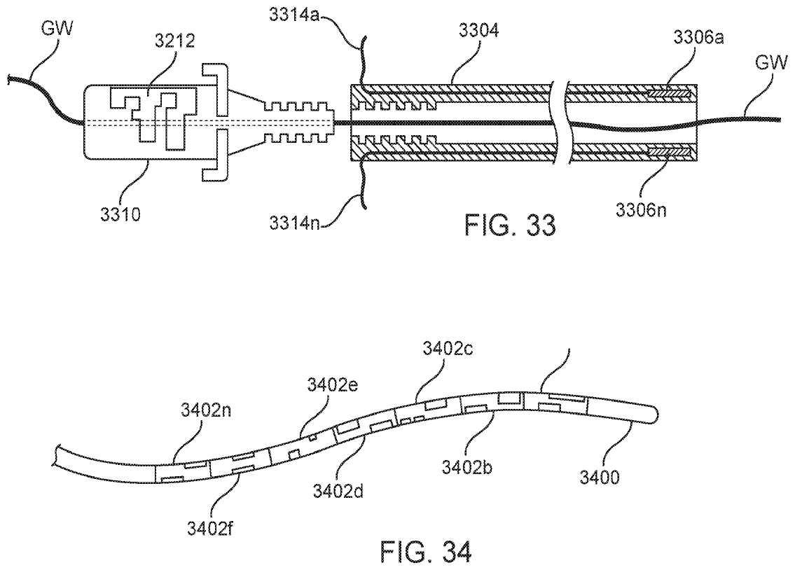

In another embodiment, there may be a guidewire locking device 3310 with direct attachment to a guide catheter 3304 (FIG. 33). The guide catheter 3304 may have one or more sensor probes 3306a, 3306n at a known position near the distal tip of the guide catheter. The guidewire locking mechanism 3310 may have a SDD or visual print fiducial 3312. In another embodiment, there may be a guidewire 3400 having one or more SDD or fiducial markers in the form of a magnetic, optical, thermal or electric feature that can be read by the sensor probe 3306a, 3306n. In an embodiment, the guidewire may be passed through the central lumen of the guide catheter. The length of both the guidewire and guide catheter are known, and by locking the position of the guidewire relative to the guide catheter in the axial direction, an electromagnetic sensor can determine how far the guidewire extends past the distal tip of the guide catheter with great accuracy. The guidewire may have one or more fiducial markers or SDD elements near the distal tip. These may be read by the guide catheter distal sensor probes, and feed back to the system the information read. The information may include physical information of the guidewire such as length, stiffness, diameter and relative distance of each marker from the distal end of the wire. In this manner, the system can accurately determine the distance the guidewire protrudes from the guide catheter regardless of any bending, kinking, twisting, or binding the guidewire may experience inside the guide catheter lumen.

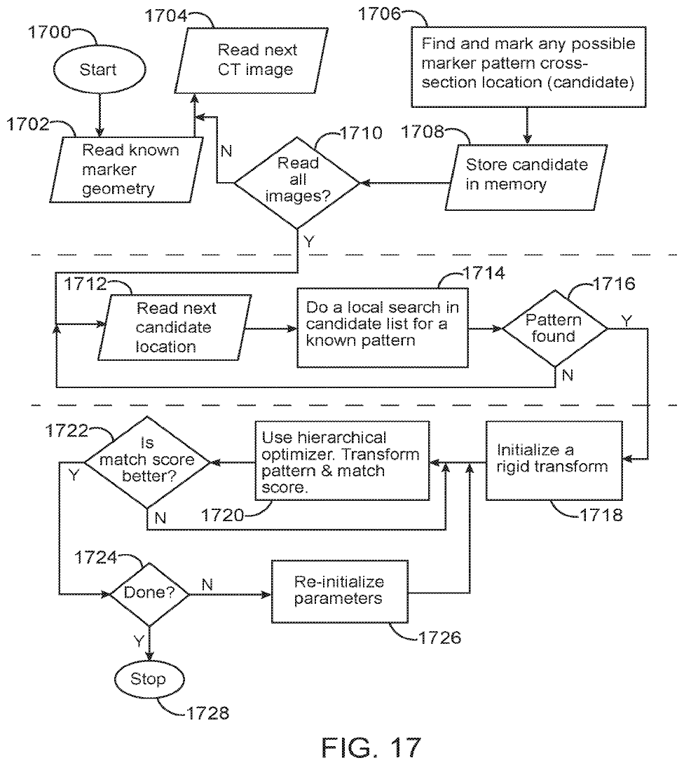

In some embodiments, there may be a tracked guidewire for PAD (Peripheral Arterial Disease) usage (FIG. 17B). In one aspect, the guidewire may have a 0.35 mm diameter at the distal end, with a 0.3 mm core and 0.05 mm cladding wound around the core. The distal end of the wire may have a sensor having 5 or more degrees of electromagnetic freedom. The tip containing the sensor may be rigid or reinforced to protect the sensor. The sensor allows the tip of the guidewire to be seen by non-x-ray means as the wire is used to cross a plaque lesion, or other area of interest in the body. The electromagnetic degrees of freedom allow the wire to be tracked using the system described herein and the wire tip position to be displayed virtually in a 3D model of the surgical sight projected onto the user display.

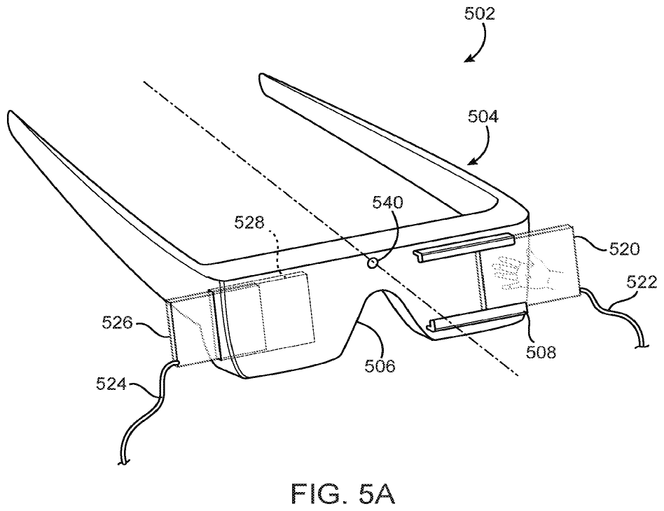

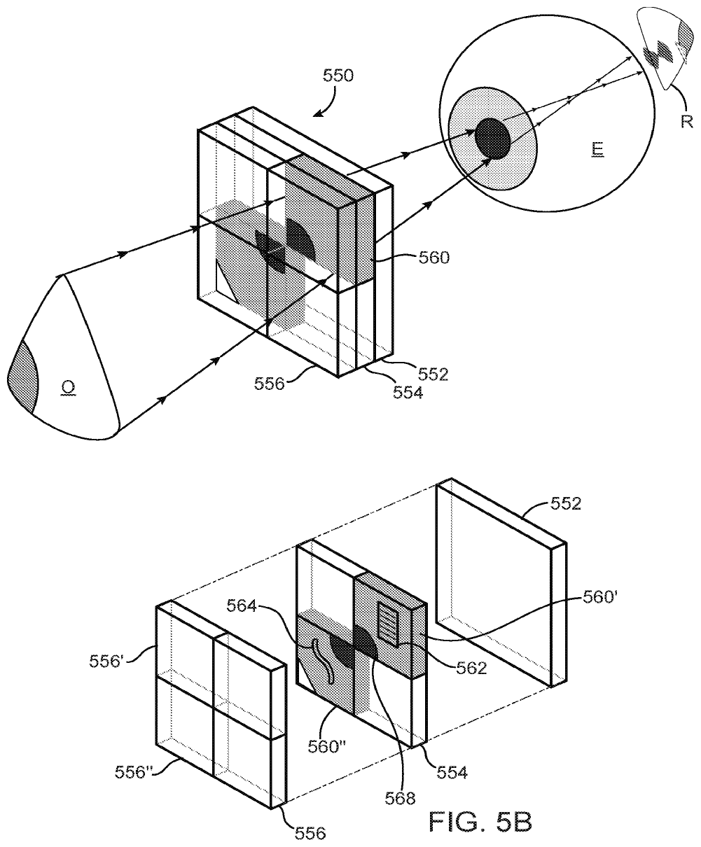

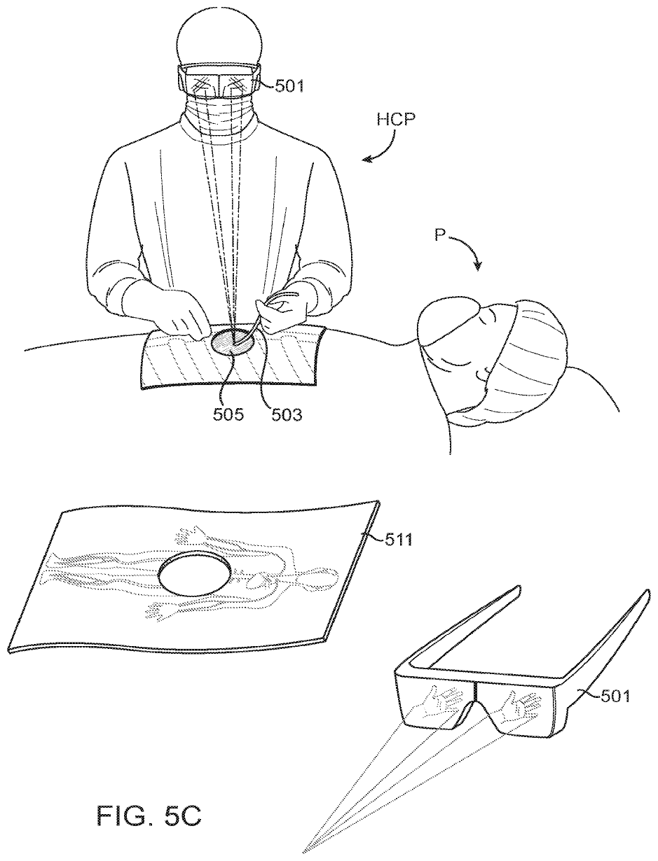

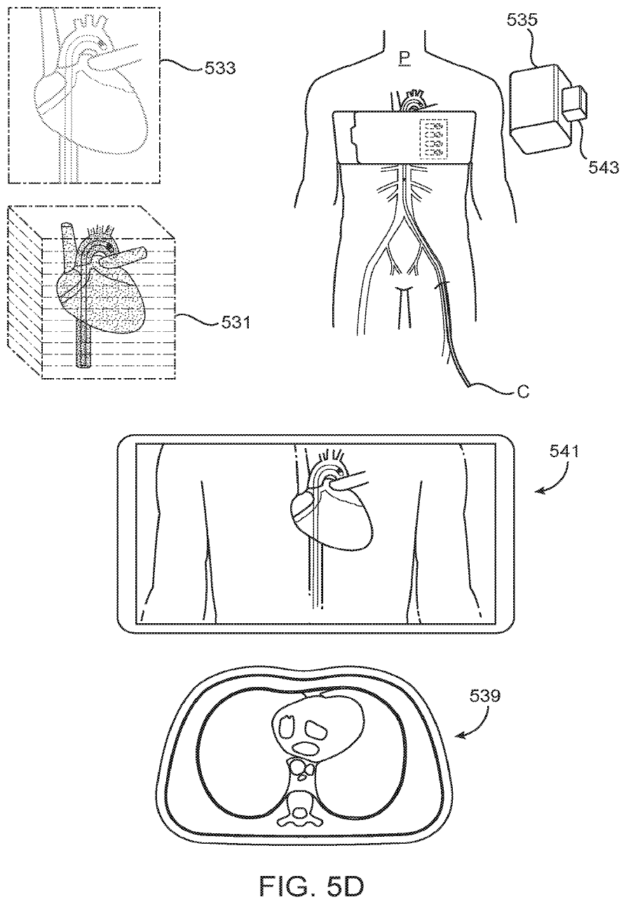

In some embodiments, glasses or goggles 502 may be used to visualize the integrated images (FIG. 5A). The goggles 502 may be any of a variety of currently available “virtual reality” (VR) type eyewear. In some embodiments, specially designed eyewear may be used having a frame 504 and a front plate 506. The front plate 506 may be transparent, or it may be a one or more types of computer display material (OLED, LED, LCD). The glasses may have a forward-facing camera 540 for capturing images directly in front of the person wearing the glasses. In some embodiments, the glasses 502 may have an external mount 508 for holding an insert 520. The insert 520 can be a small computer image display, flexible film display, flexible transparent display or similar material. The insert may have a focusing mechanism so the human eye can focus on it and see the images clearly. The image generated may have an enhanced reality image with compensation pre-built into the insert and/or image generator to trick the HCP's brain into believing the virtual objects presented as part of the enhanced reality are indistinguishable from real objects in depth, shape, texture, size or photorealism. The image and connected via hardwire 522 to a control unit or intermediate unit. In an aspect, the glasses may have one or more internal slot(s) 528 in the front plate 506. The internal slot may receive a small computer image display 526, which may be hard wire 524 connected to an external source for images and/or power. A bisecting plane 510 is illustrated merely to show the left and right half as alternate embodiments. The goggles 502 may have self-contained screens for projecting computer images, similar to a wearable heads up display (HUD) design in other commercial products. The individual lenses of the front plate may be polarized to provide three-dimensional viewing (with one side being polarized at an orthogonal angle to the other side).

The goggles 502 may use a hybrid lens and image display system having two, three, or more distinct components (FIG. 5B). In an embodiment, the hybrid lens may have an enhanced reality layer 554 (ERL) sandwiched between an enhanced reality transformer layer 552 (ERTL) and a vision correction layer 556 (VCL). The vision correction layer 556 can be customized for each individual user. The VCL provides normal vision correction for the user in the same way that prescription glasses do. If the user does not need vision correction, then this layer may be a non-corrective structural layer of glass or plastic material similar to that used for vision correction glasses. The VCL can provide enhanced structural integrity to the goggles. The ERL 554 may be made of organic LED (OLED) material, as that material is semi-transparent and allows light to pass through it. The ERL can also be made of specialized light guide elements that allow display of enhanced reality information up close to the user's eyes. The ERL can be formed to be part way through the field of vision of the user, or all the way, so it has the same area as the VCL. The ERL can receive display images from a control unit, cloud source or other compatible image source. The ERL receives image data and displays it in statically or dynamially alternating patterns so the field of view for the user is not 100% obstructed by virtual image data. The alternating patterns can be synched to optimal presentation modes for still images, text 562 and video streaming 564 (collectively display data or video data). The ERTL has programmable cells that can be made opaque on demand. The cells can also render video data in pieces (some data in some cells 560′, some data in other cells 560″, to form a whole perceived image for the user. Any number of cells per layer, and cell arrangement may be used. While the image data is displayed for the user, the user can still see an object O in the normal field of view, through the goggle lens 550. Images of the object O, and virtual objects 568, pass through the eye E and are displayed normally on the retina R of the user. Virtual objects 568 include text 562, video images 564, and any other image data displayed.

The visual correction layer 556 may have cells 556′, 556″ corresponding to the ERL cells 560′, 560″ so the VCL cells can be “on” or “off” opposite the underlying ERL cells. The third layer ERTL also has cells that can be activated if the super-positioned ERL cell is “on” or “see-thru”. In another embodiment, the goggles may have a component that estimates the direction and depth of focus of the HCP's eyes to allow changing the rendering and presentation of the virtual information in a way that naturally blends with reality. In one non-limiting example, when the HCP's vision is focused on the patient's body skin, only the virtual objects that should be contextually in that area and at that depth of focus will appear. The rest of the virtual information may blend in with the background (blurred or dimmed or smoked away)).

In another embodiment, the HCP may have a wearable display device 501 and look down on a surgical site 505 having a flexible display 511 placed around the surgical site (FIG. 5C). The flexible display 511 may be in electronic communication with the control unit or backend system, and have visual information displayed on it to show the HCP where tools and organs of interest are. The flexible display 511 can be placed on the patient P during surgery. A surgeon HCP may insert or manipulate a tool 503 while operating on a patient and be able to see the displayed image of the surgical site on the flexible display 511. The image data that can be shown on the flexible display 511 or in the wearable display 501 may vary (FIG. 5D). In some embodiments, the image may be a virtual image of the organ of interest 533. In other embodiments, it may be a pre-scan image, such as a CTA 3D image of the organ of interest 531. In other embodiments, it may be the volume of tissue being scanned by the sensor garment 539. In still other embodiments it may be the enhanced reality image 541 produced from the systems and methods described herein. The images shown on the flexible display or wearable display may be archived information or data generated from a surgical procedure. In an embodiment, there may be a catheter C inserted into patient P. The catheter C may be advanced into a region of the body where it can be detected by a sensor garment 543. The image data is handled by a control unit 535, with sensing of the catheter C handled in part by the electromagnetic sensor 537.

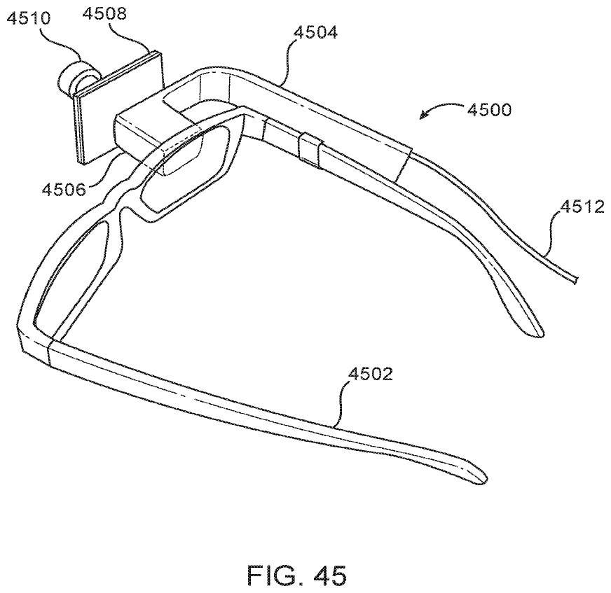

In another embodiment, a normal pair of glasses 4502 may have a combination display 4506 and camera 4510 in front of one or both lenses (FIG. 45). The combination display 4506 and camera 4510 provides the display device for the user so the user can see the real world through the display. The camera shows what is in front of the user while the display shows what the camera “sees” combined with the enhanced reality image of the imaging system. The enhanced reality display attachment 4500 may be connected with a clip 4504 or other fastener to the glasses 4502. The attachment may be self-powered and communicate via WiFi or other wireless communication protocol, or it may have a power line 4512 that may include a data line. In one embodiment, an adhesive backing 4508 is used to put the camera 4510 and display 4506 together, however an integrated device would include the camera and display in a single, ergonomic body without the need for an adhesive backing.

In another embodiment, the attachment between the glasses and the display and camera assembly may be smart enough to know the relative position and orientation of the assembly with respect to the glasses. This intelligence can feed into the processing computer to help auto generate an enhanced reality image that can overlay on the real world objects. This setup may allow for a ‘snap on’ enhanced reality component that can be used with regular glasses that physicians wear anyways, to automatically use their ‘powered’ lenses.

In another embodiment, a wearable contact lens may contain either a miniature screen on it for providing enhanced reality viewing to a user (FIG. 6). In some embodiments, a wearable corneal display 600 may be controlled remotely via an image source. The image source can display the integrated imaging information on the wearable corneal display. In one aspect, the corneal display may have augmented display pixels and see through pixels. The see through and augmented display pixels 612 may be arranged in various combinations so the user can get the integrated image projection and still have some areas of normal vision where the user can see the area in front of them. The pixels may be alternating augmented and see through (like a chess board) 606, arranged in concentric circles of alternating type 608, or have sections of the wearable corneal display established for augmented image display, such as having a dedicated portion of the corneal display set up for receiving or showing the augmented image. In some embodiments, a tiny power supply 604 and/or a communication chip and antenna 602 may be attached directly to the wearable corneal device. In various embodiment, the image of a virtual object (Vₒ) has properties similar to a real object. As the virtual object gets closer than the real object enhances, the eyes struggle to keep both in focus and vergence. Depending on the amount of mismatch between the two representations, this can present a severe accommodation challenge to the user when using existing AR devices.

In some embodiments, an enhanced reality display 610 may take the form of a visor or face shield (FIG. 6B-6C). The enhanced reality display 610 may have a region that can be a polarizable converging lens (for example power +6 diopter) 616, and a second region that is a polarizable see through display 618. A side view of the enhanced reality display 610 shows an OLED (organic light emitting diode) display 612 or 614 positioned above the eyes of the wearer and angled toward the polarizable see through display. The OLED image may be projected by a pair of enhanced reality light engines 612, 614 and can reflect off the polarizable see through display 618 and through the region that is the polarizable converging lens 616. In this embodiment, two light engines are used to provide separate images for the left and right eye. Separate images for each eye can be a way to provide a three dimensional image the user can visually comprehend. In some embodiments, it can also allow the projection of different images at different frame rates so the user can “see” informa0tion from the light engines while still seeing the actual environment through the polarizable see through lens 618. The light engines 612, 614 may be positioned in the enhanced reality display head set 610, or placed remotely such as in a computer. In an embodiment where the light engines reside in a computer or other device with sufficient computational power, the computer may have a single light engine for producing dual images. In some embodiments, the converging lens portion and the see-through display are separate as shown. In other embodiments, they may be layered into a single physical layer. In another embodiment, there may be a third layer having an at least partially transparent to completely transparent OLED or (D) LCD display, backed with an electronically tunable focal length lens matrix. The third layer may be referred to as enhanced reality display layer.

In another embodiment of the display device, the output of the light engine(s) 612, 614 may be positioned to project an image through a variable focus lens 622, and to a first reflector 624 and to a second at least partially transparent second reflector 626 and then into an eye E. The lens may have the ability to change focus in demand. This can be achieved by using any technique known in the art for variable focus, which can be achieved in various non-limiting examples such as electronic image control, physical combination of lenses, electro-chemical controlled lenses, etcetera. In an embodiment, the image projection can be used to change the depth of rendering of a virtual object by using the lens of variable focus. By adjusting the focal depth of the virtual object, it is possible to match the ‘vergence’ point with the focus point. The virtual plane 630 provides the depth for the virtual object.

In another embodiment of the display device, there may be a wearable head set 630 with a face shield 636 or mask having a built in light engine 612 or receiving a video input from an external source (FIG. 6E). The face shield may perform a similar function as a polarizable see through display. The face shield may have a pair of light deflection units which are also at least partially transparent. The light deflection units 632, 634 can receive enhanced reality image field from the light engine(s) or another source and display them. In another embodiment, the light deflection units may be large, panel displays 638, 639 (FIG. 6F). In yet another embodiment, 638 and 639 may be part of an ERHM display, made of a transient nebulous (cloudy) material (FIG. 6F, 638) that lets normal light through but partially blocks (and thus displays) a special kind of light projected from goggles 180, or another projection medium.

In yet another embodiment, there can be a system for auto-focal plane detection for use in an enhanced reality image system (FIG. 6G). In an embodiment, the user may wear glasses or goggles 640 having a pair of eye camera 642ₐ, 642_{b} can be used to capture video images. The system can compute the line of sight LOS₁, and determine the distance of the first object line of sight LOS₁, from the average distance of each eye D₁. Then the system can set the optimal depth of the field zone at D₁. The system can then render an artificial reality image 644 to be viewed as if it were at D₁. The process can be repeated for the other eye using line of sight 2 LOS₂. The augmented information can be displayed on any of the display devices used with the present system. Once the images have been rendered the operation is complete. In yet another embodiment, the location of enhanced reality focal plane may be set by the HCP, knowing what information they need next, and at what depth. The HCP may use a visual, audio, or tactile gesture on the wearable or another part of the system to manually adjust the depth of focus for enhanced reality display. In some embodiments, there may be multiple virtual objects rendered in the HCP's clinical field of view, and depending on the current depth of focus and vergence setup, the remaining virtual objects may be rendered appropriately out of focus to match the rest of the visual context. In another embodiment, a preferred depth of focus and vergence may be preset, knowing the type of medical procedure, the typical working position, and distance of HCP's eyes from the patient site. This preset can be validated and refined if needed to match the HCP's accommodation and comfort before an intervention begins.

In some embodiments, the system may render partial or complete virtual objects at different depths of focus, to match how human visual system functions. This can be achieved in multiple ways, one embodiment may employ a single set of left and right light engines and display apparatus to display pre-processed, depth vergence and focus corrected images. In yet another embodiment, virtual objects at multiple depth of focus and vergence points may be displayed using a stack of display apparatus described earlier, e.g. a stack of 550 (FIG. 5B) per focal plane.

In some embodiments, additional objects 646, 648 represent differently shaped objects, sitting at different depths and vergence points in the visual scene. These objects 646, 648 demonstrate how the focus and vergence change when the HCP's eyes are gazing at one or the other. The gaze can be sensed directly (watching the HCP's eye movement) or using a prediction engine. The prediction engine may use prior knowledge of what the HCP may likely want to look at in the patient site when performing a known procedure).

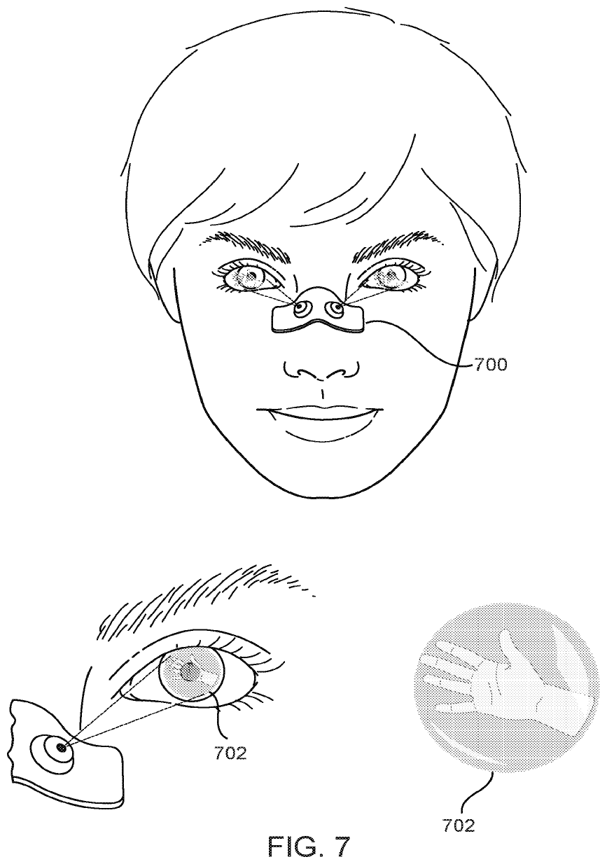

In still another embodiment, the wearable contact lens may act as a screen allowing information to be projected directly onto the contact lens (FIG. 7). In some embodiments, there may be a nose wearable projector 700 able to project an image onto the lens of a person's eye. In an alternative embodiment, the nose wearable projector 700 can project an image onto a corneal display 702 or ordinary contact lens. In some embodiments, the contact lens wearable display may have a focusing optical layer in the assembly to ensure the virtual image may be displayed properly to the human eye. In other embodiments, the wearable 700 may project images on to a screen or the patient body. The wearable may have an aiming sensor to detect when the device is properly aimed at an acceptable screen or skin surface so the image projected may be viewed by the user.

The enhanced reality image may be generated by using a combination of one or more computer driven processes. In some embodiments, various processes for detection of candidate marker locations may be used to establish one or more base positions of the fiducial markers, using one or both of the visual pattern or the SDD positions detected by an electromagnetic field sensor. The term candidate or candidate shape as used herein only for the methods, refers to the shape detected in scanned image data or visual images. The term reference shape means the CAD model geometry of the marker geometry setup.

In some embodiments, there can be a process for marker detection (FIG. 17). This process can be thought of loosely as looking for at least one SDD marker in each image, and disregarding images without a SDD marker. The process starts 1700 when a user initiates the process, and begins reading known marker geometries 1702 from a library. The known marker geometries are predefined by the system and may be one or more coordinates for two dimensional or three dimensional shapes. The shapes may be a single line, or a simple pattern like a square, rectangle or diamond. In some embodiments, the shape may be a complex design with multiple points and lines connecting some or all of the points. The marker geometry can be a computer model (like a computer aided design (CAD) model) that provides ideal position markers for later use. The marker geometry may be a blue print for position markers in establishing correlation with the IPD data. Once the known marker is selected, the process selects and reads a scan image 1704 (CT, MM or other internal anatomy image no matter how generated) and imposes the marker geometry into a general area of the scan image based on prior knowledge of positioning of the marker on the patient. The marker geometry does not need to line up to the same defined origin of the scan image. Scan images often have a point of origin determined by the machine that created the image. While this origin information can be known to the current system, it is not necessary for the current system to rely on the scan image origin, or any other position information provided by the scan image device. So, long as the process accurately tracks the order of the image data and can properly put those images in the same order as they were imaged, the process can operate successfully. The process of imposing the marker geometry 1706 onto the scan image can be used independently from one scan image to the other (the marker geometry can remain the same). The system can impose the geometry marker to the image by correlating features in the scan image that have a similar pattern or position to the marker geometry. The marker geometry and scan image combination are stored in memory and the system continues until all scan images are read. This concludes the detection of candidate marker locations.

In lose terms, it might be thought of as using stars to define a constellation. From Earth, we see a “planar” view of the sky and use that fixed position of the stars (the reference marker geometry) to anchor an image we draw from memory or a different instance of time (the scan image). Each night our relative position in the heavens changes slightly relative to the constellations, yet we still use the geometry of the stars (the geometry marker) to define the constellations, even though they may bend or warp during the seasons. The movement of the earth and the changing perspective of our view can be thought of as different scan images for a patient anatomy. The imposition and perturbation of the marker geometry on the scan image produces a candidate image, with the reference geometry grossly aligned with the scan image. Each candidate image with a coarse such correlation is then stored into memory or cached. The system repeats this process until all images are read and a candidate image has been created for each image. In the next step, the system can search for one or more three-dimensional reference marker pattern(s) in the stack of candidate scan images (the candidate scan image stack represents a 3D volume, but so far, the only match information the system has may be a list of scan images with marker projections visible in the scan image cross sections. These images form the list of candidates scattered individually in each candidate image.) Next the system may ‘build’ a 3D geometry from candidate cross sections that were marked in candidate images. Candidate cross sections or projections that do not ‘fit’ the ideal geometry may be rejected. The position and orientation of the 3D candidate marker geometry may be ‘perturbed’ in ‘intelligent’ steps until the score of match between the instantaneous marker geometry and the reference marker geometry reaches a pre-determined maximum value. At this point, the match can be accepted, resulting in an enhancement of the ‘real’ pattern in the sky with one from memory.

Once the detection of candidate marker locations is complete, the system can build a pattern using known geometry. (This portion of the process can be thought of as the system looking for patterns of multiple SDDs in the images.) The stored candidate images can be read in turn 1712, and a local search can be done in each image to see if there is a list for a known pattern 1714. If a pattern is found 1716, the process may move to the next step. If the pattern is not found, the process repeats on those image candidates with a further refined algorithm. The process may initialize the value of a match score to 0.0 units. Then each subsequent iteration of refinement then improves on the match score, and stops when the current match score reaches a predefined threshold value, or has stopped changing at all. Once a known pattern is found, the process moves to marker pattern refinement.

In marker pattern refinement, the system begins to initialize a rigid transformation 1718. Each candidate image can be processed to optimize parameters and transform a pattern and re-compute the match score 1720. The system may have some intelligence to assist with this process. If the match score can be evaluated 1722 against a threshold value. If the match score is better than the threshold value, the pattern refinement is done 1724 and the process can stop 1728. If the match score is not better than the threshold value, then the marker refinement can be repeated with finer transform adjustments. The parameters can be reinitialized 1726 and the hierarchical optimization parameters transform step can be repeated. This process can loosely be thought of as making all the images stack up into a coherent 3D model. The process may also be repeated continuously as a medical procedure is underway, to improve the marker detection accuracy.

In some embodiments, the process of optimization may use a hierarchical optimizer that performs a gross optimization to roughly determine the position and orientation of each candidate shape (what is detected in an image scan or visual image) in the vicinity of a reference shape (the CAD model geometry). Then the process may do fine optimization starting with the gross optimization data and refine the position and orientation of the detected SDDs using a weighted sum of various errors such as; average angular position, positional correlation over the entire shapes, error fit of the reference SDD over intensity data in the image scan data and projected correlation error at certain landmarks in each image. The process may be repeated to refine the data until the margin of error reaches an acceptable threshold value (measured in distance, angles or other values).

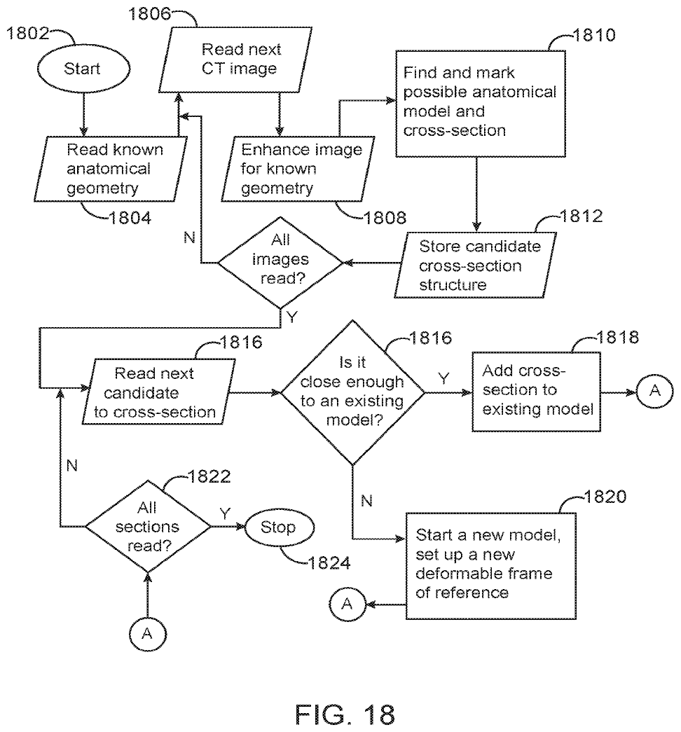

In some embodiments, there can be a process for deformable model extraction (FIG. 18). The process can be initiated 1802 manually or by machine trigger. In this process, the system can read known anatomical geometry 1804 of the interiors of the imaged organs in question. The system then reads the scan images 1806 provided and enhances the scan images with known geometry of imaged organs 1808. The process can then find and mark possible (candidate) anatomical model and cross sections 1810. The candidate cross sections are stored into memory 1812 until all images are read 1814. Any images that were not successfully made into cross section structures are placed into the queue for re-evaluation with an appropriate scan image. Once all images are read, the system reads the next candidate cross section 1816. If the candidate cross section is ‘close enough’ to an existing model, the cross section is accepted and added to the existing model 1818. If the cross section is not close enough to an existing model 1816, the system starts a new model by setting up a new ‘deformable’ frame of reference 1820. Once all sections are read 1822, the process stops 1824. If any section remains unread, it is placed in queue again for reading of the next candidate cross section 1816. The process described may be loosely thought of as two processes, one for extraction of a ‘candidate’ cross section, and another for building of a deformable enhanced reality model set.

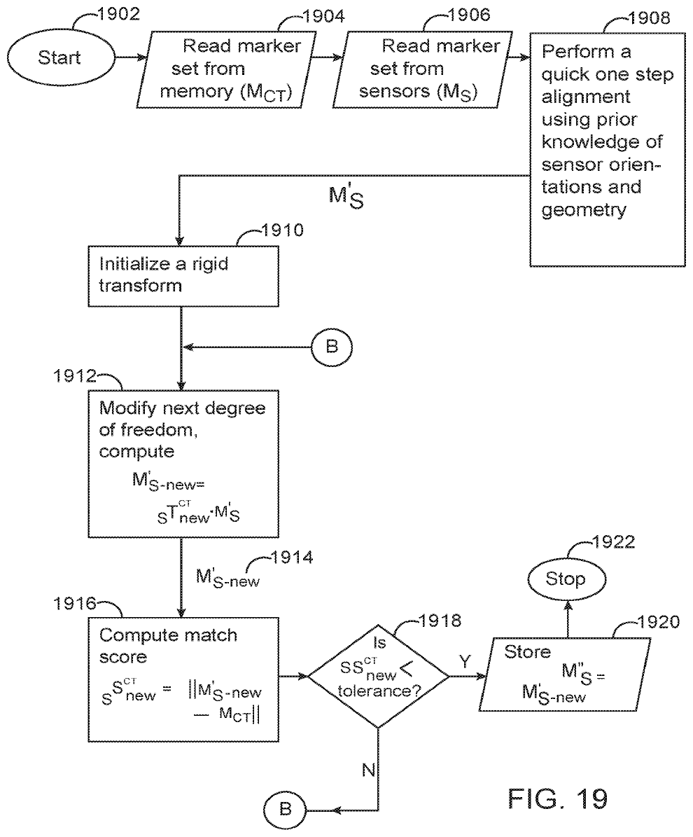

In some embodiments, there can be a pre-operative and intra-operative process for correlation of markers (FIG. 19). This process can be used to correlate pre-operative and scan image data with intra-operative data based on sensed markers during or prior to a procedure. In an embodiment, the system can read a marker set from a memory device (M_{CT}) 1904, read a marker set from sensors (Mₛ) 1906 and then do a quick one step alignment using prior knowledge of sensor orientation and geometry 1908. The aligned data (M′ₛ) can be analyzed using a rigid transformation 1910. Then modify next degree of freedom and compute 1912:

M′_{s-new}(1914)=ₛT^{CT}_{new}×M′s,

Then compute a match score 1916:

ₛS^{CT}_{new}=∥M′_{s-new}−M_{CT}∥

The ₛS^{CT}_{new} value is compared against a threshold tolerance 1918, and if its less than the tolerance, then the value can be recalculated by reprocessing as a post rigid transformation value. If the value is equal to or better than the tolerance limit, the data can be stored 1920:

M″ₛ=M′_{s-new}

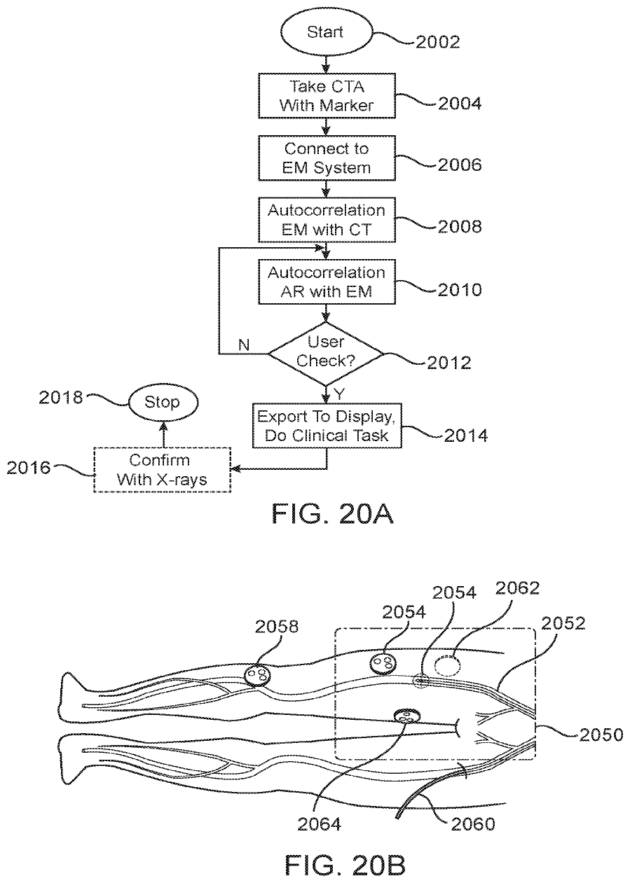

In another embodiment, there can be a method for a mixed reality endo-vascular image guidance (FIG. 20A-20B). The method can take advantage of devices and systems described herein. In one aspect, the method may use image scan data combined with one or more fiducial marker positions 2004. The system can then connect to an electromagnetic sensor system or device 2006. The two image types can be correlated 2008, and combined with an image correlation with a visual image and the electromagnetic image set 2010. A user check 2012 can be used to verify the correlation. The combined image information is output to a display device 2014 while the user performs a medical procedure. The user may confirm the model with an x-ray/fluoroscopy device 2016 if desired. When the medical procedure is finished, the can process end. The various image data for the method can be derived from a visual image captured by a camera, and using the fiducial markers 2058, 2054, 2062 or 2064 as reference points to help correlate the visual picture. The image scan data can come from a previous scan of the patient body before the medical procedure starts. The patient would have the same fiducial markers in as close to the same place as possible (same fiducial marker positions as much as possible for image scan and visual scan and electromagnetic sensor scan). The electromagnetic sensor can detect the SDD elements within the fiducial marker and line up the marker positions on the scan image data. This allows the correlation of the electromagnetic and image data 2006, and the autocorrelation of the visual and electromagnetic data 2010. In addition to the use of fiducial markers, the procedure may correlate position data for a catheter 2060 having a SDD 2056 at the tip of the distal end. The enhanced reality image 2050 provides the user with a view of the patient's inside so the user may feel like he has “x-ray” vision, and can see through the patient body and “see” the blood vessel and tissue volume the user is performing a medical procedure on.

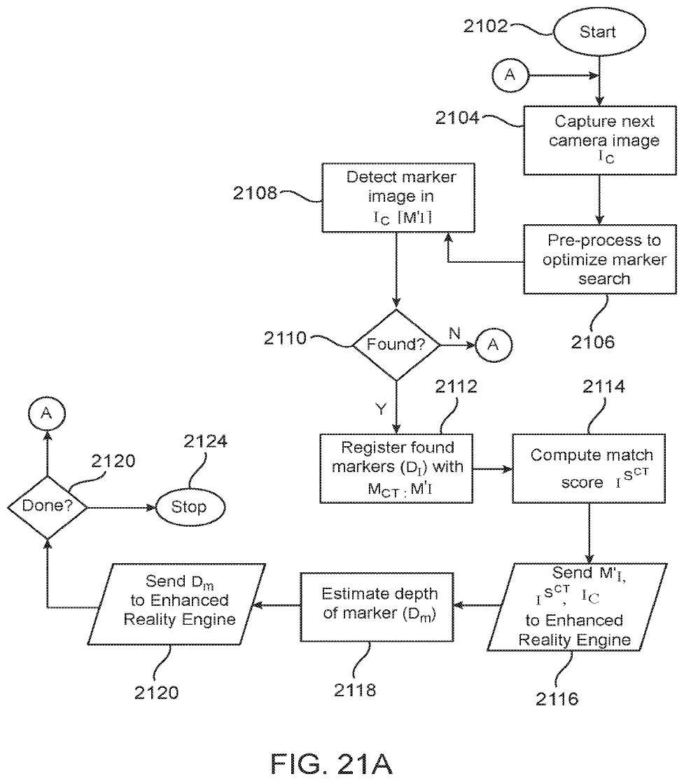

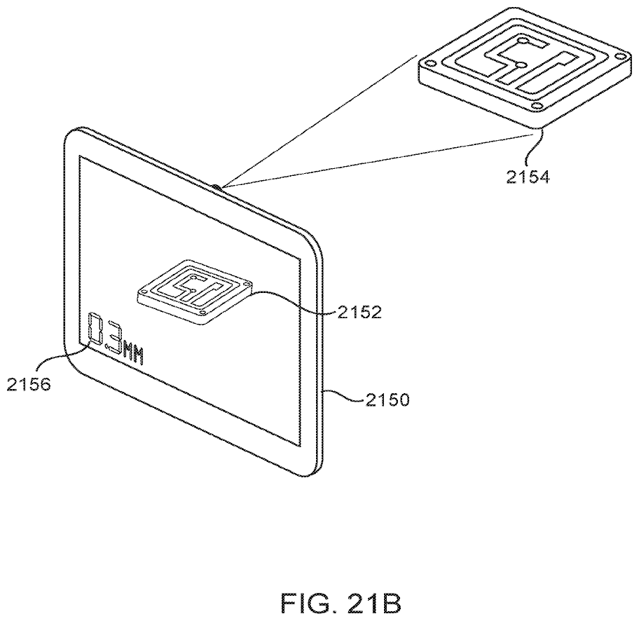

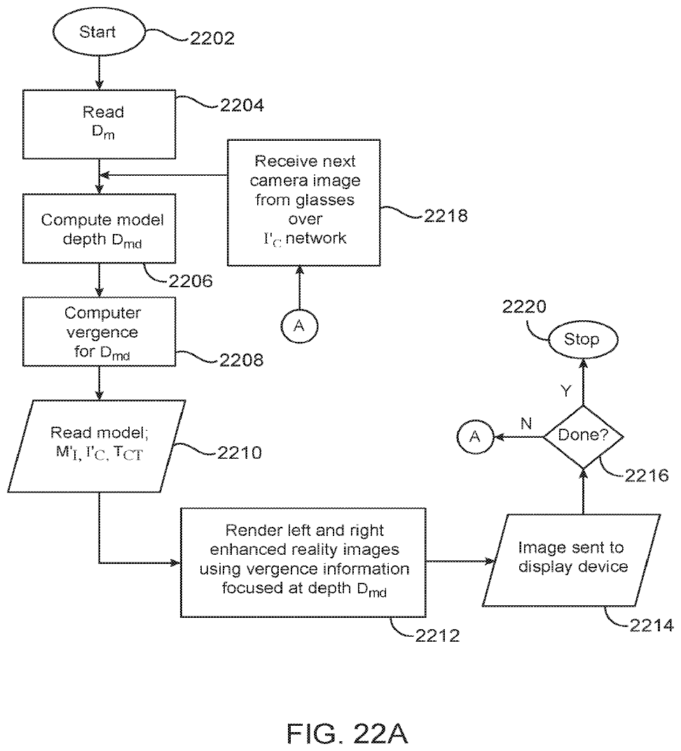

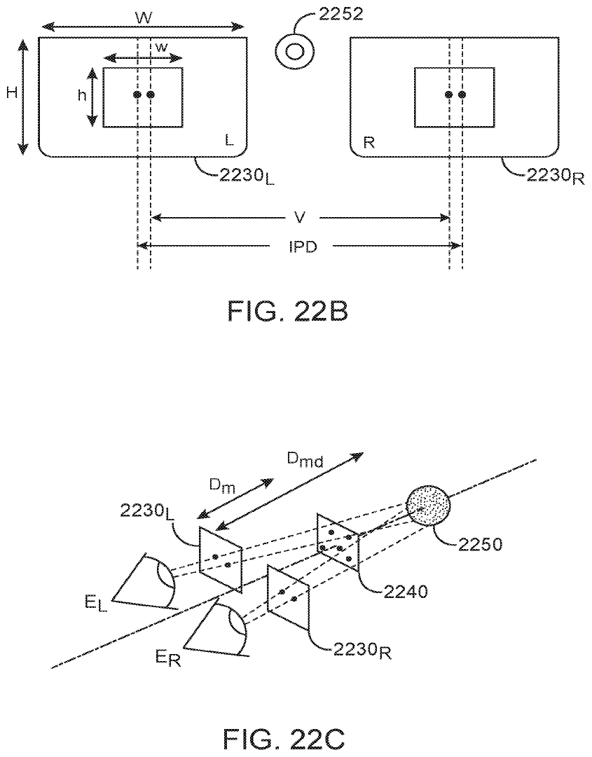

In some embodiments, there can be a camera used to capture images of the patient body during a medical procedure (FIG. 21B) that can be used for camera and image scan registration (FIG. 21A). The camera may be mounted on a user's body, providing a visual scan with the same view as the user, or the camera may be mounted somewhere in the procedural space. Multiple cameras may be used. The process captures camera image data (Iᵣ) 2104 and pre-process the image to prepare it for marker search 2106. The system attempts to identify markers in the image Ic [Mc] 2108. The system determines if a marker is found 2110. If the markers are not found, the image is rejected and a new image is captured 2104. If the markers are found (M_{I}), they are registered with M_{CT} (result: M′_{I}) 2112. Once the markers are registered, the system computes a match score _{I}S^{CT} 2114. The system sends M′_{I}, _{I}S^{CT}, I_{c} to the enhanced reality engine 2116 (See FIG. 22). The system can then estimate the depth of the markers (Dₘ) 2118 and send the Dₘ to the enhanced reality engine 2120. This process may be considered done 2122 at this point if the score _{I}S^{CT} is ‘close enough’ to a pre-defined threshold value. Otherwise the process can be repeated.

In an aspect of the image capture process described in FIG. 21A, a simplified drawing is shown in FIG. 21B. Here a camera and display combination 2150 (which may be the user glasses or some other camera/display device) captures the image of the fiducial marker 2154 and provides a display of the image on screen. The image of the fiducial marker 2152 has a match score 2156 associated with it. The image presented represents an enhanced reality camera image (I_{c}).

In some embodiments, there can be an Enhanced Reality Engine (FIG. 22A) to produce an enhanced reality image. In some embodiments, the system reads the marker depth data (Dₘ) 2204 and computes a depth of the virtual deformable model with respect to the marker depth (D_{md}) 2206. Image data can be continually fed to the system via a camera looking over the patient 2218. The computer can determine “vergence” corresponding to the model depth D_{md} 2208. “Vergence” may be thought of as the angle between the lines of sight for the left and right eyes to a target object being looked at, to accommodate a focus comfortably at a known depth. Thus, when the object being looked at is far away, the left and right eye lines of sight are parallel. If the object is close, then the left and right lines of sight can be sharply angled. In some embodiments, the D_{md} may be estimated from other cues in the user environment, including but not limited to the depth of the HCP's hands from her eyes, using the fact that a good hand-eye coordination would mean eyes will focus where the hands are working. In some embodiments, the depth of HCP's hands from her eyes can be estimated using unique gloves she will wear, that will have unique visual (infrared or visible light) features, active or passive, that are readily ‘seen’ by our system and processed. In other embodiments, other parameters (e.g. length and direction of gaze, knowledge of workspace location on the OR table, etc.) about the HCP may be sensed and used to refine the estimate of D_{md}. In some embodiments, the depth estimation is not to the hands but to the region where the medical procedure is taking place in the patient (the area of actual procedural concern). The system then reads model; M′_{I}, I′_{c}, T_{CT}, 2210 which are received from other processes and uses all of them to render a left and right enhanced reality image using the correct vergence information, focused at depth D_{md} 2212. The image data can then be sent to a display device 2214, which may be a wearable display.

In one non-limiting example, the user may wear glasses having a left panel 2230_{L} and a right panel 2230_{R} (FIG. 22B). The two panels can be a display device as described elsewhere herein, or a third-party display device suitable for use in this example. The display panel can display computer generated images and allow a user to see the real world at the same time. The glasses (shown here only as a representative scheme) may have a camera 2252. The process used to generate the enhanced reality image accommodates each individual user inter pupillary distance IPD and vergence V. This allows a user to “see” the scan image model 2250 at the proper depth, taking into account the read depth of the fiducial marker 2240 Dₘ, and the computer model depth D_{md} and the vergence for D_{md}.

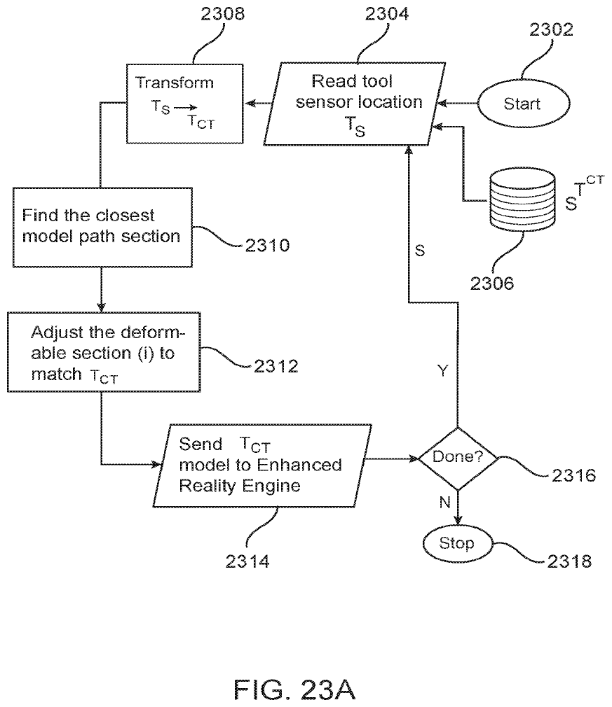

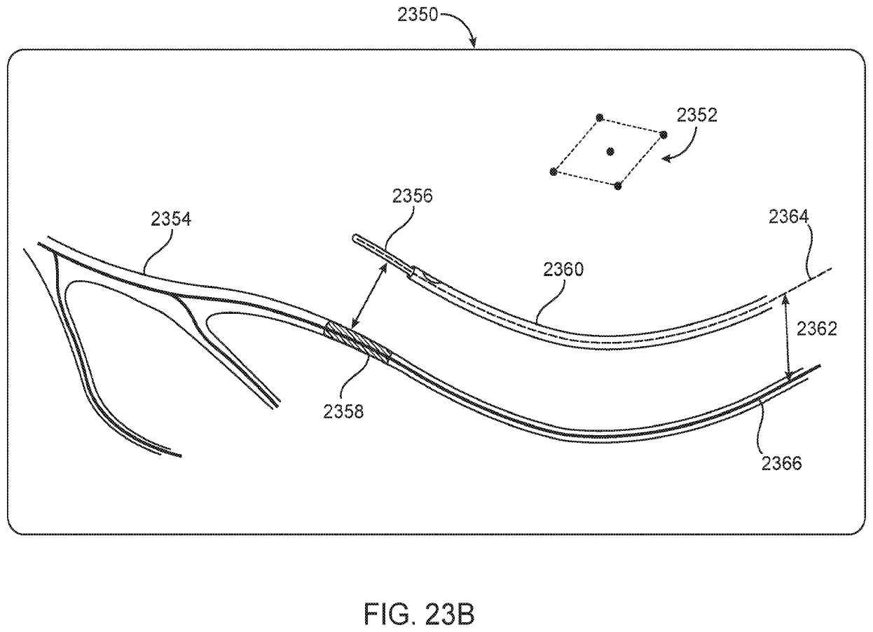

In another embodiment, there are methods for enhanced reality tool tracking (FIG. 23A). In an embodiment, the enhanced reality tool tracking begins 2302 when a user requests the image or the system starts in response to a predefined instruction. An electromagnetic sensor can track the position of various tools and SDD markers inside the patient body 2304. Additional data such as scan image data or other data may be received from the system or computer memory or other external source 2306. The system can perform a transform on the read tool sensor location with the image scan data and/or other data input 2308. The process finds the closest model path section 2310 and adjusts the deformable section (i) to match the newly transformed data T_{CT} 2312. The T_{CT} model is sent to the enhanced reality engine 2314. The system then determines if the process is done 2316. If the process is not done, additional transform data can be generated by returning to the read tool sensor step 2304. Otherwise the process can terminate 2318.

In a non-limiting example, the process of enhanced reality tool tracking can be thought of as pushing sensed objects into real positions with allowances for dramatic errors that cause the operation to fail, restart or alert the user to the issue. The visual example (FIG. 23B) shows an enhanced reality view 2350 having a blood vessel (or other feature) modeled as a deformable model wall 2354. The image for the deformable model wall is based on the scan image data with one or more marker reference patterns 2352. In addition to a deformable model wall 2354 the model also possesses a deformable model path 2366, also based on the scan image data. The deformable model path is the estimated path for a minimally invasive device to follow as it approaches or resides in the vessel for the medical procedure. The electromagnetic field sensor can detect the catheter, guidewire or any other tool having an appropriate SDD marker on it, and the system can use the electromagnetic sensor data to provide a sensed position for the SDD of the medical tool 2356. The tool may have SDD markers along its length allowing for the system to make a sensed tool representation 2360, and a sensed path 2364. The process can then transform the position of the sensed tool and path on to the image scan data path, putting the sensed tool 2356 into the closest path section 2358 of the anatomy model. The sensed positions of medical devices are shifted by a distance 2362 to the actual positions of the anatomy. By using various SDD markers in the fiducial marker and the various tools, the system, through this process and others, can accurately track the position of each medical device in a body. The use of the SDD markers may be incorporated into any existing interventional tool, implant or therapy device that enters the body or is used on the body surface. The SDD marker may be used on diagnostic, therapeutic or cosmetic devices or tools. In some non-limiting examples, SDD markers may be used in infrared sensing probes, ultrasound catheters, stents, guidewires, pacemakers, endoscopes or intubation tubes, just to name a few.

In some embodiments, the SDD may be exterior to the patient body while the energy source or emitter is on a tool placed inside the patient body.

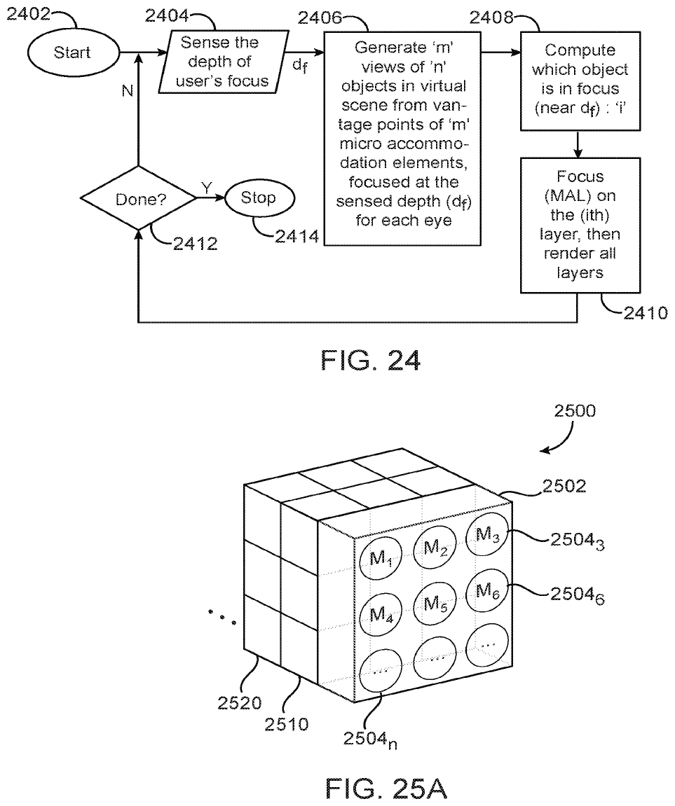

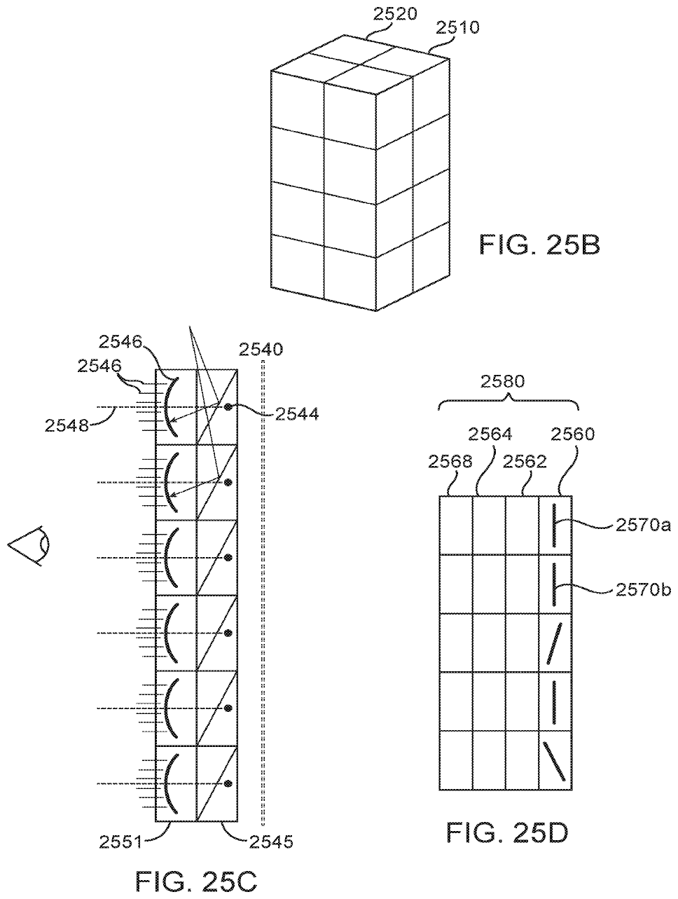

While there are various embodiments to the form factor and layout of the image system the user may wear, the image presenting optics are now described. In some embodiments, there can be a system and method for enhancing visual perception of reality using a micro accommodation layer (MAL) and translucent display stack (FIGS. 24, 25A-25D). In an embodiment, there can be a 3-layer stack with each layer divided into a like number of cells. In one aspect, there can be a 3×3×3 stack (FIG. 25A) having a voltage induced focus charging a micro accommodation layer 2502, shown here with ‘M₁₋ₙ’ elements 2504₁₋ₙ. The 3×3×3 stack is merely illustrative of a section of the combined display lens. The display lens for use in goggles, glasses or any eye piece, or display set up can be any dimension of cells. The middle layer may be a see The middle layer may be a see-through display with controllable fragments (n layers) 2510. The third layer can be a transparent support layer 2520 that may also serve as vision correction lenses for the user. In some embodiments, glasses or goggles can have two separate stacks, one used for each eye. The resolution of each micro accommodation layer may vary from 1×1 pixel per cell to HD resolution per cell. Data or video input can come from the system directly, or via a light engine.

In some embodiments, the see-through display layer 2520 and the lens array layer 2510 are juxtaposed such that the lens array elements allow focus onto the display layer using changeable focal length lenses.

In some embodiments, the wearable enhanced reality glasses can have two layers: a semitransparent micro mirror reflecting layer 2551, and a semitransparent display layer 2545. Light from an Enhanced Reality Light engine can enter 2545, reflect through the mirrors 2546 in 2551 away from the eye, to converge at distant virtual focal plane 2545 that is positioned at a comfortable accommodation distance from the wearer's eye. The mirrors 2546 may have their central axes 2548 parallel to each other as shown in FIG. 25C, or converging, focused on the virtual focal plane 2540, or diverging. The position of virtual focal plane can also be controlled programmatically by changing the focus and convergence of the micro mirrors 2546.

In another embodiment, there can be a composite enhanced reality visual computing chip 2580 (FIG. 25D). The computing chip may have a programmable lens array with tunable focus layer 2560 and a group of see through displays arranged in a single stack 2562, 2564, 2568. The visual computing chip may be used for RGB/HSV/Spatial and/or frequency domain filtering or display. The chip may be a programmable see-through display stack having a programmable lens array with tunable focus. During the procedure, the display chip or enhanced reality display may operate by sensing the depth of the user's focus (df) and then generating views of ‘n’ objects in one or more virtual scenes from the vantage point of ‘m’ micro accommodation elements, with at least some of those elements focused at the sensed depth.

In an embodiment, there can be a method for enhancing the visual perception of a user, using the micro accommodation layer and translucent display (FIG. 24). In an aspect, the method can sense the depth of the users focus 2404. The method can then generate ‘m’ views of ‘n’ objects in a virtual scene from the vantage points of the ‘m’ micro accommodation layer elements focused at the sensed depth (d_{f}) for each eye 2406. The method can then compute which object is in focus (near d_{f}): T 2408. The method then determines if it is done 2412 and either terminates 2414, or returns to the beginning.

In another embodiment, there can be a method to display an enhanced reality image to a user (FIG. 26). In an aspect of the embodiment, the method starts 2602 on a user command or automated command. An image can be captured 2604 (using wearable's camera). There are wearable position and orientation sensors (e.g. gyroscopes, magnetometer, electromagnetic sensors, etc.) 2606. The method then detects position and orientation of the markers 2608 using camera calibration 2620 and image 2604. The method then estimates the depth of an object 2610 from its pose (position and orientation). The method can render virtual objects with correct disparity 2612 and using camera calibration 2620. The method then displays the stereo image 2614 on to a left and right screen for a user's left and right eye respectively. If the process is done it terminates 2618, and if not done it begins again.

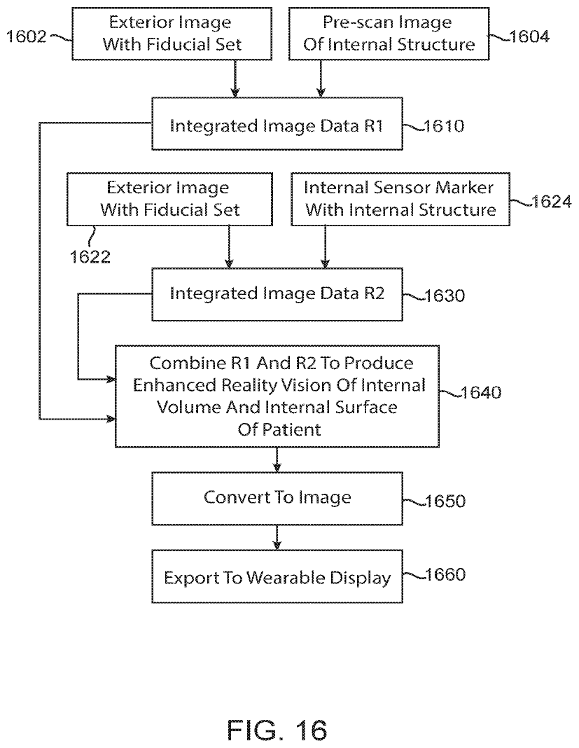

In an embodiment, the overall process for providing an enhanced reality surgical vision to a HCP involves collecting several types of image data, correlating them together, and presenting them as one image (FIG. 16). In an embodiment, the control unit can collect the exterior image of a patient having fiducial markers on the skin 1602. The control unit may also collect pre-scan image data on internal organ structure of the patient 1604. The system can then integrate the two images together to produce a first virtual 3D map R₁ of the patient volume in coordination with external fiducial markers 1610. The system may also use another exterior image set using fiducial markers having the same location as the first set 1622. The system then collects data from an internal sensor marker, such as a guidewire or catheter having sensor markers on them, and correlates it to the external image data using the fiducial markers. This produces a second set of virtual image data R₂. The two maps are then combined and correlated (R1+R2) to produce an enhanced reality vision of the internal anatomy of a patient (partial or whole anatomy) matched to the exterior fiducials 1640. The data can then be converted to an image 1650 and exported to a wearable display 1660. In some embodiments, the exterior fiducial image data may be the same data used to generate R1 and R2. This may be done when the fiducials remain in place for both interior scans of the patient. In some embodiments, the fiducial scans will be two separate scans, however the fiducials should be placed in as close to identical locations as possible for both scans to minimize the error when correlating the image data. In some embodiments, the goggles may also be tracked in the same 3D space as the patient and the fiducial markers on the patient. The position of the goggles can be measured relative to the other image data so the control unit can determine the proper perspective view for the image data when presenting it to the HCP. By doing a perspective analysis of the goggle position relative to the other image data, the HCP can see any aspect of the image data from the proper orientation of height, direction, angle and orientation to the patient.

In various embodiments the exported image to the wearable display 1660 may be a 3D enhanced reality image. In some aspects, the image may be a 4D image showing a time varying 3D map. In other aspects, the exported image may be any instantaneous 2D image, archived image, or any combination of image data the system has access to. In various embodiments, the export to the wearable display may be replaced with an export to any display device.

In various embodiments described herein, reference is made to various perspectives. Wearable's world refers to the view from the perspective of the goggles (the “wearable”). In some rare situations “wearable” refers to the outlook from a device worn or on the body of a patient, so context is relevant for the view point of a wearable. References made to the “world” of various image data sets refers to that particular image set being the “world” perspective viewed from. In some embodiments, reference is made to the wearable world, corresponding to the perspective of the wearable display device or the user wearing it. Tracking world refers to the perspective of the tracking of the fiducials on the patient skin. Interior world refers to the perspective of the organs within the patient body.

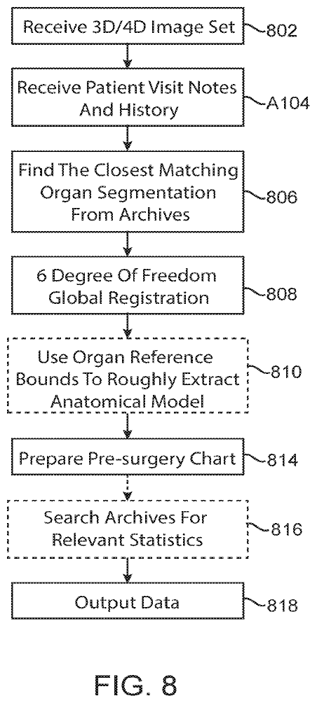

In various embodiments, there can be a process for capturing image information and data from one or more sources, and combining the image information and data to produce an enhanced reality image (FIG. 8). In an embodiment, a control unit may receive 3D/4D image data 802 (such as from a medical imaging system, or archived image data from a data repository). If the patient is prepped for surgery and has fiducials, the image data may include a body surface image that provides a map of the body and fiducials. The image data 802 may be held in memory of the control unit while any patient data is received 804. The patient data 804 may contain information about why the patient is in for a procedure, what organs the patient needs to have operated on and any other relevant information about the treatment the patient needs. The pre-scan image data 802 and patient data including patient visit notes and history 804 can be analyzed by the control unit and the control unit may find the closest matching organ segmentation from the combined data 806. The control unit can then determine six degrees of freedom using a global registration 808. The global registration may use the pre-scan image data 802 combined with a surface image scan of the patient body. The patient can wear a set of fiducial markers during the surface image scan. In an embodiment, there can be three or more fiducial markers arranged on the patient body to establish three-dimensional reference points. In an embodiment, the fiducials may be presented in a nonlinear arrangement that will assist the system in determining a plane or three-dimensional shape in relation to the body. In another embodiment, the fiducials may be positioned in predesignated places that can be correlated with relatively high accuracy to features present in the pre-scan image data. The system may use an organ reference chart to provide boundaries to roughly extract the position of the organs or anatomical model 810. This enhanced reality data may optionally be stored in the patient medical record. Once the pre-surgery chart 812 is prepared, the system may optionally search data archives for relevant statistics 814. The pre-surgery chart 812 can then be output 816 to any one or more of; data archive, control unit, computer display or wearable display. This process may be repeated as often as desired.

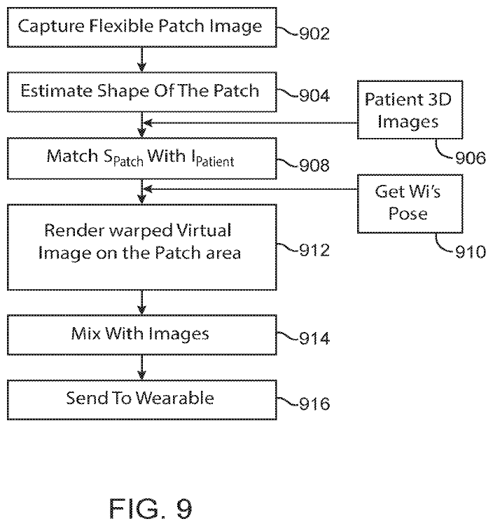

In various embodiments, the integration of pre-scan data types with patient medical records, and real time images can be presented to a health care provider (HCP) via a computer screen, or a wearable display unit (FIG. 9). The control unit can combine any combination of patient record data, pre-scan image data, enhanced reality imaging or any other content the control unit may be able to present and present that data to the wearable display. In some embodiments, the wearable display unit may use a transparent display screen such as OLED. This allows the HCP to have normal vision with the HCP's eyes seeing what is ahead of the HCP, as well as projected images from the control unit of computer generated images, such as data, enhanced reality images or the like. In an embodiment, the wearable display may have a camera able to sense fiducials on the patient body. The fiducials may be arranged around the surgical site like a patch or outline garment. The wearable display camera can capture the images of the fiducials 904 and transmit the data to the control unit, which can do the image processing required to combine the pre-scan image data 906 with the fiducial information 904 and any real-time sensor tracking images. The control unit may then adjust the data of video imagery with the position of the wearable camera 910, which may vary due to the position and orientation, height or angle of the HCP wearing the wearable display unit. The system may recognize the fiducials by shape or by some other feature readily distinguishable by the system and not confused with other fiducials. In an embodiment, there may be three fiducials having a visual distinctiveness for a HCP to discern (e.g. triangle, square and circle shapes), while optionally having a data pattern the control unit can recognize (e.g. barcode, UPC code, 2D code, etc. . . . ). The control unit can adjust for the point of view from the video camera 912. The control unit can then warp a virtual image of patient's internal anatomy to match the sensed shape from 904; and draw it right over the patch area in the patch image (902) from the wearables point of view. This can give the perception of ‘seeing through’ the patient's skin to the HCP Once the fiducial image data is ready, it can be combined with the pre-scan data to produce a pre-scan image combination (R₁) 914. The pre-scan image combination may be sent to the wearable display device 916. The image combination process may be performed any number of times, and include data smoothing or averaging to facilitate the combination of the two image data types.

In another embodiment, the HCP may wear glasses capable of rendering computer images on the goggles. The goggles may be VR or AR type glasses, or alternatively may be enhanced reality glasses (ERG) as described herein. The HCP may receive continuous updates from the control unit that allow the HCP to have a streaming image of properly rendered images with a minimum of error in the image overlap between scan image data and real time image data.

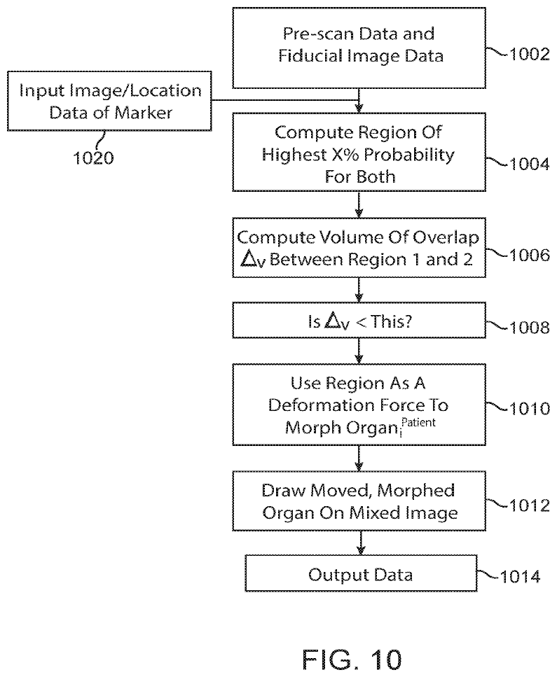

In another embodiment, image data may be augmented using live location data from an invasive probe (FIG. 10). In some embodiments, existing image data may be received from any source, and enhanced using an invasive probe. An invasive probe may be advanced into a patient along a generally known path. The probe may have one or more markers (which may be passive, active, or a combination of both) that can be detected by sensors of known location and position relative to the markers. The control unit can begin with the combined image data 1002 of the pre-scan image data (i.e. CT scan showing internal body organ of interest) and the fiducial data of the patient (fiducial markers on the exterior of the patient as described herein). A device having one or more sensor markers is then advanced into the patient body, and paused along the track of advancement at preselected distances. The sensor marker locations can be captured at these paused positions to produce an input image showing the location of the sensor markers relative to the fiducial markers on the patient body 1020. In an embodiment, the snap shot of the sensor markers inside the patient body may be taken at gated intervals matching the gated intervals of the pre-scan images. The image from the sensor markers and the combined image from the pre-scan and fiducial markers can now be combined. The control unit may then compute the region of highest probability 1004 for the position of any organs, blood vessels or other features in the patient body. The control unit compares the location data of the patient fiducials and internal organ image combination against the location information of the probe markers relative to the fiducial markers 1006. The two image types having in common the fiducial markers placed in the same location on the patient in each image combination. The control unit analyzes the two combined image data sets to compute the volume of overlap (Δᵥ) between the region of the tissue of interest of the pre-scan image combination (R₁) and the region of the probe marker image combination (R₂). If the volume of overlap (Δᵥ) is within an acceptable margin of error for a particular procedure 1008, then the volume of overlap can be accepted and the data from R₁ and R₂ may be combined. In combining R₁ and R₂, the pre-scan CT images may be altered in a pattern fitting program to make the pre-scan data morph into the most acceptable shape for the organs to match the organ data from the sensor marker scan 1010. The deformation method to morph the organ(s) may include but not be limited to data smoothing program, curve fitting program, a graphics processing program, or other process to help make the organs of the two combined scans fit into a single model. That new single model can then be converted to display data 1012. In some embodiments, the display data may be optimized for display on the wearable device for acceptable performance. In another embodiment, the pre-scan image data of the organs of interest can be morphed using a program that adapts the organs by the relative shift in the organs detected by the sensor marker scan. Various other embodiments may include three-dimensional image data averaging, data smoothing using various algorithms, and data smoothing based on user inputs. In some embodiments, any or all of the image and/or data processing operations may be cached as live operators with a raw combined enhanced reality data field set, and all the processing done on the fly. The final product of the image smoothing/organ morphing procedure is an updated enhanced reality image 1014. The new image 1014 can then be exported to a display, data base or wearable device. In a medical procedure, this process may be repeated numerous times to provide a HCP with real time enhanced reality images of the operation volume.

The process of providing a user with an enhanced reality image showing a combination of real scan image data with computer generated imagery can be a complex process. Various safety and quality checks can be used to help strengthen the safety and reliability of the system, some of which are described herein.

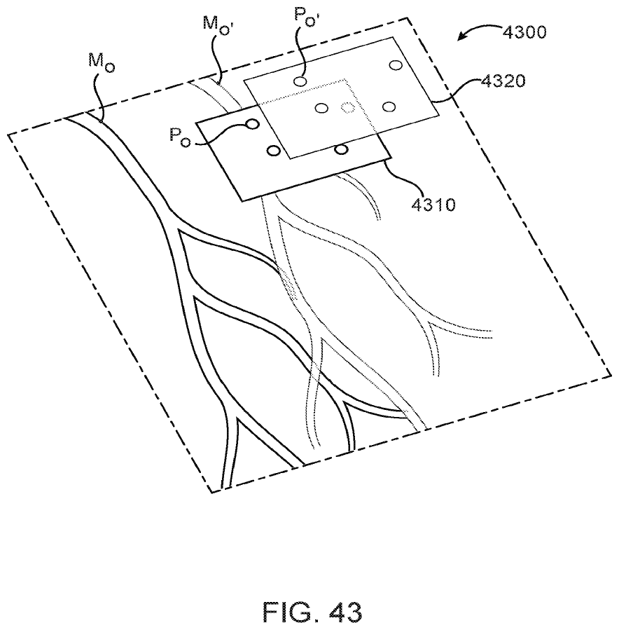

In some embodiments, a user check may present as the system showing the user an image with two views of the patient anatomy 4300 (FIG. 43). This case allows a user to correct for an error in the virtual environment. One view of the patient anatomy may be the scan image 4310 with the other image being the correlated image 4320 based on the detection of the fiducial markers and the calculated position of the anatomy of the patient (also the model image M). Thus in a “see through” set up, the virtual model M moves away from the Patient model with (ΔMo≈ΔPo). Looking at the figure, the change in the two view positions can be expressed as:

ΔPₒ=∥Pₒ′−Pₒ∥; and

ΔMₒ=∥Mₒ′−Mₒ∥,

where ΔPₒ is the displacement (shift) of a known Fiducial Marker between its real image co-ordinates (Pₒ), and sensed (live) co-ordinates (P′ₒ) derived from the electromagnetic/sensor system, and ΔMₒ is the displacement (shift) of a known point on the Model between its scan co-ordinates (Mₒ) and sensed (live) co-ordinates derived from the electromagnetic/sensor system.

To correct for the sensed shift errors, a second sensor can be used. The first sensor reading is with respect to the moved marker. Another marker can be used at a fixed location. This can ensure the relative distances of the two markers as they were in the original patient imaging scan. Using this method, the system can re-acquire the patient image scan position marker by using 3 dimensional cues.

One method to provide 3 dimensional cues may be to take advantage of a known marker position on the fiducial marker. A sensed tool may be used to make contact with a known marker position. The tip of the sensed tool can be identified by the sensory system so its position in space can be accurately tracked. The tool tip can then be placed into direct contact with the known marker position, so the sensing system can then accurately tell where the known marker is in actual space and time, based on the position of the sensed tool touching the known marker position. This provides a method the system can use to realign markers from the scan image position to the actual position.

This feature allows a user to do a quick visual sanity check and make sure the system is accurately overlaying the various images so the user can proceed with a medical procedure without being confused by the image integration.

In another embodiment, a safety check may involve determining if the sensed tool is in the intended work space, such as an organ, lumen or blood vessel of the patient body. In some cases the system may report the tip of the sensed tool is not within the designated workspace. When such a report is received, the system can verify the tool is in the proper work space using other detection methods. In this embodiment, the sensed tool may have additional sensors, such as a tactile gauge, a fluoro marker or a visual scope. Any of these, or a host of other methods/tools can be used to tell via a data read out, user observation or indirect imaging, if the tool is in the proper work space, of if the tool has somehow perforated the tissue and is outside the intended work space. Being able to verify the tool is in the proper space or alignment lets the user know of the problem, and confirms the system is not erroneously reporting the position of the tool.

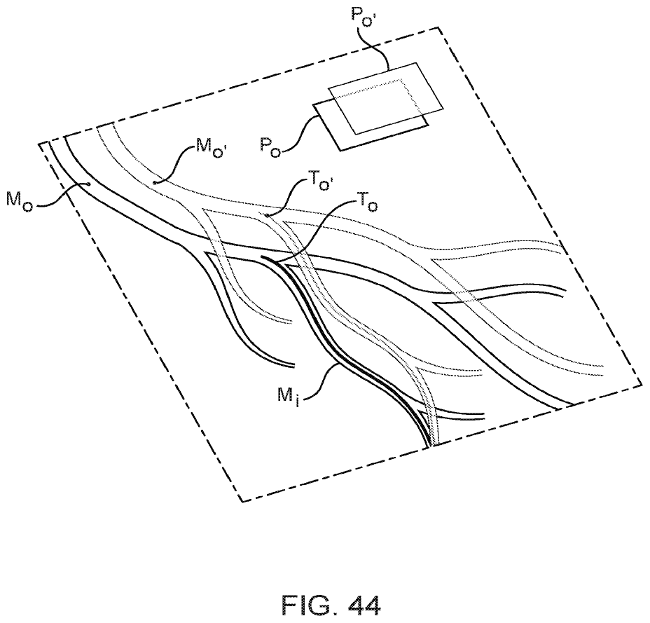

If the tool is in the proper work space and the tool position is not properly represented in the enhanced reality image, the system may engage another correction to restore the proper image presentation. In an embodiment, the patient marker fiducials are correctly aligned and verified (so Pₒ′ overlays on Pₒ in the enhanced reality image), but the reference (Mₒ) and sensed (Mₒ′) co-ordinates of a known model point do not line up and the system detects or predicts that the tool is physically constrained within the cavity or organ boundaries (FIG. 44). In some embodiments, the system may transform the closest workspace subsection Mᵢ to move over to align Tₒ with Tₒ′. Mo, Po, To are all objects in reference to the scan image (or the CT space). Mi is a subsection of model M, like a tree branch (i=0 makes it the root branch). Mo′, Po′, To′ are the same points, but after applying registration transforms. In theory they can align properly with their reference counterparts. But there may be non rigid motions so they won't align. The non rigid motion may be described in three categories:
(1) Due to the marker patch (Po) motion on skin;
(2) Due to model (Mi) movement, and
(3) Due to Tool perforating out of the model constraints.

For each of these sources, The system may use the following corrections:

(1) Use another sensed marker point (a sensed tool), touch Po with it, and use the offset in them as correction. (2) Assume or ensure that the tool is still constrained within the model, then trust the tool position (To′) to be the ground truth for where the Model should be as well. Then the system can move the relevant model section or branch (Mi) to encompass the sensed tool location (To′).

The various embodiments can now be viewed in a few examples where the technology described herein may be used.

### Example I: Patient Registration

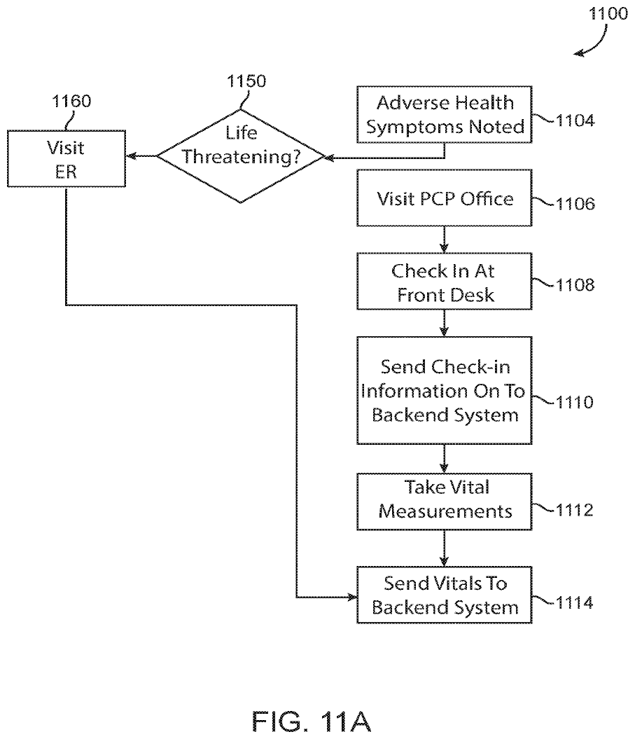

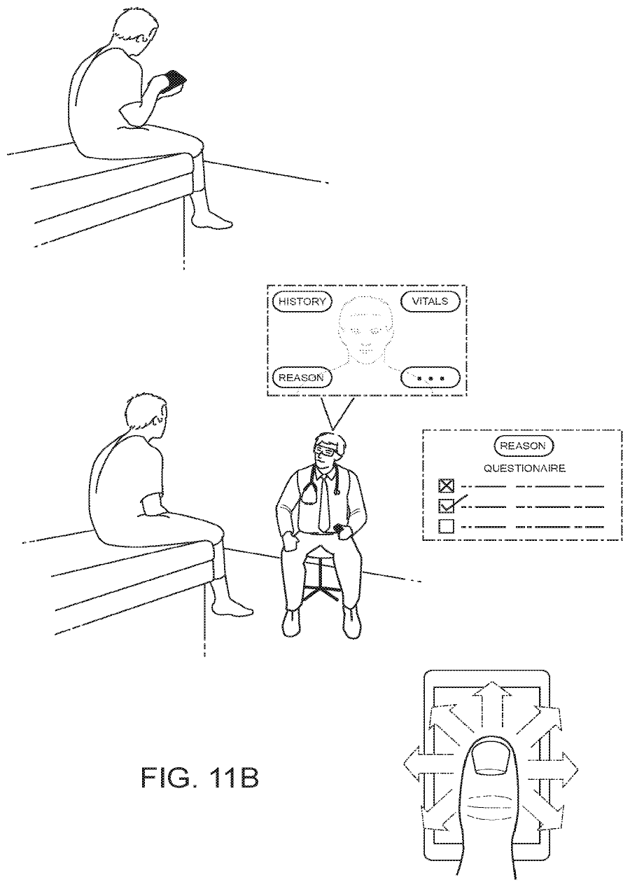

The devices described herein may begin to work with a patient for diagnosis and treatment planning the moment the patient enters the health care system. Many medical records are stored electronically, and government issued insurance and benefits often encourage this practice. Electronic records may be correlated by patient identification, whether that identification is an alphanumeric code, social security number, or simply a patient name or designation. The patient may initiate a medical procedure with a health care provider, and take initiate steps for patient check-in (FIG. 11A). The patient can start by interacting with the HCP by either calling to make an appointment, or registering for an appointment online 1102. During the initial interaction, the patient can be queried as to the reason why the patient is seeking medical help, and any adverse health symptoms can be noted 1104. If the patient's condition is urgent or life threatening, the system or the HCP can redirect the patient to visit the nearest emergency room 1160, or dial 9-1-1 for immediate assistance 1150. If the patient condition is not urgent or life threatening, the patient may proceed to visit the HCP office 1106. The patient may check in at the front desk, receptionist or other administrative point where the patient health insurance, records and other information can be correlated to the patient and verified 1108. Once the check-in information is completed, it can be sent electronically to the backend System” 1110. The patient vital measurements (height, weight, allergies, medications, etc.) may be taken 1112 and that added vital measurement information can be sent to the backend system 1114.

Wireless devices such as tablets, smart phones and laptop computers may be used to gather the administrative information, vital measurements and any other patient data desired. These wireless devices may be connected to the backend system through the cloud so any and all updates may be made continuously if desired. Alternatively, the data may be pushed to the backend system only at specific intervals (based on time, or on commands from the HCP). Data may be thought of as being sent incrementally at specific steps, data in actuality can move back and forth between the HCP and the backend system or control unit continuously.

The manner of initiation is not critical, so long as there is some way for the health care system to register the patient interest in medical treatment and/or diagnosis. Once the patient can be identified, the system may take note of any symptoms the patient describes. Notation may be by patient input into questionnaires (paper or electronic), verbal questions by a health care provider or ancillary service. The back-end system may be a computer on premise, or it may be a centralized data repository. The backend system may involve numerous computers and storage drives amorphously in the cloud. Data may be transmitted securely, and/or stored at secure facilities that ensure protection of patient data, while processing may be done in those same locations, or at various other computer locations.

The process of the example can be seen with the patient entering data in an examination room 1120 (FIG. 11B). The HCP may use the enhanced reality glasses while discussing the patient's concerns 1122, so the HCP can see the various medical records of the patient while holding a UID 1126 The HCP can scroll through questions or other information screens displayed on the glasses, and input information via the UID 1124.

### Example II: Patient Examination

In another example embodiment, a patient may be viewed by a health care provider and the health care provider may opt to engage the enhanced reality system in the event the patient is not already in the system. This may be done at any time the during or after a patient visit to see a health care provider, or any time during or after the patient engages in a consultation with a health care provider over the phone, via internet connection (video conference), chat (delayed text or voice communication over the cloud), or other methods of communication.

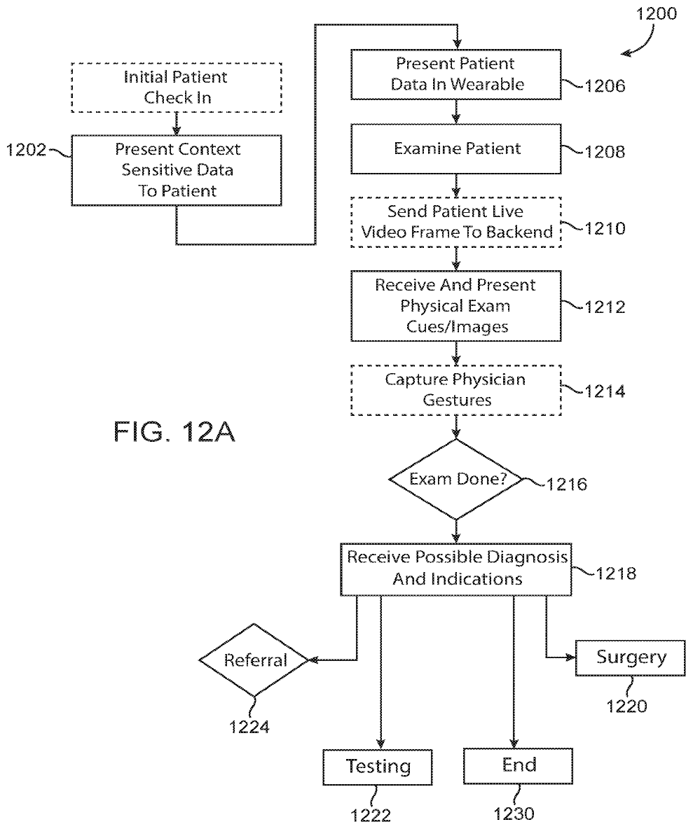

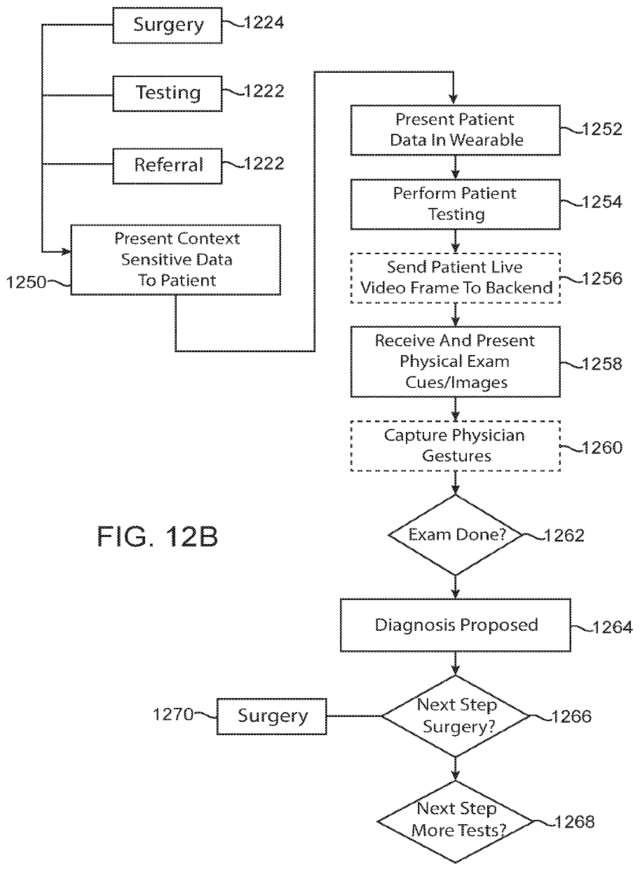

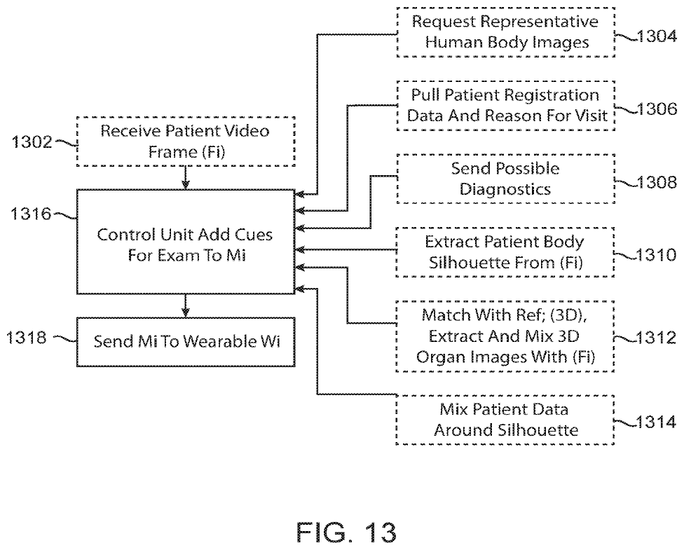

In this example, patient data may come from an initial check-in as described herein. Alternatively, patient data may be retrieved from storage when the HCP is in the examination room with the patient (FIG. 12A). The HCP may present context sensitive data to the patient 1202, and discuss the health condition and symptoms of the patient. Data from the backend system relevant to the patient condition may be displayed on a wearable display 1206. The HCP then proceeds to examine the patient 1208. If the patient agrees, video of the examination may be taken and send to the backend system 1210. The added data from the examination, including any video, can be analyzed by the backend system and provide updates into the wearable display of the HCP 1212. These updates may provide additional cues or queries for the patient as the backend system may need or request additional data to narrow the issues concerning the patient health. If the HCP engages in any gestures or semantic examination elements (i.e. striking a knee with a rubber hammer), that may also be recorded and sent to the backend system. When the examination is completed, the HCP can signal the system that a diagnosis should be issued 1216. The system can then produce a diagnosis and indications with suggested treatment 1218. At this point the HCP can conclude the patient examination with a diagnosis and solution 1230, recommend additional testing 1222, refer the patient to another HCP 1224, or refer the patient to surgery 1220.

### Example III: Pre-Procedure Examination

In another example embodiment, the patient may require additional screening to determine the cause of symptoms, or to treat an identified health condition. The patient may enter a pre-surgical examination from a referral, additional testing or simply show up for a scheduled surgical procedure (FIG. 12B). In this example, the HCP may again present the patient with context sensitive data and verify any information in the patient record so far 1250. The presentation of the data may be in a wearable display 1252. If the patient is in for additional testing, screening or referral, the HCP can conduct those services with the aid of the enhanced reality system and have data presented to the HCP through the wearable display 1254. If the patient consents, video of the additional procedures may be taken and sent to the backend 1256. The HCP can now use the system and the enhanced reality images to illustrate to the patient the nature of the medical condition to be treated, and how the treatment should work. The patient may visualize what the HCP proposes to do through a video monitor or a visual headset specifically for the patient to see. The system may present to the HCP and patient clarifying inquiries to further refine and detail the diagnosis so far 1258. If any gestures by the HCP are part of the additional examination or procedure, those gestures may also be recorded and sent to the backend 1260. The HCP may indicate when the examination is finished 1262 so the system may produce a proposed diagnosis and solution 1264. The HCP can make the determination and recommendation for the patient to proceed to surgery 1266. If the patient consents, and the patient is prepared, surgery may be conducted next 1270. If additional testing is indicated, the patient can be referred to additional testing 1268.

### Example IV: Surgical Procedure

In another example embodiment, a patient may undergo a surgical procedure with a HCP using the systems and methods described herein. The surgical procedure is not limited to one kind of surgery. The patient may undergo a minimally invasive surgery (MIS) or open procedure. In an example embodiment, the HCP may use a wearable display device connected to a control unit or backend server. The control unit can draw in data from various sources. The data sources may be image data from the wearable device camera, pre-scan image data, data from the patient records, data from recent patient examination, or data from public data sources (internet). The systems may draw data specifics and combine them according to its programming to produce an enhanced reality image for the HCP. In an embodiment, the control unit may receive patient video frame (Fi) 1302, request actual or representative human body images 1304, pull patient registration data along with reasons for surgical procedure 1306, send and receive possible diagnostic information 1308, extract the patient body silhouette from (Fi) 1310, match any of the image data with reference data, 3D data and extract and mix 3D organ images with (Fi) and mix the patient data around the silhouette 1314. Any or all of this information may be integrated into the enhanced reality image (Ei) 1316 and exported to the wearable display 1318.

### Example V: Generating Enhanced Reality Image with Insertion of a Sensor Probe

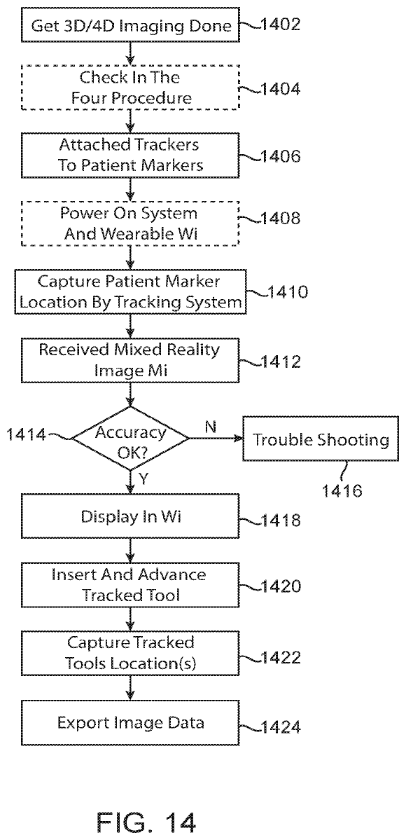

In another example embodiment of a surgical procedure, the patient may be prepared for surgery using an enhanced reality system (FIG. 14). The enhanced reality system may draw on any existing data 1402 prior to the commencement of a surgical procedure. The retrieved data can be archived in the control unit while the patient is prepared for surgery. While the patient is prepared, an optional check-in procedure may be done to perform registration data to the backend for validation and patient identification 1404. When the patient is set up for surgery, and before surgery begins, a set of fiducial markers may be placed on the patient body. The fiducial markers may be placed near where the entry point will be for the procedure (in the case of a MIS procedure), or the fiducials may be placed around the area of the body where the procedure is planned to take place (around the chest and heart area for a MIS aortic aneurism treatment). The HCP may activate the wearable display device 1408 and use the built-in camera to record the location of the fiducials, or capture the fiducials through some other tracking system that can feed the data to the control unit 1410. The system can then receive an enhanced reality image (Ei) 1412. The system may perform any number of safety and accuracy checks to ensure the system is operating within acceptable parameters 1414. If the system does not check out, the system can go through one or more trouble shooting steps 1416. If the system checks out ok, the image can be displayed on the wearable display device 1418. A tracking tool can now be inserted into the patient body and advanced into the realm of the fiducial markers 1420. As the tracking tool is advanced, the tool may be stopped periodically and detected by the appropriate sensor. The sensed position of the tracking tool can be fed to the system and the position data correlated with existing image data to refine the image of the body anatomy being treated in surgery 1422. In some embodiments, the tracked tool may have two or more markers on it so that when it is paused during advancement and tracked, the tracking unit can compare the movement and displacement of the most distal marker with the next distal marker, which in some embodiments may be now positioned where the distal marker was positioned at the first image capture time. By repeating the image capture as the tool is advanced, and having a separate marker at each location of previous detection, a higher level of confidence can be gained as body movement and range of displacement of the tracking elements are refined. All the tracking data can be used to enhance the image data. The updated image data is exported to the wearable display 1424.

### Example VI: Creating an Enhanced Reality Image Without a Sensor Probe

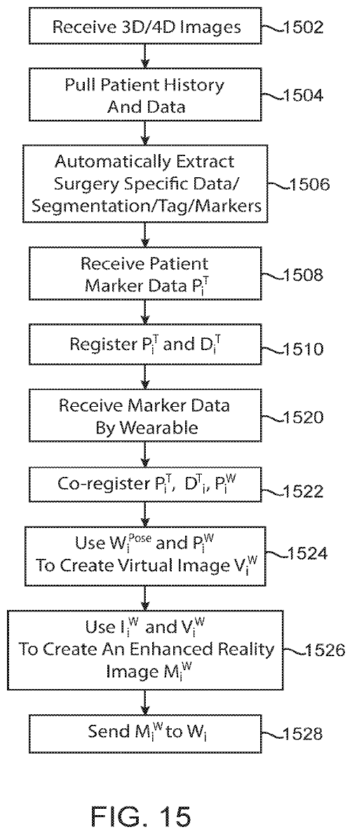

In another example embodiment, the control unit may receive 3D and 4D images from any data source 1502 (FIG. 15). The image data here can be correlated to surface fiducial data, but the image data is from the perspective of the inside of the patient, the “inside” of the patient world. The system may optionally pull patient history and patient data 1504. The system can then automatically extract surgery specific data, segmentation, tags and markers 1506. If not previously done, the system may now coordinate the fiducial markers with the internal tissue image data, and coordinate the two data sets into one data set. This coordination of the two data sets produces a static data set of the position of internal organs to external fiducials (Dᵢ^{T}) 1506. This view perspective may be called the “internal world.” The system next can receive patient marker data (Pᵢ^{T}). The patient marker data uses the same fiducial markers as those from the 3D/4D images 1502. In the initial gathering of the 3D/4D image data, the fiducial markers may have been passive, as any energy or active sensing of the fiducials may have interfered in the 3D/4D image data generation. In the marker data process, the fiducials may be activated or plugged in to an energy or signal source so the fiducials emit electromagnetic energy (or other acceptable signal). The positions of the fiducial markers are recorded creating an image from the perspective of the outside or “tracking world” 1508. Here the patient may move normally, and the tracking of the activated fiducials follows the movement and rhythm of the patient, both for voluntary and involuntary movement. Using the position of the fiducial markers as a common guide, the position of the internal organs referenced to the fiducial markers (Dᵢ^{T}) can be registered against the patient marker data (Pᵢ^{T}) 1510. Next the system can receive marker data from the wearable (Pᵢ^{W}) 1520. The wearable's position relative to the fiducial marker (or the origin) can now be taken. The wearable position can previously be registered from a known position relative to the origin or fiducial markers. There may be an “initialization” position or orientation for the wearable device. So, long as the wearable is accurately registered to the system, the position of the wearable device relative to the fiducial markers can be taken and used to generate the perspective of the fiducial markers from the wearable position (wearable world). The system can now co-register the image data from the three worlds, the inside world, the tracking world, and the wearable world 1522. The system can adapt the image by using the position and orientation of the wearable in global space (vᵢ^{POSE}) with the patient visual sensor marker data in wearable's world (Pᵢ^{W}) to create a virtual image (Vᵢ^{W}) 1524. Next the system can use the wearable image data set (Iᵢ^{W}) and the co-registered data of the three world views to create a mixed enhanced image corresponding to the wearers perspective (Mᵢ^{W}) 1526 and export that image to the wearable display device 1528. This process allows the system to produce an enhanced reality image without using a sensor probe inserted into the patient body.

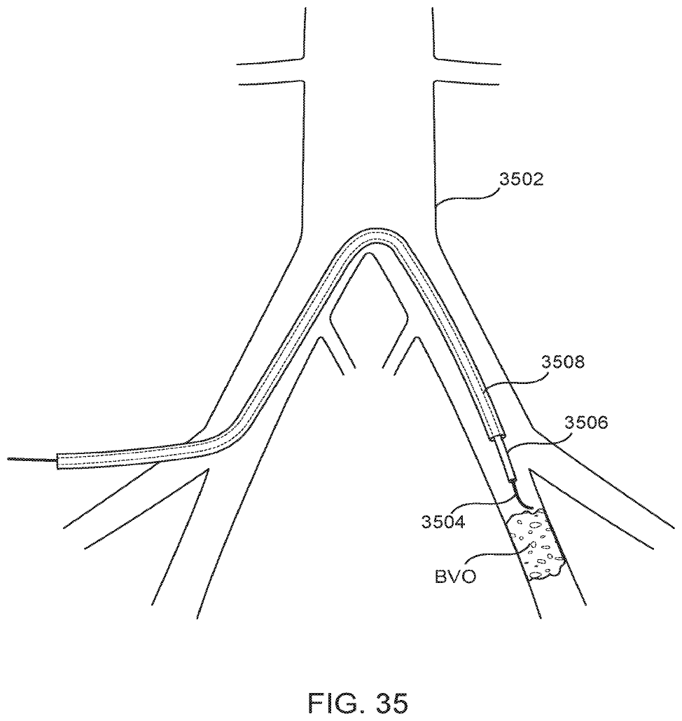

An example medical case is the need to treat a blood vessel clot or occlusion. Current methods involve entering a body lumen, such as a blood vessel 3502 with a minimally invasive device such as a guidewire 3506, guide catheter 3508 or generic medical catheter 3506 (FIG. 35). In this non-limiting example, a guidewire 3504 can be used to approach a blood vessel occlusion BVO. Once the guidewire 3504 is in place, a guide catheter 3506 can be advanced to the general area, and a medical catheter can be deployed within the guide catheter. The wire or catheter can be used by a HCP to try and clear the occlusion.

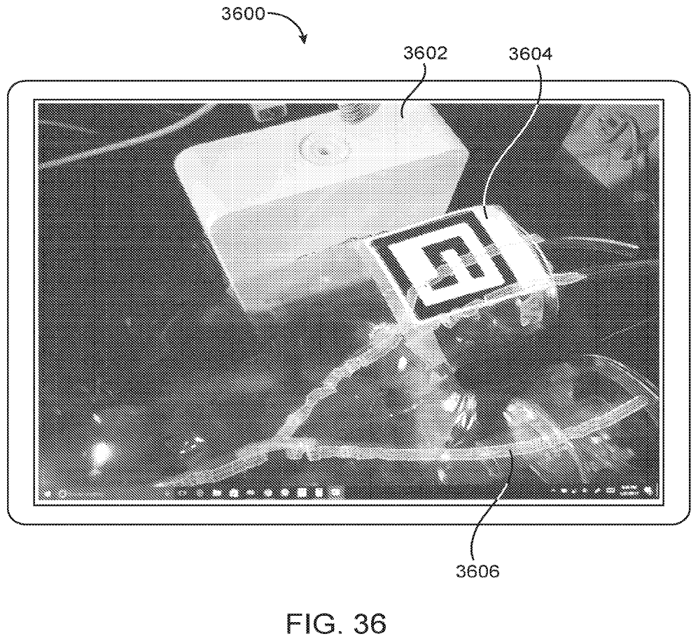

In one aspect of the systems, devices and methods described herein, there is a photo of a benchtop model of performing such a medical treatment (FIG. 36). The photo shows a model of a lower section of a human torso. A position sensing device 3602 sits close to the torso model. A fiducial marker 3604 has a visual print (visible) and a group of SDD markers (not visible). The camera that takes the picture can also be used as the camera to provide the visual image for the system and methods described herein to make the enhanced reality image shown. The enhanced reality blood vessels 3606 are projected into visual image such that they overlay on the model blood vessels inside the model torso. The user can see the virtual blood vessels properly placed in the image and corresponding to the positon of the model blood vessels in real time and on a continuous basis. A medical device having a SDD can be advanced through the model blood vessels, and its advancement is displayed in the virtual blood vessel and updated in real time. The demonstration model shows that the systems and methods do provide an enhanced reality image. If the surface of the torso were opaque, the virtual model would provide the user with a visible representation of the patient anatomy and procedural work environment in a three-dimensional view.

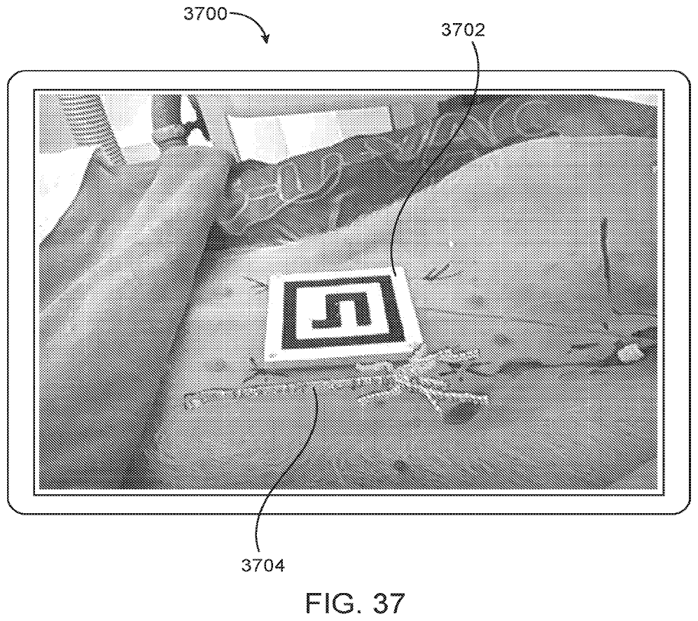

In another aspect of the systems, devices and methods described herein, there is a picture of a non-GLP, non-FDA study animal demonstrating the efficacy of such a medical treatment using the described technology (FIG. 37). A fiducial marker 3702 having a visual print and a set of SDD markers within it are used to help correlate the visual image with an internal anatomy image set and a sensed position field to generate the three-dimensional virtual model of the blood vessel 3704 where a doctor successfully placed a catheter into the animal, advanced it and manipulated the device based on the virtual image. CTA was used as a verification tool and did show the virtual model was accurate within the expected tolerances.

### Example VII—Additional Therapy Uses

A variety of specific embodiments and uses are described herein. It will be readily apparent to those skilled in the art that the enhanced reality imaging system described herein has a wide range of possible uses, both in and outside of the medical field. The enhanced reality system can be used for targeted energy delivery in cancer treatment, atherectomy or thrombus treatment. It may be used for localized drug delivery for cancer anywhere in or on the body. It may be useful for providing 3D viewing of tissue under any tissue being directly visualized, such as for cutting or ablative treatments, so a user knows how deep the tissue is to be cut. The visualization technology may also assist in any form of elective surgery as well. The ability to see past direct visualization may assist surgeons in determining the length and depth of any incision, and the possible risks to other tissues beyond direct visualization range. As such the enhanced reality visualization system may have benefits for those practicing robotic surgery or remote surgery.

In the realm of elective surgeries, the system may assist in fine definition of targeting adipose tissue for body sculpting procedures, directing energy or drugs to more precisely target tissue for treatment, placement of various implants with reduced risk of cutting nerve bundles or obstructing various body lumens.

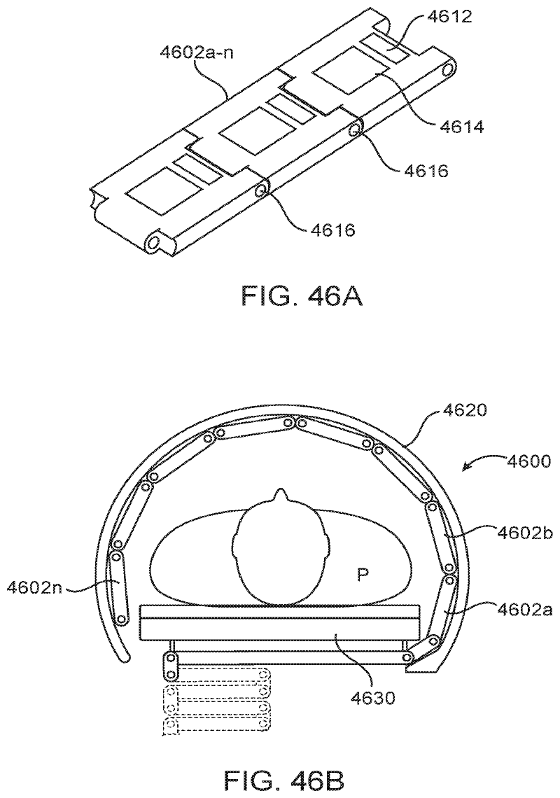

In another embodiment, the various aspects and embodiments may be adapted for stand off distance use with a patient who should not have physical contact with a device (i.e. a burn victim) (FIG. 46). In one aspect, the sensor garment may be modified to form a chamber, either a solid chamber or a chamber that can be assembled or constructed from readily available elements 4602ₐ₋ₙ. The patient P may lay on a table 4630 and have the chamber 4600 placed over them, or constructed around them. The chamber may be formed of pieces 4602ₐ₋ₙ assembled in a foldable manner, connected with a hinge 4616 piece. One or more pieces of the chamber 4600 may have a built in detector or micro x-ray source. Imaging sensors and fiducial markers may also be present (not shown) to coordinate the scan image of the patient, and the model image created using the various image scanners of the chamber. The chamber may also have a radiation shielding 4620 incorporated into the elements 4602ₐ₋ₙ, or a separate shield draped over the chamber.

### Example VIII—Post Procedure Uses

In post procedure situations, the enhanced reality imaging system may also be useful for reviewing a patient recovery. In an embodiment, a patient may be recovering from surgery in a hospital or at home, and be wearing a sensor garment properly aligned to the patient's anatomy. A user may do rounds or visit the patient (in person or virtually via a tele-health call), press a button and have the display device show a real time image of the patient area of interest, so a user can immediately see if the patient is properly recovering, or if the wound has some abnormality requiring additional treatment. The patient may also command for images to be taken at regular intervals and be sent to a remote user or physician for assessment. This interaction can also happen automatically and intelligently without the patient's initiation (remove or automatic image capture and transmission).

Additional aspects of the present invention are set forth in the following numbered clauses:
1. A method of producing a visual image data set from a visual image sensor containing at least one visual marker, the method comprising:
   identifying one or more visual marker(s) in at least one two-dimensional visual image;
   determining a depth and an orientation of the visual marker from the point of view of at least one visual sensor taking a visual image;
   establishing a three dimensional (3D) coordinate system for the visual marker(s) using at least one two-dimensional visual image; and
   creating a three-dimensional data set.
2. A method of producing visual image data set from a sensor image, the method comprising:
   establishing a three-dimensional coordinate system for a three-dimensional volume that is sensed by a position and an orientation sensor;
   sensing a position and/or an orientation of at least one of a sensor detectable device within the three-dimensional volume;
   assigning the sensor detectable device a volume, and an orientation in the three-dimensional volume; and
   creating one or more visual image data set indicating the position, orientation and volume of the sensor detectable device in the three-dimensional volume.
3. The method as described in clause 2, wherein the visual image data set forms a three-dimensional image on a display device.
4. A method of combining data types to create a three-dimensional image for a medical procedure, the method comprising:
   receiving at least one data set from a medical image scanner;
   receiving a least one data set from a position and orientation sensor;
   receiving at least one data set from a visual information sensor; and
   integrating the data sets from the medical image scanner, the position and orientation sensor, and the visual information sensor into a combined image.
5. The method as described in clause 4, further comprising exporting the image to a display device.
6. The method of clause 4, wherein the combined image is presented as a three-dimensional image appearing within the solid mass of a patient body.
7. The method of clause 4, wherein the display device is a three-dimensional display device.
8. The method of clause 7, wherein the three-dimensional display device has a left side and a right-side image display, the left and right side image displays being positioned at corrected focal depth and vergence for the wearer's individual eyes (left and right respectively).
9. The method of clause 4, wherein the position and orientation sensor is an electromagnetic field sensor.
10. A fiducial marker for use in a medical procedure, the fiducial marker comprising: a body;
a visually detectable feature visible on the surface of the body, the visually detectable feature having at least one visually distinct edge;
a plurality of sensor detectable devices, the sensor detectable devices positioned in the body;
   wherein at least one sensor detectable device is lined up with one visually distinct edge of the visually detectable feature.
11. The fiducial marker as described in clause 10, wherein the plurality of sensor detection devices are detectable by non-visual detectors such as X-ray imaging devices, electromagnetic sensors, diagnostic ultrasound equipment or other non-visible medical scanning devices.
12. A wearable display device comprising:
   a semi-transparent electronic display layer for receiving a combined image; and
   a structure support layer attached to the semi-transparent electronic display layer;
   wherein the structure support layer may provide vision correction to a user while the semi-transparent electronic display layer provides a computer-generated image of at least one internal detail of the object the user is looking at.
13. A flexible display for placement on a patient body, the flexible display comprising:
   a flexible body able to be draped onto a patient body, the flexible body having an upper surface and a lower surface;
a display screen incorporated into the upper surface; and
display electronics incorporated into the flexible body.
14. The flexible display as described in clause 13, wherein the flexible display has an aperture.
15. The flexible display as described in clause 13, wherein the flexible display has a stereoscopic three-dimensional image presentation screen or screen adapter.
16. The flexible display as described in clause 13, wherein the flexible display further comprises a position and orientation field sensor.
17. A wearable projection apparatus comprising:
a body having a body conforming contour;
a projector incorporated into the body, the projector able to project an image onto a surface; and
a position and orientation field sensor able to discriminate between an acceptable image display area and a non-image display area.

In an illustrative embodiment, any of the operations described herein can be implemented at least in part as computer-readable instructions stored on a computer-readable memory. Upon execution of the computer-readable instructions by a processor, the computer-readable instructions can cause a node to perform the operations. The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred, or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Further, unless otherwise noted, the use of the words “approximate,” “about,” “around,” “substantially,” etc., mean plus or minus ten percent.

Although the figures may show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A system for robotic assisted surgery of a patient, comprising:
a surgical or therapeutic tool;
an ultrasound imager;
an optical scanner;
a fiducial marker comprising a visually detectable feature configured to be detected by the optical scanner; and
a controller, the controller configured to produce two-dimensional or three-dimensional images of an area of the patient's body proximate to the surgical tool in real time based upon a pre-operative scan and real-time images from the ultrasound imager and the optical scanner.

2. The system of claim 1, wherein the surgical or therapeutic tool is configured to provide targeted energy delivery to the patient.

3. The system of claim 1, wherein the surgical or therapeutic tool is configured to be inserted into the patient's body.

4. The system of claim 1, further comprising a display configured to display the two-dimensional or three-dimensional images of the area within the patient's body in real time.

5. The system of claim 4, wherein the display is a holographic display.

6. The system of claim 4, wherein the display comprises glasses.

7. The system of claim 1, wherein the optical scanner is part of a tablet.

8. The system of claim 7, wherein the tablet further comprises a display configured to display the two-dimensional or three-dimensional images of the area within the patient's body in real time.

9. The system of claim 1, wherein the fiducial marker is configured to be in a fixed position relative to the patient's body.

10. The system of claim 1, wherein the controller is configured to correlate a position of the fiducial marker in the pre-operative scan with a position of the fiducial marker detected by the optical scanner to produce the two-dimensional or three-dimensional images.

11. The system of claim 1, wherein the controller is configured to determine a depth and orientation of the fiducial marker from a point of view of the controller to establish a two-dimensional or three-dimensional coordinate system.
